SEQUENCE LISTING (137 sequences)

SEQ ID NO: 1 - DNA, 2221 bp, H. Sapiens
  CDS (133)...(2055)
ccccctcctttttaagcgcctcccgccagcctctgctgtggctcgcttcgccgcgctccctccttccccgccttccatacctccccggctccgctcggttcctggccaccccgcagcccctgcccaggtgccatggccgcattgtaccgccctggcctgcggcttaactggcatgggctgagccccttgggctggccatcatgccgtagcatccagaccctgcgagtgcttagtggagatctgggccagcttcccactggcattcgagattttgtagagcacagtgcccgcctgtgccaaccagagggcatccacatctgtgatggaactgaggctgagaatactgccacactgaccctgctggagcagcagggcctcatccgaaagctccccaagtacaataactgctggctggcccgcacagaccccaaggatgtggcacgagtagagagcaagacggtgattgtaactccttctcagcgggacacggtacaactcccgcctggtggggcccgtgggcagctgggcaactggatgtccccagctgatttccagcgagctgtggatgagaggtttccaggctgcatgcagggccgcaccatgtatgtgcttccattcagcatgggtcctgtgggctccccgctgtcccgcatcggggtgcagctcactgactcagcctatgtggtggcaagcatgcgtattatgacccgactggggacacctgtgcttcaggccctgggagatggtgactttgtcaagtgtctgcactccgtgggccagcccctgacaggacaaggggagccagtgagccagtggccgtgcaacccagagaaaaccctgattggccacgtgcccgaccagcgggagatcatctccttcggcagcggctatggtggcaactccctgctgggcaagaagtgctttgccctacgcatcgcctctcggctggcccgggatgagggctggctggcagagcacatgctgatcctgggcatcaccagccctgcagggaagaagcgctatgtggcagccgccttccctagtgcctgtggcaagaccaacctggctatgatgcggcctgcactgccaggctggaaagtggagtgtgtgggggatgatattgcttggatgaggtttgacagtgaaggtcgactccgggccatcaaccctgagaacggcttctttggggttgcccctggtacctctgccaccaccaatcccaacgccatggctacaatccagagtaacactatttttaccaatgtggctgagaccagtgatggtggcgtgtactgggagggcattgaccagcctcttccacctggtgttactgtgacctcctggctgggcaaaccctggaaacctggtgacaaggagccctgtgcacatcccaactctcgattttgtgccccggctcgccagtgccccatcatggacccagcctgggaggccccagagggtgtccccattgacgccatcatctttggtggccgcagacccaaaggggtacccctggtatacgaggccttcaactggcgtcatggggtgtttgtgggcagcgccatgcgctctgagtccactgctgcagcagaacacaaagggaagatcatcatgcacgacccatttgccatgcggcccttttttggctacaacttcgggcactacctggaacactggctgagcatggaagggcgcaagggggcccagctgccccgtatcttccatgtcaactggttccggcgtgacgaggcagggcacttcctgtggccaggctttggggagaatgctcgggtgctagactggatctgccggcggttagagggggaggacagtgcccgagagacacccattgggctggtgccaaaggaaggagccttggatctcagcggcctcagagctatagacaccactcagctgttctccctccccaaggacttctgggaacaggaggttcgtgacattcggagctacctgacagagcaggtcaaccaggatctgcccaaagaggtgttggctgagcttgaggccctggagagacgtgtgcacaaaatgtgacctgaggccctagtctagcaagaggacatagcaccctcatctgggaatagggaaggcaccttgcagaaaatatgagcaatttgatattaactaacatcttcaatgtgccatagaccttcccacaaagactgtccaataataagagatgcttatctattttacacaa

SEQ ID NO: 2 - DNA, 16001 bp, H. Sapiens
ctgctgggaactaggagaaaaacaagactgaaatcatcactctagtgaggattacagagatataaaagtaactacagtatagtaaagtacaaaaatataggcatatacatgggatagaagcaacacagatgaaatagatgttcattctaagggacgaacagaaaaggcctgtgtgtgtgtgtgtgtgtgtgtgtgtgtgtgtgtgtgtgtgtgtgttagagacagagtctcactgtgttgcccaggctggagtgcagtggtgcgatctcggctcactgcaacctccgcctcccgggttcaagcgattctcctgcctcagcctcctgagtagctgggactaccggcgtgcgccaccacaccaagctaatttttgtatttttagtagagacggggtttcaccatgttggccaggatggtgtgtgtttaatttgagtgcagatgcccagaaaaagttcacagaagtgctctggaaacttcagagttcagtacgtaggagaaaaggaggcaagtggggatgtgggggtgacaacattagataaataggagtcagattatgaaggacataagatatcatgtgatggaaagagcctggaagagattttaagcttaagagtgatatgatctaatttttaatggtaaatcaaggggacagtataggatttatagggagagggggacttggtaattagagtcagaaagatatggattcagattccatttctcctgctaaccatttgtgtgactttgagcaaatgactcttttttttttcaatatacacaaagcaattttatttgtatgtacttgtggtaaacaattagaacatttcattcataataggataaaagtataaaatattcatgaataattttttttcagaacaggaatggaagtttattaaaaagctttagagcaggaatgaaagaaaggaaagtacacttggaagaggcccaagcaggcatcttggaggtcaagtgcggcatttgacctttgatttagggtgttatatgttggcatacttctggggtcctgcctccctttttccttgattcttcccttagggtgaccgagcaaatgactctttaagcccgtgtccacatatgtaaaatatagttgtgaaacagaatgagattatgcatgcagagttgctgacacagagcctggcacctactgtccatagcataaacattattcttctctcttaactttccaagtggatttgcatgcacagtgtggggagtggcttggtagggagaggggttggtgtttggagaggcaggagtccaagaagtgccttagaagctatcacctgaagccaggtgagagataagccctgaactaaggccctggccatggggatagttgcagacttgagggctaattgagaagtacaattccaaaggtcctggcatctgattagctatgcaggataagaatagtctagactgattccctttggggcaactggccagatgatggtgccatttgctgaggtaggaaaccatgagagcaggtgcaggattatggagaagatgatgaccttcaggtcaaccatgctgccaccagtgtgcacacctttaggcccaaaggttggccaagaggtatgtgtttgcagctgggtaagtgtggacagagtttggatctatgaactggggggaaagagatccaaatacatatacctgaggttgagtgtaggctagagcctgagggggaagagacggggaccagtagcaagcccgtggctggagaccagctctccttatcccactatgttccagcatagaactccaagaaatctgagaatcaagactgtttctgttgcaagtggcagaactccaattaaaattagcttgagcaagagaagaaatgcatcaattcctgtaactgaaatcaggaatccattgctcgccatctccctgctctgcttcctctaggctggcttctgtctcagccaggttcctgctttgcagtgccaagctaacactcagcagctccaggcttatattctaccagctcagcaactcagcagaaagagagctaacttccaaagttttagaaaaagtcccagagagggatctcatgcccatccttgaacatccttgattgctgtggccagagtgaggaacacaaaagttggctaagctgcaagcctacccccaagaggggtgacatcagacccacatgacaatacagactgaggagatggttccccaaaagaaaatcagggtgctattaccagaaaaggcgagtggatgctgagcagacacccatcatagatatgctgagtttgaggtgctatatagcattgtgggtgatatccagtggtctcaggttggagagagatgtcccaatggaggtatagcttgagaaatcacctgaggactttatgatggtagttgggtgggcagatgagtttcctcattggattgtggagagaaagaatgaccaggccgggcgtggtggctcacgcctgtaaattccagcactttgggaggccaaggcaggcggatcacgaggtcaggagttggagaccagcttggccagcatggagaaaccccgtctttacaaaaaatacaaaaaatcagccgagtgtggtggcaggcgcctgtaattccagctactctgcaggttgagacaggagaattgcttgaacccgggaggcggagtgtgcagtgagccgagatcgcgccactgcacgcactccagcctgggtgacagagtaagactccgtcaggggaagaggggatgtgccgggggaggagaatgaccagatcagaggaccaaggaagagacccaggggtagagagactccaccatttcatggcaaggcagaaaggagccttgatcaggaggagaaacggggatttaggagttggggactaagtggaccttagcattgggcacttggactacagaaaggcccaggactcagggtgtcgcgtccccaggaggaggaactggaaaggcaatgcccaaccccctaacttctagacgtctggcccctgtagtctcccgcccctggcctcgcccctcgttcctagcttgtttgccacctagtgtctctcccgggagcaagagtcctcaaagttacatcatgtgcggctgggagggcggtggcggatggggggcggggcctcgaagtcgggggccgaagagtggacccagtcctccaatgggagagatgggtttggcggtttggaggcaggggttggggcggcggctgggctgacctggagcctggagccccggggccgagggagctggcctgccagcggggcggaggaaagctagtgccagccctaccaggttccgcccccgcgcctgcccccctcctttttaagcgcctcccgccagcctctgctgtggctcgcttcgccgcgctccctccttccccgccttccatacctccccggctccgctcggttcctggccaccccgcagcccctgcccaggtgccatggccgcattgtaccgccctggcctgcggtgagtgacccccggcccggggcccacccgcaccttccgctgcgctcgccccctcggggctgccagtggcgctctcctgctctcagcctccgccaggtttcccatcctaggcggaggcgggcaggggcgactgctgtgggtccagcctcccgcgccgcgcgtctcttgggagggcagccggccggtgctcctcgtttccgcctgcacctccccttctctgcctcgctcgcctctgaccgcgcgatctctatctgccactctcagaacttcctctctctcctcgctcctctctgctgagccaggtctccgcatatcctcctttccttcccagatacctccctcggacctctaacgggctctcagccagcgccccagggtacttcgagaggcagcagggccctggggacaagggtacgtgagccccgggagactaagctcagagccccctaaagaaggtggaaggttaaatatccattcccggcctctcccggactggaaggactggaacctggcgggaagtccagagcagcccgagggacctgggcccaggggagggaggcaagcaaggtgggaggagggcgccaagttgccttcgtttcttacatagctggcttcttcctccgtccaggcctggagcccccaggctcgtcctgtttgtctgcctgtcctcttagtctcctatttattctctgaggcctctcttctcagcttttgtcccagagtcggaagtgacccacatctgtcgcacagcccgttccacttgggcagcccttgtgggtggtctctgaaggaaacgtcccacttagagggctgcaagagggtgtgggggcttcacaagagataacgtgagccaggctccagggagagagaggctgtcctcaagactgtgtgcttgaaaactgatgctcacggagaacttccctctgaggcaggaacagacccaggtcccagtagccctcctcccctgcccctggggccacactgatcatctatcctgctttagcggaaaccaccccagcttctaccccagacagactcaagctcccgtatccatgctctgagctttcttccttccccaggctaacaccctctgagtctgagctgccagcaagctgctgttccaccctcccaccaacaccaaagctctctaggcatgtggcctctaggaagaagagccaggggaagcacggggtcacgtggtcctgggtgtgggggcagtttctgatgggcgaggccttgatagaggaggagagtaacatccccttcatggtctttgctctctcgggtttactccaccttgagtccaggccaatcagagcagacgttgcttctctgtctcccagggccatgagaggacagacaacaggacgctgacctcctgagaattaagcccatgaaccccagccagtgacactcattccccagtggtcaaccttccgcagagttcagaaatacttacccgagggcaacattttatgcaaccattgttggtccaagtgggcagcagcagatcagggcctggaagcccagcatccagtcacctattctctgtgcaagagccctcatctagaaacctggcactggaaagactgtgacctttgcttggggctttcatagtcttacagcatacacaccagaaggaaagaataaacacagctgccattttaatttataaaaaactatacttgaaaatggaaataaaatggatgaggcttcaaataccagacatatgaaattgtcacctgggcccaacttcttgtcttgacacttgggccaaaggcccctactcatttcttttttttttttttttttttttttttttgagacagagtcttgctctgtcgcccaggttggagtgcagtgtcccgatctcggctcactgcaacctccacctcccgggttcaagcgattctcctgcctcagcctcctgagtagctgggactagaggcacacgccaccacatccggctaatttttatatttttagtagatggggtttcaccatgttgcccagtatggtcttgatctcctgacctcatgatccacctgcctcagcctcccaaagtgctgggattacaggcatgagccaccgtgcccagccatctccctacttatttctaaacgttgattaaacagttaaacatgggcatgggctacacaggcagtaacatcagcatgcctacatgtatacttccacacagccaggcatgtgccttttttgctcatttggccatatctgtccctcgctgagcaggagacaccctcctcaagcctcataaaggctacaagatacatgtgtcctgaacacatcccacacaccaactgcaacctgctcttcatggtccctgcatgcagacatgttttagcaggctgcagcccaagctttctgtctctccaccacctgccttgtccactctcgatgacagcaactagctcattgcctctgtttctcctataggcttaactggcatgggctgagccccttgggctggccatcatgccgtagcatccagaccctgcgagtgcttagtggagatctgggccagcttcccactggcattcgagattttgtagagcacagtgcccgcctgtgccaaccagagggcatccacatctgtgatggaactgaggctgagaatactgccacactgaccctgctggagcagcagggcctcatccgaaagctccccaagtacaataactggtaagccttgggctccacaacctgcaggataggtgcactgaggccactttgggttcaccaaggcaaaatcaacttaactagaacatcccaatggaatgaacaagaatgagagctttggggtaaacagacccagaaactgggatttgcttacgcctataatcccagcactttgagaggccaaggcgggtggatcaccaggtgtcgggagtttgggaccagcctgaccaacatggagaaaccccgtctctactgaaaaaaaaaaaaaaaaatacaaaaattagcccagcatggcagcgcatgcctgtaatcccagctacttgggagggtgaggcaggagaatcacttaaacccaagaggcggaggttgcagtgagccaagatcgcgtcattgcactccagcctgggcaataagagcgaaactctgtctcaaaaaaaaaaaagaaagaaactgggatttttttttttttttgagatggagtctcactgtatcacccaggctggagtgcagtggcatgatttcagctcacaacaacctctgcctccggggaattgtctcaagcaattctcctgcctcagcctccggagtagctgggattacaagcatgcgccaccacacccagctgatttttgtatttttagtagagacagggtttcaccatgttggccaggttggtctcaaactcctaacctcaagtgatccacccacctcagcctcccaaagtgctgggattacaggcatgagccactgcgcccagcctagtttgtagtttgtattttattttaatgttaaatgaagaagctgatataaataagatcctttgctttttttttttttcctcaccagttcagggagcttttgccaggggcagagacccccagagggctgggaccttggggaacaccccttagatgggacaaagcctggaggaagggactgagatgtgattgggtggggaaacataaggccaacagaagacctggagtcaaagttggacttgaaaaagtgggtctagggacaagggaaacctgctggccaccatcttcctgacaatcccctctcccccagctggctggcccgcacagaccccaaggatgtggcacgagtagagagcaagacggtgattgtaactccttctcagcgggacacggtacaactcccgcctggtggggcccgtgggcagctgggcaactggatgtccccagctgatttccagcgagctgtggatgagaggtttccaggctgcatgcagggtaaccagggcaggggcacagtggcaagggcacggaagatgtgaacaggtttggaacccttcatccaggggatgccttcctccacaggccgcaccatgtatgtgcttccattcagcatgggtcctgtgggctccccgctgtcccgcatcggggtgcagctcactgactcagcctatgtggtggcaagcatgcgtattatgacccgactggggacacctgtgcttcaggccctgggagatggtgactttgtcaagtgtctgcactccgtgggccagcccctgacaggacaaggtaagcacctgctctgccccaaggggaacacagaggccttcttgtactcagaggaaatcccaaatcctacctctccacagaccctaagaacctgtcctctctggcaacctaattcccaagatccagagcagcagtcccagcagagggataaggctgtgtttgcagagcactttgcactaggttgagaaaaatccgtgtccaagaataggggcatggaagctgatggttattatgaggtggggggcttcagccacctcttggtgctgctactgctcccaagtgtctctcctgccaatccctgatccctctggccccgacaccccagttcctgatgctgctgccagcagccccatgaccccattgtccccaggggagccagtgagccagtggccgtgcaacccagagaaaaccctgattggccacgtgcccgaccagcgggagatcatctccttcggcagcggctatggtggcaactccctgctgggcaagaagtgctttgccctacgcatcgcctctcggctggcccgggatgagggctggctggcagagcacatgctggtgagggcctggtgagaagcagggcagctgccggggacagggcaggggtggggcctggccagtctgcctcagcctcacctccctcctgccaggtgccaggctggtgggcggggactctacttgaaggcccaaagctttggcctcaggctgctgaatgttgaggtttcccctgccactaacccaggcctgatggcagggcaatcacttatatagttaataaacattggtcctccctattagaccctagctgcccttccccatgcagaccatgccctgacttttggtgacctctttcttattccctctctccccaatgcacagatcctgggcatcaccagccctgcagggaagaagcgctatgtggcagccgccttccctagtgcctgtggcaagaccaacctggctatgatgcggcctgcactgccaggctggaaagtggagtgtgtgggggatgatattgcttggatgaggtttgacagtgaaggtgagggactctcagatcatactcttggttctggctcttgtcagagcctcggggtctcctctctagtgttcacaatgactttgtcagtgagaaagtttcctgaacacccaaccctgctccattcctctggcagcccagccacccgagagacagcctttcctcatcagatcttgggtccatctcaggacaggggtgggtggagcaggaccttctttggtcttacatctcaagttttccttgtttggtccttcctttctttcacttctcctaacaggtcgactccgggccatcaaccctgagaacggcttctttggggttgcccctggtacctctgccaccaccaatcccaacgccatggctacaatccagagtaacactatttttaccaatgtggctgagaccagtgatggtggcgtgtactgggagggcattgaccagcctcttccacctggtgttactgtgacctcctggctgggcaaaccctggaaacctggtatgtgcggtggggaaggtgtggcacagcctccaggcctcagcaccttaatggtggaaaagctttctccacaacctccaaccatcttctaggactgccaggaggcacagaagtcatgaacgtttgcagtttccagtcccaggcaaaatctcagttcatgtcccaactccaccagtcactggttttgtgatctggctaagttgctcaacttccctaagctttagtttccacatcagttgaatgagggtagttgtgatagtacctatctcatgagattgttggaggattaaatagtgcataaaaagggtttatcacactgacaaatacacagtaaattctcaataataaatacaggctggatttttttttaatgaaaggaaaaggaaggacttttgaacattcttacagaaggtattgggctccaagcactatccataaagtttggcccattaggaaaagaggaaagctgcctcctctgctccaactctcctcctgccacttggctcccactgtcccctgtataataaccactgtctaaaggtcagtattgttaccgtcacccttcccctgtccctccaaagcattcaccccaatccttcctacaaacaaaatcaggtcagtgcttgagtctttcccagaagctagtttctgaatcctgtcattaccctgggcgcctgggagtcccacctctccctcagccctgcactctggaccttcagtattctttccatggccttctgcagtcaggcagtccagacaccaagaggcaggggcaaagaagagcatgggaggggaggctggccttgtagtcactgaagcctatattcaggtttgccaggctggcctagcagtcaccctccttgcttcatctaatcaccctttatttttactaacaccatcattaagcccccctcagccttcccacccaactgagaaatccaagaaactttcatctttccccacaggctagttccccaaccctttcatcatctccagatttgggggcataactagggcatcttgtccccagcttcaattcccagaataataccctgtgttaggattctgcactgggtgctgaagaaggatggctcttatctgcaatggcgggcagaagctggcggatgggagagggtggggattttggccccgtggcttccccactccccaggtctgaccagcaacctccagcagagaaggcaccatgtccactcaggggccacacagtggtgcttcatacatgtgccactgacttagtcccaacccccctccaggacacctgaaggtgccaagtgtgacctgggctcctgaggttatccctacccatgtgatatccctatctctatttttccagccctatcacttcatcagggtctaagcagggcagggaaatcaccaacatgttgttagctttaaaatcaattccttgcagggcacagtgactcacatctgtaatcccagcactttgggaggccgaggcagttggatcacctgaggtcaggagttcgagaccagcctggcaacatggcaaaaccccgtctccaataaaatacaaaaattagccaggcatggtggctcatgcctgtaatcccagctactcaggaggcaggaaaattgcttgatcccaggaggcagaggttgcagtgagacaagatcatgccactgcactccagcctggtgacagagtgagactccgtctcataactaaattaattaagtaaataaaatcaggccaggcgcagtggctcatgcctgtaatcctactactttgggaggccaaggtgggcagatcagttaaggtcaggagtttgaaaccagcctggccaacatggtgaaaccccatctctattaaaaatacaaaaaaatcagccaggcatggtggtgggtgcctgtaatcccagctactggggaggctgaggtaggagaattgtttgaacctgggaggcggaggttgcagtaagccaagattgcaccactgcactacagcctgggcaacagagcaagactctgcctcaaaaataaaaagataaaataaattcctatttgcatttggataacttaggagaacctgtcttccccggtttgctgacggaaagtcaattgtctgaagtactaagctgacattctcagtttttgctttaggtttgggtattcatttaaataataatctcacaaataatgaaatagtttctgggggaaaaattattataaccttatgcccatatctaaccccattcccttgagccctggtcagtgccaagtgccagtagcttggcacaaacattagtgccctgccaaaccccaattcctctcccactcttttctcacatagctcagctggccgcaccttcatggctaaacaacctgagctcttggagatgccctggctcccctctctctgctccttatcacacaaggttctaggcagctgatgaggcaaaaaaaaaaaaagaaccctgcaagaatgtgtgcccatgtatgtgtgtgttgggggtcgacatgaccttggaaataatagtgtttgtatttcctctgccaggtgacaaggagccctgtgcacatcccaactctcgattttgtgccccggctcgccagtgccccatcatggacccagcctgggaggccccagagggtgtccccattgacgccatcatctttggtggccgcagacccaaaggtaaacaacatatgagctccatgttcttggcaaaagggctatctctgtattagggcctacctccctccctctgatccagagcctcagcctggatctcacctttctccagagttctcccctggtgaatgcaaacttgggaggaggcaaagggtctgaaaatgggatagccgaggtcttaggagagagagtaccagtcaagctcaccagaagggctggagttagggtccaaagaaaagggctgcctgtgactctgttcattggtgatctaggggtacccctggtatacgaggccttcaactggcgtcatggggtgtttgtgggcagcgccatgcgctctgagtccactgctgcagcagaacacaaaggtgagcaccctcaccattcctccctctcctgtgtgtgcacacagcacgtcctctctcccttcctgagccagaccttccttttgtccacccctggagtctgatatggccccacctcttcccacttctatcttttccccatccctgaagatattcagaaccataagcctttcacagcttcctccaactggatgcagggtgcccttccctaccccagtgagaaggaagattccttacccatcttgcttcccccccagggaagatcatcatgcacgacccatttgccatgcggcccttttttggctacaacttcgggcactacctggaacactggctgagcatggaagggcgcaagggggcccagctgccccgtatcttccatgtcaactggttccggcgtgacgaggcagggcacttcctgtggccaggctttggggagaatgctcgggtgctagactggatctgccggcggttagagggggaggacagtgcccgagagacacccattgggctggtgccaaaggaaggagccttggatctcagcggcctcagagctatagacaccactcagctgttctccctccccaaggacttctgggaacaggaggttcgtgacattcggagctacctgacagagcaggtcaaccaggatctgcccaaagaggtgttggctgagcttgaggccctggagagacgtgtgcacaaaatgtgacctgaggccctagtctagcaagaggacatagcaccctcatctgggaatagggaaggcaccttgcagaaaatatgagcaatttgatattaactaacatcttcaatgtgccatagaccttcccacaaagactgtccaataataagagatgcttatctattttacacaagatttgtgctgttttcatttcccacctatcttcacaggcttccctctaacacctgtctcacaatcatcttcttccagcccctagaagaagcacagcctggcacaatcaaagatctgttttacaggtagctctagcactgggtcacagacataggaattgctgggagaaggcactatccactctatgtcctgagttcttaaaaaaaaaaaaatggtgaggctgggtgtggtggttcacgcctgtaatcccagcactttgggaggctgaggcgcacagatcacgaggtcaggggattgagaccatccgggctaacacggtgaaaccctatcgctactaaaaatacaaaaaaaaaaaaaaaattaaccgggagtggtggcgggcgcctgtagtcctagctatttgggaagctgaggcaggagaatggtgtgaacccaggaggcagaggttgcagtaaaccaaggtcgtgccactgcacactccagtctgggcaacagagcgaaactccgtcacaaaaaaaaaaaacaaaacaaaacaaaacaaaaaaaaaactgagggcctcagcaagctgctcagtacagcccccaagcctaaaattcctgatctcccacttagattgcagaagcctctacaactccattctccagtgaagtggcttcattgtcagttctcgaatttgttcttccccctgcctgacctggcactgggagctgcatagtattcatggaagcatattcaatattaggacagctaacaacacttctgtggcaccttctttatgccaggcactgctgagaccagctctgtcaaggagaccctaacccagcagtgctagaggaattaaaaacacgcacacagaaatatagaggtgtggagtgggaaatcaggggtctcacagccttcagagctgacagcctcgaacagagatttacccacgtgtttattgacagcaagtcagtgataagcattgtttttatagattaactaaaagtattccttacgggaaacaaagggatgggccaaaatgaagagatgggctctggctggttatctgcagcaggagcatgtccttaaggcacagatcgctcatgctattgtttatggtttaagaatacctttaagcggttttctgcccagggtgggccatgtgttccttgccctcattccggtgaacccacaaccttccagtgtgggtgtcatggccatcacaaacatgtcacagtgctgcagagattttgcttatggccagttttggggccagtttatggccagattttgggggcctattcccaacaaggcagtgttctaagcacatacctaacaaccctttgggggaattaccattttacagatgaagtaacaaaggcacagagaggtcaagtaatttgtccaaagcttcacagttagtaaacaatagagctaagggttaaagtgataaaactgcaaagacatgtctttcatagtagtatagacatctacaactgcaaggaccttagaagtcacctattccaccgggcgcagtggctcatgcctataatcccagcactttgggaggccaaggagggcggatcacctgaggttgggagttcaagaccagcgtggccaacacggtgaaaccccttctctacaaaaatacaaaaattagctgggcatgatggcaggtgcctgtaatcccagctatttaggacaatcacttgaactcaggaggcagaggttgcagtaagcctagatcatgccattgcactccagcctgggggacagagcaagactctgtctcaaaaaaataaaaaaataaaaagtcacctattccgtgtttctcaaacttaaaagtgcctttgaatcacctggagatcttgttaaaatgcagattgtcactcagcaagtctgtagtagcctcaagattttgttttccttacaagctcctgggtaatgctgatgctgccggtctgcgaactacagtttagagtagcctaaattcatctgaaataaactttggagagtaatcaggaaaacagtgcaatgatctcacatgagtcaggggcatcagcttaggataacttctgtgcaatctggatcaggctgagagaatgtataacctaaggtgggtagtgtggtagtctttacccagaaaaagggaacaaacttcaacaaataaaatggaattattgggaactccctcaaaggaaaaaactagaagaggaaacaccaagtgttctcataggaaatgccagtgaccagccagaatgtgtaaaaatctaaaggtcagagtggaacagaggttagaacaaagttagcagtggagtatattgtactttaaaacacagccagccaggcgcggtggctcacactttgggaggccgaggtgggcagatcacctgaggtcagcagtttgagaccagctagaccaacatggtgaaaccccatctctactaaaaatacaaaaattagccaggcatggtggcccatgcctgtaattccagctactcaggaagctgaggcgggagaatcacttgaaactgggaggctgaggttgcagtgagctgagatcacaccactgcactccagcctaggcgacagagtgagactctgtctcaaaaaaaaaataaataaataaataaatttaaaaatttaaaaataataataaaacacagccaaatccttagcttcataaatagcatatgataactgaataccgtcaaaagcaacatagcacaggtattaaacacatagtagacttgagccagaggtgcctctcagttgcttcatctgtaaaatggaaatgcctacactttacagtcttgttgggaagattaaaagagtcagt

SEQ ID NO: 3 - DNA, 2165 bp, H. Sapiens
  CDS (67)...(1989)
cccgccttccatacctccccggctccgctcggttcctggccaccccgcagcccctgcccaggtgccatggccgcattgtaccgccctggcctgcggcttaactggcatgggctgagccccttgggctggccatcatgccgtagcatccagaccctgcgagtgcttagtggagatctgggccagcttcccactggcattcgagattttgtagagcacagtgcccgcctgtgccaaccagagggcatccacatctgtgatggaactgaggctgagaatactgccacactgaccctgctggagcagcagggcctcatccgaaagctccccaagtacaataactgctggctggcccgcacagaccccaaggatgtggcacgagtagagagcaagacggtgattgtaactccttctcagcgggacacggtaccactcccgcctggtggggcctgtgggcagctgggcaactggatgtccccagctgatttccagcgagctgtggatgagaggtttccaggctgcatgcagggccgcaccatgtatgtgcttccattcagcatgggtcctgtgggctccccgctgtcccgcatcggggtgcagctcactgactcagcctatgtggtggcaagcatgcgtattatgacccgactggggacacctgtgcttcaggccctgggagatggtgactttgtcaagtgtctgcactccgtgggccagcccctgacaggacaaggggagccagtgagccagtggccgtgcaacccagagaaaaccctgattggccacgtgcccgaccagcgggagatcatctccttcggcagcggctatggtggcaactccctgctgggcaagaagtgctttgccctacgcatcgcctctcggctggcccgggatgagggctggctggcagagcacatgctgatcctgggcatcaccagccctgcagggaagaaggcgctatgtgcagccgccttccctagtgcctgtggcaagaccaacctggctatgatgcggcctgcactgccaggctggaaagtggagtgtgtgggggatgatattgcttggatgaggtttgacagtgaaggtcgactccgggccatcaaccctgagaacggcttctttggggttgcccctggtacctctgccaccaccaatcccaacgccatggctacaatccagagtaacactatttttaccaatgtggctgagaccagtgatggtggcgtgtactgggagggcattgaccagcctcttccacctggtgttactgtgacctcctggctgggcaaaccctggaaacctggtgacaaggagccctgtgcacatcccaactctcgattttgtgccccggctcgccagtgccccatcatggacccagcctgggaggccccagagggtgtccccattgacgccatcatctttggtggccgcagacccaaaggggtacccctggtatacgaggccttcaactggcgtcatggggtgtttgtgggcagagccatgcgctctgagtccactgctgcagcagaacacaaagggaagatcatcatgcacgacccatttgccatgcggcccttttttggctacaacttcgggcactacctggaacactggctgagcatggaagggcgcaagggggcccagctgccccgtatcttccatgtcaactggttccggcgtgacgaggcagggcacttcctgtggccaggctttggggagaatgctcgggtgctagactggatctgccggcggttagagggggaggacagtgcccgagagacacccattgggctggtgccaaaggaaggagccttggatctcagcggcctcagagctatagacaccactcagctgttctccctccccaaggacttctgggaacaggaggttcgtgacattcggagctacctgacagagcaggtcaaccaggatctgcccaaagaggtgttggctgagcttgaggccctggagagacgtgtgcacaaaatgtgacctgaggcctagtctagcaagaggacatagcaccctcatctgggaatagggaaggcaccttgcagaaaatatgagcaattgatattaactaacatcttcaatgtgccatagaccttcccacaaagactgtccaataataagagatgcttatctattttaaaaaaaaaaaaaaaaaa

SEQ ID NO: 4 - DNA, 3234 bp, Rattus norvegicus
  CDS (1)...(2268)
atgggacacggaatcacaaacctggaggccaacgaggagagcagagtgggctcagggttagaactggcgcttagacatatccaaggagttgggggtcgggggagtattgaaaactgggcggaggcttcccagagccggagccttgagcccgggctagtgagccggcctacagatggtcgagagaaccagctaggttccgccccggcgactgcccgcctccttttaagcacctcccgcccaacctcagctccctctggctttgccacgcttcttccttccccgccttccttgactccccgactgcactggttcctgtggccatcccgcagcgccagtccaggtgccatggctgctatgtacctccccggcctgcggcttagctggcacaggctgaggccctggtgccggtcaccatgccgtagcatccaaaccctgcgcgtgctcagtggagatctgagccagctgccggctggggttcgagactttgtggagcgcagtgcccatctgtgccaaccagcaggcatccatatttgtgatgggactgaggctgagaacgctgccacgctggccctgctggaagagcagggtcttatccggaagctccccaagtatgagaactgctggctggcccgcacagaccccaaggatgtggcacgggtagaaagcaagacggtgattgtaacttcttcgcagcgggacacagtgcctctcctggctggtggggccagtgggcagctgggcaactggatgtccccagatgagttccagagagctgtggaccagagattcccaggatgcatgcagggccgcaccatgtatgtgcttccgttcagcatgggtcccttgggctccccgctctcccgcattggagtgcagctcactgactcgccttatgtagtggcaagcatgcggattatgacccgcctggggacacatgtactccaggccctgggagatggtgacttcatcaagtgtctgcattcggtgggccagcccctgactggacatggggatcctgtgggccggtggccatgcaatccggaaaaaaccctgattggccacgtgccggagcagcgggagatcgtctccttcggcagcggctatggtgggaactccttgctgggcaagaagtgttttgccctgcgcatcgcctctcgcctggccagggatgagggctggctggcagaacacatgctgattttgggcatcaccaaccccgcagggaaaaagcgctatgtggcagctgctttccccagtgcctgtggcaagaccaatctggccatgatgcggcctgctttgccaggctggaaagtggagtgtgtgggggatgacatcgcctggatgaggtttgacagtgaaggtcaactccgggccatcaaccctgagaatggcttcttcggggtggcccctggtacctctgccaccaccaatcccaatgccatggccacaatccagagtaacactgtcttcaccaatgtggctgagaccagtgatggcggtgtgtactgggaaggcattgaccagcctcttccacctggtgtcaccgtaacctcctggctgggaaagccatggaaacctggggacaaggaaccctgtgcgcatccaaactctcgcttttgtgtcccggctcgccagtgccccatcatggacccagcctgggaggcaccagaaggtgttccaattgatgccatcatcttcggaggccgcagacctaaaggggtaccactggtgtatgaggccttcagctggcgccatggggtgtttgtcggtagtgccatgcgctctgagtccactgcagctgctgaacacaagggaaagaccattatgcacgatccctttgccatgcgacctttttttggctataacttcggacactacctggaacactggttgagcatggagggacgaaaaggtgcccggctgcctcgtatcttccatgtcaactggttccggagagatgaagcaggccgcttcctgtggccaggctttggggaaaacgctcgcgtgctagactggatctgccgaagattaggaggagaagacagtgcccgagagactcccattgggctcgtaccaaaggaaggagccctggatctcagtggcctccgagcaatagataccagtcagctgttctccatccccaaggacttctgggaacaggaggttcgtgatattaggagctacctgacagagcaagtcaaccaggatctgcccaaggaggtgttggctgagctcgaggccctggaagagcgcgtgcaaaaaatgtgacctgaggccttaggctagcaagagcgcagcacccccatctgggtagaggatcctaaggctcacagaaaacgtgaacaatttgacattaaaatgtgtgagtgttgggaggccaagccacagacctcctcccatgctgtccgataagagatgcacactttgctacacgtgtgttgttttcacttcccatttgtcctcacgggcttccttcttgtcagtctcgatgctggctcacatagtaggaacttctggaagaaggcattattcttctgaagaaacgtaacctggtatagtggtgcataccttttttaatcccagaggcagccggacctctgggagtttgtgtccagcctgcctggtctatataacaagtaccaggtcagccggggctatataagtaaaacccagtcccccacacacgcaaaaaaaacaaaaaacaaaaaaacaaaaacaaaaaccacaaaggccacagcacagtagactgctcagagttagggttagtatttttgtcccacacagacgtacagaagcctccttctccagaggaagccactccagaaccaatttcttcctcctggtttcctcccagttgcctgggaggagccacacttctttattttttacttgagacaaggtctcatgaagatcaggctggcttcaaactcactgcagctaaagataactgtgaactcctggtcctcctccctctacctcagacaactgtgctgctagggccagctagacaactaacatttctagaacaactgtctgtattctaggctcattgtcaaaggaccattttgcagctcaaaatacagagcagggttgaacaaaataacatggcttcaaatcctagcacttggaaggaccctaatcacaaagcaagagaattgtatatagtgaccctgtctaccaaaaagaaaaaaaaaaaaaaagaaaagaaaacacaaagcaaagaagtatccagtg

SEQ ID NO: 5 - DNA, 26001 bp, Rattus norvegicus
  misc_feature (1)...(26001): n = A,T,C or G
cttccaataaggacacgttttgaacacaagtgcctttggaggacattggtttggtttggtttggtttggtttagttgtcaagtcagggtttttctgtgtagccctgtctgtcctgaaattcactttgtaaaccaggctggctttgaactcatagatccatctccctctgccttcctagtgcagggcataaagggatgcaccaccaggcccagctttggaggacatttgaaatccaagcttttctctccacacatttgaaggcctaggccagatatcacagctcccccatccctgggtctctttttatgaaatattttatcaaatagaaacattactcttcaccaggctccattgcttgttttcttagaagaagcagaaaatttaaggccaggaaccagccacaaaaaggaaatagggatgttgaaagaaaggaagcccccgcgtatatcctggtggaatgagaccaggaaagagatcctagagcttcccaaaaggagaaaggttggcagcattgagagtagccccttccatgcctgctctgacctaaccagacacttgcccccaccaggcagctgtcatatctatcctgatcctcttgtttactggaggactcaggggagaggtgggagagctaagctgctaaggggtgaagggagtcaggccaaacaagctcaggaaagctgagcccttcccggttcaggccaggtatcactcactggggaaagggttggggcatggtgggtgggctcagcagagagctgtgctgaccaggggagacccaggaggcacagagcatcccaagcaccgcagggaagacttggaggctaggaagtagataatagcactaaggacagttgcatatcagagaaaggggactagggacgagacagatgagccgtctgtgctcagtggtaggggctattcaaaccccagcactcaaacaaaaagtagaggcacatgtaggttacatatgctgtaaacacagaactggggaggccaaggccggagcagcgcgcaagttcaaggccagcttgaactacagaagaagggaggagaagaaagacaaaagaaagggagaagggaccacagaggtgacactctaaggtgtcactcaccccaacccctctgctctgcacactgattttagaatcctggaagactaggtctcagtcctaattttaattttggttttgctatgtggtccagtctttctcaaaggttgcatcctcctgcctcaccctccggagtgctggcattacagatgtgcacattgttacccttgaggtcacctctaattctcttctgtccttccctctccctgctcccctctcactgacccccttggtgtgcccactaaacataactgaccaatatctataaacagttattgtcctgggcataaaggatactcagtgaattataacactggctgtccattcatgacgtttgtagattaatggaacagaaaccacactactggttaggctactggttctcaaccatcctaatggtgcaaccctttaacagtttctcatgctgtgctgacccccagccatacattattttcattgatacttcataactcatcttggtattgttattggtcataatgtaaatatctgtgtttaccagtggttttaagcaacccctgtgaaaggatcctttgacaccccccctccaccaaaggggttgccacccacaatcactgctttagacctactcaagagtaacttctaccctgtgccttctgatttagaagatccattctggtctttctatgactgaatataccccgtggtgtgaaacatccacccttctctggcccggacccctggttcccaagaatcatactccaggtgtccagaaatttcctgtacaaagagttcaaacctgcttctagggaccacgtagactttttcccctaggattgcccttccatggctggacctcactactgatacacaagcccctccctgagtggcggccctcatctaagagttgcctctcccacaacaccacatcctggacaagagccccaaggactctaagtaaacaccaaatttaaataaacccttctataccactatgaagtgtgtatgtgtaaatagagaaaaattaaaaacaaatatgtattacttcaaatgatttatattttttaaatggaagatttaaaagatgttagtacatgacaggaaagcactctaccactgagctcttttgtggggaggcatgtggtgcaagggcaaggtttcactaagtcgtccaagctggccttgaactaatggatctttttaggtggccacccaagtagctgggattaaaggttcatatcatttggtctggatggattattcatttcacatatttaaacacagagcttgtggacaactatttgtactcttgccccaggcaagactactacatttaaaaaaaatacacacggtttggagagatggctcagcagttaagagcactgactgttctttcagaggacccaggttcaatgcccagaacccataggacaactcacaaccacttgttaactccagttccaggggatttagtgccctcttcgggccgctgcaggaactgcatgcaaatatacagtcaaaacatccttatacctaaaataaaaataaatttttaaaatcattcacacacgcattatatcttgaaattgagaaatatctaactatattacctgggctttcctgggacttcttttccatcaaagaagtggagaatatctatataaaatatatatatatataaatatatatatatatatatatatatatatatatatatatatatatatataaaatcaatcaccaggccatcctcgtgactacagatgtaacaaacccttcgaactgaaatgccataaagaagggcaggatgctgcaagattttatttcatgaagatgtaatatttgtttgtttatgtatttatccagagaaagagagtagataaaagaacaaacttcagacagttctctccttctattatgatggtccccgtgattaaacacaggttacgtattattagccttagtggcctaagccttaacttatgagccgtctctcaggtccgtgaagatgtaacctggtaggagttgttgcgggcatctttctggaataatcattccgacacccatgtggaagagaggagttgtcactgcttgaggaaggtcctgaacggcttcaaccccaggatctagcttatattctcattctatactctgaaaccacagggtggggtgagcaagtaagggttcacggaagcatgagtggcgttcagtggattgttccaggccatggcccactgcttcagctatttctccaggtccttctgtccaaggaagcaagcgaagtcgagttaggtcatgcagtgctgtagctaaatcaatcaatcaatcaatcaatcaatacctagtcattggtctctcctaccttattctacttacttccgaggaggggacagggtgcctacagaagtcacattaaactaagaccggtcaaatgactgaacctggatatgggacgagggcagggatcccaggccgcgaggctgacacagttctgaagcagacaggatgggacacggaatcacaaacctggaggccaacgaggagagcagagtgggctcagggttagaactggcgcttagacatatccaaggagttgggggtcgggggagtattgaaaactggtatctgcatggaagtccagtagagtggggtgaggcagttttcggccacagacactcagcaagggccatctagtccttccctcacactgagtttgactgccagggactctgcaggtaaacatgtagcccccagcctctgcctgtccctacttggatacaagtggtttattgggaaagactttctggaagggtgagagaaagagctagagagagggggaaaagttgaattatgttgcagatgaatcaatgtgaccacggtcgggagggggaggggctgaggtgctacaatagacctgagttaccctgagttgaggcaaggaggcaggctaccgcttccttataaggaccagacacaggacacgttgtcccaggaagggacattgcatttgccaacacagtcccctgtggccacagagtgtccttaactatgtacagagtgtccccttacctacacctctctttggcttaagtaggcacagcaaaatccttaataaccagtttcctctgccctgttttcaactctctgtggccaaacgtggcccaaaattattaagttagtcttctgggttgtttgtccatttgttttcgaggcaaggtctcactctgtagcccaggctagcctccaacttagggtgatcttcctgcctcagttcctgggtgccaggattgcaggtgtgaaccaccaagctgagaaaattcaaaactccgatactgcattgttctcactcgaatacaaagtgtccccctgggtttatgcgtggtggaagggggtgtcaatttgggggaatagttgggatctaaccagtggattgatgacttggtggatccctaatccgatagcattatcagaaggccgtgtaagtaggagtgtcgtcggggatacatcttgacttgacccctcctgcatttcttttttttttttttttggttctttttttcggagctggggaccgaacccagggccttgcgcttcctaggcaagcactctaccactgagctaaatccccaacccctcctcctgcatttcttactttctgctttctatctgtcctaaggtgccgcctcctttatttccacaccctccagtcaccacaatgatctgtcttgctttggactcagaagcaaccattggccatgatggacgataacctctgagctaatgaaccatccctgccattttgtcgtgtcagatattcttgtcaccgcactaaggaaagtatctacaaatgccgttctgagcactgacaaaatctcagagcttcttgccctgctccaggaaatgagtcaccccagtccgtgtgtccacgctgttgtgtgtactttccagttaggtcggttggtagctgtcagttagcagatggaccgttctattgtcatgttacatgtgattcctttattttatttactaatagccacaaggtgtgagagtcatgaggcaggcaaaccactctccctctgacatgtcaaccctctgatgtatcatcagatcaatggtggcctaaccctatgtcatatttcgtctcactctgtgggcatcaaatcatcccacaccatagaaagaagggtaaaataagaccctgaaagacactagaaggttaggcatggtgacacacaccattaatcccagcactaggaaggcagaggcagacagatctctgtaaatttgaggccaaccttgtctacctagtaagttgtgggccagcccatgatacatagggagaccttgcctcaagaataaaagaaactctgttccatccaataaaagaaaacagcttccttcatagtaggctgccataagtattctcttattattctctttattttctttttatttgtttgtttgagaacttactacgtagatcacgttggcctagaactcacagaaaccagcctgcctctgcctcacgagcgctaggattaaaggcacatgccacaggcaggccctgccatctgcctaaggtctgagctgactaactctgctcccattcaggccaagatccagcgctctgagttgatccaccccaacactcgctccaaccacaagctactggagttcatgaagaagctgatcctgcagaaccagagctgcaagaccaccatgactctgggcaagatacccaagaggatggttcagtactgatggtgtgtcagaaaccagaaaccttgagccaagccaatgactcattacaatggatattttcatataaagctgtttggtgggggggggatatactgtgtgacacactgaggcttccaataccaccatgatgaaggactacgcaatggagaggtggggaagatgaactagaggcgcgacagctgtgacaggtgtgagagaaggctgaggtcgatggaggaacattgtcacccagcagagttagcagcctagtatgctgcagcctggagcttacccagacccagcgccctggctctgaacaagtgacaggatgtctgtgatgaagaatgcttcatttcctggattggacctccttgaaagtcctccatgatcctaacttcccctagagaccatgctgatatcaatggtctgtactactgccccaggccatgatgaagcccaagtaccatgtggatgtccgtgatccaggctgctaccaagggccatgtctgggtccgtggtcctggtgtggctgggagccatgttgatatccatggttcatgttaccacctaacgtcacacagatgtccatggtctgtgccttgcctgaagccatgtcattgtctgtgggctgtgttgccacttgaggccatgttggtgtccatggccggttctttggcaaagggctgtattgatgtccatggtccatgatgccaccagagtccaggtggaggtccttgtcgtgtgctgatgctgatgctgagaccatatggatgcccgtgcatggtccgttctattgccaagcatctggtaaaggcacaggatctgtgttcctgctgactgtgaagggctagaaagctattttggggtggcattgttgactacaggcataagaagaatggacacagaaggcttttgtaacaactcctaccccacaacccccactaacagtaacattgtaaaaagaaagccatcagccattaagttaagagaaccagctgatattccagaggtcctgagttcaaatcccagcaaccacatggtggctcacaatcatctgtaatgggatctgatgccctcttctggtgtgtctgaagacagctacagtgtactcttaaataaaataaatctttaaaaaaaaaaaaaaggcagccaaaataactatgggagagggaagaggggaaagacagtaggagccagggggtcaaggacaccacaagaaaacccacagaaccaactaaccacaggggctcacagagactgaacccgcatggctctaacctaagccctatgttaaggttgtgtagcttgatcttcttgtgggagtcctggcagtgggagcgagagctctctgactcttttgcctgcctttgggacgtttttcctcccactgggttacctcattcagccttaatatgaagggaggtgcctagtcttagtacaacctgatatatcatgtcgggttgagatcactggaggcctgcccttgtctgaagggaaatggaagagtggatgggggaaactgtgaggagaggagggacgggaactatggccaggatgtaatttatgagagaagaataaatataatttttgtaaggatgctgaagtgtagctgtccacaatcaatggctcctgatgggagtgcaggtggggaaggactcaattttcttcagggagctggccacagggagctggccacaggagctggcctggctgcagtgactgtacgggcaacacaaactggacatttttatttcctgttttattttttcttggcttttaaaaaaaatttttttaattctttgtttcttctcttttgggggaggtcacaggggtaggggacagacatgggaggactgggaggtagatgtgatcagagtgcatgaagtaaaattccccaataaggcctactggctctctcatcatttttagcctatgtatgtcagtgggtgtatgtagcatatgtacatacattttcacgcgtgtatgcgtgtgtgtatataggtgcataccttcatttatgcacgtggaggccagaggtcagggttcaatgttttacctacttgctcaccttatgttttaagacaaggtctctccctgaacctgggactcacaaagtcatttagactgactgtccaacaagcccccaagagcctcctgactttgtctccccagccctcagatcacagtcgtgtgccacacagcccagatatgagcatgtgtgtgtgtgtgtgtgtgtgtgtgtgtgtgtgtgtgtgtgcgcgcgcgcacgcacgcacgtgtacacacaaggccagagatgagcataggtgtcttcctctattgctctccacactactttttgagaaagggtctctcactgagcctggaattcgtaggttggctagacctacagccagtgaatgagggagcttcctgtccctgcctctcagcactaggattaaagatgtctgccaccaggggctggggatttagatcagtggtagagcgcttacctaggaagcgcaaggccctgggttcggtccccagctccgaaaaaaaaagaaccaaaaaaaaaaaaaaaaagatgtctgccaccaaacccagatttttacctgggttctggggatccaagttcagtttctcatgtttgcttgggactttgaccatcatcctcagtcccttactgctttaatatcaatcacttactatgaatacttacatatcaaattttatcatagatatatgtctgtgtaagaaaaattgcaaatgtatatggaattgaatacttgctaagtaatggttcctttcctctctccctctctctctctctctctctctctctctctctctctctctctctctctctcgatggaagggttttcaaaatagggtagcatcagctgtcccagaactctgtagaccatgctggccttgaactcacagagatctgcctacctctgagtctggagtcctgggattaaaggctcaggccaccacacccagctactgtgagagatgttttcactatctctcttagataaattgggactgttctcaactccagccaatctgtgaggcatccagggaataaaccttgattgtccctaagaaggaaaaaacaaacaaaaaaacgaaccagagatcagagcaaggcttggtcacagcccgccccctggtggtactgctttcacacagcgccagctggcggcgcaggctggagttgctggggattgaggagcaggcagggagctgaggaagaggctggggattgagaagcaggctgaccaagtcgggggcaagcaacgatagtggggaaaatcctacagcatgtccttgctccgtctccctaagttaggaatgaagttgtgagggattggggatttagctcagtggtagagcgcttgcctaggaagcgcaaggccctgggttcggtccccagctccgaaaaaaagaaccaaaaaaaaaaaaaaaaaaaaaggaatgaagttgtggggaagaactgcagaaatgcaggtcttgctgggtgtgggctacgtacatatataaattccagctctcgggaggcaaggcgaacctgttctacacagcaaatccaggccaggcaggctgcatagagagaaactgccccctcccaaaaaaagaatgcgggatcattcattctttcatacaatcaccattggtcaaatgactattgtaggaggacactggtaggggacagtttaaagcaagattcagtttgcttagattcttctccctagaaagcagagaaatggcagttaggcaagaaaggaggtggaggtaatcagatagctaggggccagatgagggactttaagcctatgcctagtctaagattcttcggattcctcctttttccccatttgttttttgagatagggtctagctcacaataaactaatgaaatagtataaaaaagggttgcagggaagaaaacaagacctggataactagagtcagaaagactcggagccaagttccatttctccagtcaaccagatgtgcaactctgagcaaaagacttaaccttcagtgtctacatctgcaagataaagctactgtggactgaatgatatgtgtgccccagctgctagcacagaacttatcacaccataaataccaatcctttctctatacaaggtgacaccgtacacaaagacagatggggcattgaaaggcacagttcaagaagggccttagaagtaatcaggtaaatccaggtgaaagataagctaaagtggggctaattgggtagaaccattctaaaggtttatatgagcacactgtagctatcttcagacgcaccagaagagagcatcagatcccattacagatggttgcaaggcaccatgtggttgctgggaattgaactgaggacctctggaagtgctcttaaccactgagccatctctccagtggtcctggtatggaattagctatggaggattagaaaaaaatcctttagtctgactgagatgcccccaccccggggcattgtggtagatgatgataccattcactgagataaggaatcagatgaaacaagattgtaaacaagatgactttcaaagggtcagccattctgacaccagcgtgaacaccctgagcctaccatgtagtcaaggagtaacatggggatggcaaggtgaatgtggacagttttgatgcaggaaccagaggacggggtgtggcaggtagagggtacaggtctatatgcatgaaattaaatgcaaacgtaatggggatgggaaacaccggaagccagctacagtgttggccttagaggccatctctctctttatacgcaaaactctgaggagactaaaaatcaagattatttgttttcaaagtggcagaatccgcttaaaataagcttaaatgaaagaatagattcacccattcttgtaacaagaatataggctttctgtaccacctagttccagaaacagccactatttcatccacaaagtgccaatctaagacctgagacctgatagtaaaagttagaaaggatgaatttggttgtattggtacgaggtcaccaaggctaccttcaactctagtacaggagatattgatgacatcagtgacccctcaattgacctcaactacatggtctatgtatcccagtatgattattcccatggcaagttccatggtacagtctgggagacaagtggaaaatttcccaggagttagatctctccaacatcagcggggtggatgctggtgcccagtatgctgtggagtctacagatgtcttcactgtcatgaagaaaggtggggtgactggaaggatggagcccaaagggtcatcatctctgccctctctgtttgtgatggtgtgaaccaggagaggtacaactcattcaagtttgtcaccaatgtctcccacaccatagtctgctcagcaaaccccaggccaaggccacccatcaggggatttattatcaccatcctctagaagaccttggatggcctcctggagactttgtgtaatggccatgggctacccaaaacttcccttcattcctggtacacatttaagtcctcggccacgactgcggacaaaggtcgtgcctgagctgaacagggaactcactaccatgagcttctcttcctacttccactctgcatcctggatctgacaagctgccgggagaaaactgtcatttgggagacaacaagaggtggtggctgggtgtggtgacttcccagcacttggaaagcagattagatctctcaaggacagtttcgtctacatagtgagttagttccaggacagccagactacatagggagaccttgtctccaaagtaaatacgtacgtgcagatatacatatataagaaggtgatgaggcaggcgttggagggctcctaaggggcatcctaggcttatttagggaccagttgcctcttgagactttaacagcaacatccattctacttttgatagtggggttagcattgttctcaataaccactttatcaagctcgtgtccaggtatgacaaataacttggctacggcaacagagtaatggaccatttaggtccacacagtgctccaaggtccacatgacctccaaagaataagaaaccctggaccacctgacccagcaagaacaggagaggaagagagccctcagctgttagggagagcctgctctaactcaagccccactacactgcccctcccagtctccatcttaactcccaatggaaggggcttcagaagcccttagatgcctcaatagagtccactgtgggaggctaggggacatctactgatcttcatctccctgctctgctcctgtctcaggcaggttcgtgttttgtgttaccaagccaacatccaacagagcccaacttagactctacgggctcagcgatccagcagaacatgagctcatgtccaaagtttagaaaaaatctcagacagggatctcatggcccatctctgtgcatcctgatggctgtggtcagggtgtcagccacatgaactgtccctaagagaagcaaggtcagacccacatgacctgatggttctccaaagagagtctcagtgttgtcaccagaaagaggattctgagcagctgttcgccgtggatgtgttgagtttgaattgctgcatcatttggattggagataaccaatcaagttggagtgacatgttgagaaattacacgaggactttatgatagtggcaagataagggcaaatacgctctttggaattaggtggcggtgggctagggtatgtgggagggattagaggaccaaggatgtgtctgtgagcctgaaagacccaatttcatggtgctagaggaaactcccttgataagctggagggatggagacacccactcatctccaagtgtttaacccagaattgctcctgtcggaaggaaatatggggacaaagtgtggagcagagactgaaggaaaagagactgccccacctggggatccatcccatatacagacaccaaacgcaggcactattgctaaaaggtgcatgctaacaggagcctgatacaactgtctctgagtggctctgccagagcctaagaaaaacagaggcagatgcttgcagccaaccattgaatggagcacggagaccccaatggaggagttagggaaaggactgaaggagctaaagcggtttgcaaccccattggaagaacacaatatcaaccaaccagagctcccagagctcccagggactaaaccaccaaccaaagagtacacatggagggacccatggctccagccacatatgtagcagaggatggcattgtccagcatcaataggagaagtccttggtcctgtgaaggctccgtgccccaatgtaggggaatgccagagcggtgaggtaagagtgggtaggtgggtgagagacggtataaggggtttgtagaagggaaaccgggaaaggggataacatttgaagtgtaaataaagaaaatattcaataattaaaaaaaaaagaagctgggcaaggacaactgtttggttctgttagtatttgaggactaaggagagcttagcatctgggaccctggaaaagaaaagcctaaggctaggagatctctggaaaagcaaacctagtccctgaggttttaggtgtcttgttatcctgagctccccacccctggtctcatccctcattcttagcttgtttgacatttgacgtctcctaattgccttctctaaggagcaacagccttcaaagttacatcagtatccttgcagcgtggcgggtcgtagggcacagcctcggagtgggcggatcccaagctgaactcaggcctccaatggcttgagggtgtgatgaccgtagggcggaggcttcccagagccggagccttgagcccgggctagtgagccggcctacagatggtcgagagaaccagctaggttccgccccggcgactgcccgcctccttttaagcacctcccgcccaacctcagctccctctggctttgccacgcttcttccttccccgccttccttgactccccgactgcactggttcctgtggccatcccgcagcgccagtccaggtgccatggctgctatgtacctccccggcctgcggtgagtgacccatagcacggggcccactccgcgctccatcttcggggcttcctccgcttgtttcccatcctagcaggagactggcagggcggctgttcaccagtctgggtctcatggtggtgcatccttccccacctcttcccggtcccatccccagccgtgccaggtttcccacccagcctatgtgctctccccccacctcacctcgcccactggtgcgctgttttctctgccactcgcaacctcctcccccttctctgccttcctgagcctctgcaaatgctctacttaccggatgtaattgtttcagactgggggctcccagtccacccttcagtgtgagaagccattaagggcttgaggccatagggcttgaggccccagggtgccgagctcaaagctccctaaagaaggtagaaggtaaacatcatttgcagcctccctgagaacggaacctggcttgacgcccagtgaagcctctggggactaggagggagggagagaaggaaggaagcaagatgggagaaggaaagcaaactgccttctttttaacttggctggcttcccctggtaggtcctggacccccagacttgtcctggctggcgccccccccaaccccctacaaccgggtctcctctctacgcccctttctccatggtctggggcatagagtctgagctgactccttatctctcccgcgcaggttttacttggttagtccatgtgagagtggcctgtgcaggatagagagaaggtccttcttagacttagagacctgcaggagaatgggggtgggcctgggagaagctgtgcttcacctaagacaactggagcaattcccggagggcaaaagctttcaagaccatgtctttaaaattaatttctcagggaaactctcttgggcagaaaaacacacaaagagacctaccttgctgtgcccccatgccctggggccacacaaaccatgctgcctttttttttgtcttctggaaaacaccccacccaggcccaaactcctacatccaagagcttcatttttcccccttcccaggcagtgtagcctgtggtctgggcttcccgtcctcacttgttctgaccccaccaacaacccctctcttaggagagccagagaattcacagactcaacccatcatggcctactatgggtttctgacaggtgaggcctcacacatgaaaagggtagctgtgtctctcttcgatggtggcctttgctattttgggtccaggcccatcacagcagaccctaccagcttccaggagcatagcaggaaaaaaaaacgacctaagagaccctgcacttctggaaatctaggcatggacccttagccagtggcacgattcttcttgatccacctctcgcttagttaaaacatccttatctaatccacccttgttgacccagatgaacggatgcatggcctgaaaccccaatatcagttgtcttcatgcgagaaccctaacggtaaagcctagcattggaagagggctttaccatctccagctatgcctttcatttgagacctttccaccagaggagaaagacgagaatagctgtcgttttaacataaaacctgaagggaaagaacatgcacgagcttcttaaagaccacttatgaagctctcgtttgagcaatttcttcactcttgggccagaagcttgactacctctcctccctgataggagatccatacctcaaaaaggccacaaatacaagtagcctgaactcattccacataccaatcataacctggtctctcggtgtctgcgaccagacacgcattctccagaggcatgtctttctgcccctggccgaaacttgactcccatgatagccactggttggttgcctccctgttttgacttttataggcttagctggcacaggctgaggccctggtgccggtcaccatgccgtagcatccaaaccctgcgcgtgctcagtggagatctgagccagctgccggctggggttcgagactttgtggagcgcagtgcccatctgtgccaaccagcaggcatccatatttgtgatgggactgaggctgagaacgctgccacgctggccctgctggaagagcagggtcttatccggaagctccccaagtatgagaactggtaagccttaaaactggcaacccccaagatggttactacttggtctgaggtctccgtgggttcaccacgacgaagatatttctagaccctaccagtggaatgggccagaatgggagcttcaggggaaatggagtagcaagtgaatgttttcacattaaataaaaacattgatatgaatttacatattggagctctgattttggaaggggcctgcccactgcttacctcctacttcatgaaacattttccaggggtagacactcctaggccgtgccttggagaaaatatctcttaatgaaataaagcctaaaggatctaaactgcagtcgggtaagaaaaattaaggccagcagatctggagtcaaagttggacttaaaatgtaaatggccgggggttggggatttagctcagtggtagagcgcttgcctagcaagtgcaaggccctgggttcagtccccagctccgaaaaaaagaaagaaaaaaaaaaaaagtaagtggccaggcttagaggagctggcctttaaccgcagagggcaggtgtatctctgtgagtttgaggctagcctggtctacatagtaagtttcaggacagctggagttacatggtgagaccctgccttaacaaaaatgtaacaacaaaaaagtaagtctggggataagtgaaatctgctagccaccacttcccggcaatcctctctcccccagctggctggcccgcacagaccccaaggatgtggcacgggtagaaagcaagacggtgattgtaacttcttcgcagcgggacacagtgcctctcctggctggtggggccagtgggcagctgggcaactggatgtccccagatgagttccagagagctgtggaccagagattcccaggatgcatgcagggtaacaagtcaggaacatagaggcaggggcactgaagataggactgggtttggaacccttcctcctagcgacatcttcctccgcaggccgcaccatgtatgtgcttccgttcagcatgggtcccttgggctccccgctctcccgcattggagtgcagctcactgactcgccttatgtagtggcaagcatgcggattatgacccgcctggggacacatgtactccaggccctgggagatggtgacttcatcaagtgtctgcattcggtgggccagcccctgactggacatggtaagtacctatctaggacactgaaaccttcttccactcagagggagccccaaaccttgcccacccacagagaaattgtggatgtgaacgattgctccccaaggcccagggaagcattctggactggagcgacaccgtttggcgaacagaagtcagtccggggtctgacagcacagaggctgatgggtactgaagaagggggcttagctacctctgggcgttccattgcttccacgtgtctcttgagtcagtccttgatcccttcagtccctggcaccctggtttctgatgccggtgcgggaaacactatgaccccattgtctccaggggatcctgtgggccggtggccatgcaatccggaaaaaaccctgattggccacgtgccggagcagcgggagatcgtctccttcggcagcggctatggtgggaactccttgctgggcaagaagtgttttgccctgcgcatcgcctctcgcctggccagggatgagggctggctggcagaacacatgctggtgagggcctggggaggaactgcgaagccgtggaaaggggaatgggcggggaagccttggcactctacctcagccttgcctccttcctgctaggtgccaggatggggagcctgaaacctgacgttttagccccaggctgctaaagtgccatcccagcccttgtggcagggcaggacacctgcttagtggataaacattagtgtccgtttcctgctgcctaccccatgacccgctagctgtccgtatccgtgcagaatacgccatgagtcttggtgacttggttctattccctctgtctcccaacacagattttgggcatcaccaaccccgcagggaaaaagcgctatgtggcagctgctttccccagtgcctgtggcaagaccaatctggccatgatgcggcctgctttgccaggctggaaagtggagtgtgtgggggatgacatcgcctggatgaggtttgacagtgaaggtgatgggcccttttcctgagcagacaacatggtcccatacctcgggtagcctagcaactgcagatctcccacatcagaaaaaaaaaaaaaaaagatagaggaggacttcctccagcctcacagcttagcctttcctagttggtctttcctttcaggtcaactccgggccatcaaccctgagaatggcttcttcggggtggcccctggtacctctgccaccaccaatcccaatgccatggccacaatccagagtaacactgtcttcaccaatgtggctgagaccagtgatggcggtgtgtactgggaaggcattgaccagcctcttccacctggtgtcaccgtaacctcctggctgggaaagccatggaaacctggtatgtggtcccagggctcagcacttttttagaatatggacgtgtgttctgccgacaagtgcgtctgcaccctatgcgtgtagtaaacaggctatcagatcccctggatttggaattacagatggttgtgagccaccatgttgggacttgaacctgtgtcctctgcaagagccgccagtctccagccacaacaattttaatgacagaaaactctctaccaccctagcttttggtagtttccaacaccaggcaagatctcagttcaggtccctgatccattcattcatcattgtttggggatctgagtaattgttgaacgtccctttcagttttccagttttccatccaattgaatggggtttatcttggctctcatccactaggatgtggagnnnnnnnnnnnnnnnnnnnnnnnnnnnnnnnnnnnnnnnnnnnnnnnnnngaggaattgggcaagtatcatttgacccattgggaaagaaggaccctgtctcctctaccgacatctcctgtgttcccctgtataatagcctgtgtctaaagttgtcctacccttctcatccctccctatgttgtaatccttcatgaactcaggcactttattttagaggctgcttttcccaagcctgtcattaccagggtgcctaagagacccatccctccgccgccatccgtctgccttctgcagttaaaggagaagagcactgtggaggaagagcttgtggccactgaagcacagacttgagttttgcaggctggcctcatagtcagcctccttgtttcattagcctttattttccctgacactatcattaagtcgccctcagattccccatccaactgagaaatttaaaaaaaaaaaagataatcttccctacaggctagcttcccaaccctcccatcaactctgaattaactgatacaactagagagtcacggtcctaggctgggatagacgtgtcagcctatggctcccactccctacgtccaatctggtcgtcttcttccactggagaaggcaccatgcctgagcccccccccacccagtggtgctcacacatgagctgctaacttggctcccagcttctcaccagcgtacctgaaggtaccaaatgctctgggctgctcagcttttgactgaccatgttacgttcctgattgacttcatgccctaaccaaccccattctgttagggtccaagcaagaccggagaaatcaccaactgtccgttagccttaatgtcaatcactggactggtgagatgacgcagcatgctaaggtccttaccaccaagtctgaggacctgagttagattcccagaacactcatggagaaagaaaactgaacaaggagtcctctgatttccacaagcatcccgtagcaacccctgccccccaacacaatagacaaataaataaatgtgtaataaaagcaatttctgttggcattttgatggctcaggacattgtcttcctggttagttcataggaagccatcgtttaaaatattgacctgacatgctcagagttttatgttaggtttcagaaagttagatttggtaagtaataatcctgtagctaaaagtttctgggaggttggggatttagctcagtggtagagcgcttgcctaggaagcgcaaggccctgggttcggtccccagctccgggggtgggggggagacttggccaatactaagtacccaaaatgcccttgacccatcccaacccttcttccacagctctctagcagccccattactgataaatagcctgacctttgtgggaggatgttctgaccccttctcccttctgctttacgacacgaggttgtaattagctgacgaacccatgctggagatagatccccttggtagcgtacttgccctctgtgtgtaagaccctggggttaagtttcagcatcatgaaagaaaagctcaaagaaagtgtgtgtccgtgttgaaagtgggcataaccttggaagtaatggcattttgcattttttctgccaggggacaaggaaccctgtgcgcatccaaactctcgcttttgtgtcccggctcgccagtgccccatcatggacccagcctgggaggcaccagaaggtgttccaattgatgccatcatcttcggaggccgcagacctaaaggtaaacagtgtgggggggggggcactgtgcctttgtagtaaagtccagctctacagtcaagctcccacctccctgtacttaaaactcacaggaccgctcactcgggacctccagctgctgtccaaccacgtctcccatagtgaatgcagaatgtgcgagggtgaggggtctgaaaatgggagaaggaaattgtctgtaaacctgttcattggtgttccaggggtaccactggtgtatgaggccttcagctggcgccatggggtgtttgtcggtagtgccatgcgctctgagtccactgcagctgctgaacacaagggtaagtttccaccatccactgttccccgcccctgtccactgtttcccttccgccgatcccctctccttactctcccggaagctttttcagaactgttaacttttttttcattttttttttttgtattttttttcatcctcgagtatttcttatttacatttcaattgttattccctttcccggtttccgggccaacatccccctaactcctcctcctcccctactatatgggtgttcacctccccatcctccccccattaccaccctctccccaacaatcctgttcactgggggttcagtcttggcaggaccaagggcttccccttccactggtgaccttactaggctattcattgctacctatgaggttggagcccagggtcagtccatgtatagtctttgggtagtggcttagtctctggaagctctggttgcttggcattgttgtacatatggggtctcaagccccttcaagctctttcagttctttctctgattccttcaacgggggtcccgttctcagttcagtggtttaatgatggcattcacctatgtatttgctgtattctggctgtgtctctcaggagagatctacattcggctcctgtctgcctgcacttctttgcttcatccatcttgtctaattgggtggctgtatatgtatgggccacatgtgggccaggctctgaatgggtgttccttcagcctctgttctaaactttgcctccctattccctcccaagggtattcttgttcccctgttaaagaaggagtgaagcattcacattttagaactgttaactttttacccaacctctgacagctggacacaggacactcttcccttcctgagaaaggaaggttccttacccaccctcctctctgtcctaggaaagaccattatgcacgatccctttgccatgcgacctttttttggctataacttcggacactacctggaacactggttgagcatggagggacgaaaaggtgcccggctgcctcgtatcttccatgtcaactggttccggagagatgaagcaggccgcttcctgtggccaggctttggggaaaacgctcgcgtgctagactggatctgccgaagattaggaggagaagacagtgcccgagagactcccattgggctcgtaccaaaggaaggagccctggatctcagtggcctccgagcaatagataccagtcagctgttctccatccccaaggacttctgggaacaggaggttcgtgatattaggagctacctgacagagcaagtcaaccaggatctgcccaaggaggtgttggctgagctcgaggccctggaagagcgcgtgcaaaaaatgtgacctgaggccttaggctagcaagagcgcagcacccccatctgggtagaggatcctaaggctcacagaaaacgtgaacaatttgacattaaaatgtgtgagtgttgggaggccaagccacagacctcctcccatgctgtccgataagagatgcacactttgctacacgtgtgttgttttcacttcccatttgtcctcacgggcttccttcttgtcagtctcgatgctggctcacatagtaggaacttctggaagaaggcattattcttctgaagaaacgtaacctggtatagtggtgcataccttttttaatcccagaggcagccggacctctgggagtttgtgtccagcctgcctggtctatataacaagtaccaggtcagccggggctatataagtaaaacccagtcccccacacacgcaaaaaaaacaaaaaacaaaaaaacaaaaacaaaaaccacaaaggccacagcacagtagactgctcagagttagggttagtatttttgtcccacacagacgtacagaagcctccttctccagaggaagccactccagaaccaatttcttcctcctggtttcctcccagttgcctgggaggagccacacttctttattttttacttgagacaaggtctcatgaagatcaggctggcttcaaactcactgcagctaaagataactgtgaactcctggtcctcctccctctacctcagacaactgtgctgctagggccagctagacaactaacatttctagaacaactgtctgtattctaggctcattgtcaaaggaccattttgcagctcaaaatacagagcagggttgaacaaaataacatggcttcaaatcctagcacttggaaggaccctaatcacaaagcaagagaattgtatatagtgaccctgtctaccaaaaagaaaaaaaaaaaaaaagaaaagaaaacacaaagcaaagaagtatccagtgaatggtcccgaatctttgtgtatatgggcagcagggggttattataaaaaaggaaaagattggaagttcagagggaaatgggaaataggacaaggggagtggattggaccaaatgacgtagatacataaatttcccaataataacactgtgaggtactttttaaataggctgttggaaaaatggctcaatagttaagagtgtttactgctctagaagaggactcgagttcagcagccacactgggcgggctcagctcctgcagctccaggagatctgacaatcctgacattcacaagcccctcatatgcacatactcaatctttgaaaaagaaaagtggcctaggatgtaacccagtgacaggttccatttccctgcgcagcagaaagcaaaagaacacaggccaagtaacttgtcaaaggcaaaatcataagcaggtagagtcacctctctaaatcccctggggaccagatccagctgcacagcctcacccactctggggcagtgagctgagcatggtggcacacaccttcagtcccaggactcgggaggagttctggcacagccaggatgacaaagaaattccctgactctaagaagagaaaaagactctacatgatactaatctttggaataaagttcattcggtagcaagataaccacttagtttctgaaatagattatgagggactgaaaaagctcagtaactgtctcctccactttcagagccccagaattcaggtcgcagcacccacaatgcgtggcttaccacctataaccctggctccaagtctttgagggttccagtacatatatggcatacactcacatagacacatgaatttagatttaatttttgtatgtatatcaatgtagctgcctgaatgtgtgcacaccacatgtatacttggtaccatgaaggtcagaattggatgctctggacctgtgagacaacatggatgctgggaactgaactcgagttctgctctgatagagcaagaagtgctctaagccactagccatctctctagctcattaataagatgttagaaagtaaaacaggagcaaccgtcccacgtgaactggagcgtttgctaaggagaacgtccctgcttaggaacaagatgaatatatgaacttagaaaatgaagaaaatcttcaaataaaattggattcctggaaaagccttcagaagaagcaactggatgagaagatgctgagtgtcctccaagaaaattctggaaaactggccagggaagaaaggttcactaaaggtcagagtggaccacaagatagaacacagcaatagaatacacatcccataaaaatacaactggtagaagaggcggggagggttgatcagatcctgagttacagcaaggttatagcaactaaggttgcttaaggataacatcacaaaagagctgaagggccaggtgtggtgacaccggcctttaatcccagcactcaggaggcagaggctagagaacctcagagttggaggccagcgtggtctacagagctagttccaggataacaaaagctgaggcctagtctcaaaaatacaaaaacaaaaacaaaaaaaatacaaaaacaaaaacctgatagatttttaatgctttccagttttagcacaaagaaatgatgtttgaaaggaagttcatcctaatttgaacattacacagtacatatgtgcatctaaacatcatgtgatacctcacgaatatgcataatcttatgtcaaaagaaacggtataatttaacagtcgatgctcatcttcataagcagcgatgggtaactgacacgatgccctttgcacacagtggctctaccccttcatctgtaaaatgggaacatacctcagtgtcgttgggatgaccgagtcagtagagtaaagcactccagctagcagcaaaaaccaaaccaacactctagacagcctcagctcctctccacgccaatgattagagttgggcctttaagtccgtcttatatagccgactctgccgttaagagggaaaagagcagtgaaaagaaaacataggagagggttggggatttagctcagtggtagagcgcttgcctaggaagcgcaaggccctgggttcggtccccagctccggaaaaaaaaaaaaaaaagaaaacataggagagaagaagattcagaaatgggaccctccaggaaagaggcaggggaacttgagccccagatacctcttgcatagcaccaagccttaaatatgaataaaattggcccacgtggcagataagagaagcatgattaaaattcttttgagttttagtcttgttcgcttgcttgctttgggttttgtttggagtttgtatgtttgcttgagacaaggtttctacagcccagattggtgttacagaaaacatgttacagaagttagtagtgaattcctgatcctcctgcctccacctcccaaatgctggggttacaggtgtgttaccatgcccagccttggctttagatttggttccctcccccttcccattctttcttcttctcagttttgttttacctgcaataccagagtggaatctagggccccaggaaggccagcaagtggcctaacattgacttacattccaggttg

SEQ ID NO: 6 - DNA, 21 bp, Artificial Sequence
  Primer
agaccctgcgagtgcttagtg

SEQ ID NO: 7 - DNA, 20 bp, Artificial Sequence
  Primer
gatgtggatgccctctggtt

SEQ ID NO: 8 - DNA, 26 bp, Artificial Sequence
  Probe
ccagcttcccactggcattcgagatt

SEQ ID NO: 9 - DNA, 20 bp, Artificial Sequence
  Synthetic Oligonucleotide
aggaaccgagcggagccggg

SEQ ID NO: 10 - DNA, 20 bp, Artificial Sequence
  Synthetic Oligonucleotide
tgcggccatggcacctgggc

SEQ ID NO: 11 - DNA, 20 bp, Artificial Sequence
  Synthetic Oligonucleotide
ggcggtacaatgcggccatg

SEQ ID NO: 12 - DNA, 20 bp, Artificial Sequence
  Synthetic Oligonucleotide
gctacggcatgatggccagc

SEQ ID NO: 13 - DNA, 20 bp, Artificial Sequence
  Synthetic Oligonucleotide
aatgccagtgggaagctggc

SEQ ID NO: 14 - DNA, 20 bp, Artificial Sequence
  Synthetic Oligonucleotide
tccatcacagatgtggatgc

SEQ ID NO: 15 - DNA, 20 bp, Artificial Sequence
  Synthetic Oligonucleotide
tcggatgaggccctgctgct

SEQ ID NO: 16 - DNA, 20 bp, Artificial Sequence
  Synthetic Oligonucleotide
caccgtcttgctctctactc

SEQ ID NO: 17 - DNA, 20 bp, Artificial Sequence
  Synthetic Oligonucleotide
ctgcatgcagcctggaaacc

SEQ ID NO: 18 - DNA, 20 bp, Artificial Sequence
  Synthetic Oligonucleotide
gagctgcaccccgatgcggg

SEQ ID NO: 19 - DNA, 20 bp, Artificial Sequence
  Synthetic Oligonucleotide
ccagtcgggtcataatacgc

SEQ ID NO: 20 - DNA, 20 bp, Artificial Sequence
  Synthetic Oligonucleotide
cacttgacaaagtcaccatc

SEQ ID NO: 21 - DNA, 20 bp, Artificial Sequence
  Synthetic Oligonucleotide
ttgcacggccactggctcac

SEQ ID NO: 22 - DNA, 20 bp, Artificial Sequence
  Synthetic Oligonucleotide
tgatctcccgctggtcgggc

SEQ ID NO: 23 - DNA, 20 bp, Artificial Sequence
  Synthetic Oligonucleotide
cttgcccagcagggagttgc

SEQ ID NO: 24 - DNA, 20 bp, Artificial Sequence
  Synthetic Oligonucleotide
ccgggccagccgagaggcga

SEQ ID NO: 25 - DNA, 20 bp, Artificial Sequence
  Synthetic Oligonucleotide
ggtgatgcccaggatcagca

SEQ ID NO: 26 - DNA, 20 bp, Artificial Sequence
  Synthetic Oligonucleotide
ggcactagggaaggcggctg

SEQ ID NO: 27 - DNA, 20 bp, Artificial Sequence
  Synthetic Oligonucleotide
tccagcctggcagtgcaggc

SEQ ID NO: 28 - DNA, 20 bp, Artificial Sequence
  Synthetic Oligonucleotide
ggcccggagtcgaccttcac

SEQ ID NO: 29 - DNA, 20 bp, Artificial Sequence
  Synthetic Oligonucleotide
gcgttgggattggtggtggc

SEQ ID NO: 30 - DNA, 20 bp, Artificial Sequence
  Synthetic Oligonucleotide
agtacacgccaccatcactg

SEQ ID NO: 31 - DNA, 20 bp, Artificial Sequence
  Synthetic Oligonucleotide
tttccagggtttgcccagcc

SEQ ID NO: 32 - DNA, 20 bp, Artificial Sequence
  Synthetic Oligonucleotide
gggcctcccaggctgggtcc

SEQ ID NO: 33 - DNA, 20 bp, Artificial Sequence
  Synthetic Oligonucleotide
cccacaaacaccccatgacg

SEQ ID NO: 34 - DNA, 20 bp, Artificial Sequence
  Synthetic Oligonucleotide
ctttgtgttctgctgcagca

SEQ ID NO: 35 - DNA, 20 bp, Artificial Sequence
  Synthetic Oligonucleotide
gtagtgcccgaagttgtagc

SEQ ID NO: 36 - DNA, 20 bp, Artificial Sequence
  Synthetic Oligonucleotide
tcacgccggaaccagttgac

SEQ ID NO: 37 - DNA, 20 bp, Artificial Sequence
  Synthetic Oligonucleotide
gagcattctccccaaagcct

SEQ ID NO: 38 - DNA, 20 bp, Artificial Sequence
  Synthetic Oligonucleotide
tgggtgtctctcgggcactg

SEQ ID NO: 39 - DNA, 20 bp, Artificial Sequence
  Synthetic Oligonucleotide
tgaggccgctgagatccaag

SEQ ID NO: 40 - DNA, 20 bp, Artificial Sequence
  Synthetic Oligonucleotide
tcacgaacctcctgttccca

SEQ ID NO: 41 - DNA, 20 bp, Artificial Sequence
  Synthetic Oligonucleotide
ggcagatcctggttgacctg

SEQ ID NO: 42 - DNA, 20 bp, Artificial Sequence
  Synthetic Oligonucleotide
tcacattttgtgcacacgtc

SEQ ID NO: 43 - DNA, 20 bp, Artificial Sequence
  Synthetic Oligonucleotide
tgccttccctattcccagat

SEQ ID NO: 44 - DNA, 20 bp, Artificial Sequence
  Synthetic Oligonucleotide
aagatgttagttaatatcaa

SEQ ID NO: 45 - DNA, 20 bp, Artificial Sequence
  Synthetic Oligonucleotide
ggacagtctttgtgggaagg

SEQ ID NO: 46 - DNA, 20 bp, Artificial Sequence
  Synthetic Oligonucleotide
accaggcgggagtggtaccg

SEQ ID NO: 47 - DNA, 20 bp, Artificial Sequence
  Synthetic Oligonucleotide
agactaggcctcaggtcaca

SEQ ID NO: 48 - DNA, 20 bp, Artificial Sequence
  Synthetic Oligonucleotide
ttaaaatagataagcatctc

SEQ ID NO: 49 - DNA, 19 bp, Artificial Sequence
  Primer
tgggaaagccatggaaacc

SEQ ID NO: 50 - DNA, 16 bp, Artificial Sequence
  Primer
gcgagccgggacacaa

SEQ ID NO: 51 - DNA, 24 bp, Artificial Sequence
  Probe
acaaggaaccctgtgcgcatccaa

SEQ ID NO: 52 - DNA, 20 bp, Artificial Sequence
  Synthetic Oligonucleotide
cactagcccgggctcaaggc

SEQ ID NO: 53 - DNA, 20 bp, Artificial Sequence
  Synthetic Oligonucleotide
gtaggccggctcactagccc

SEQ ID NO: 54 - DNA, 20 bp, Artificial Sequence
  Synthetic Oligonucleotide
gggcggaacctagctggttc

SEQ ID NO: 55 - DNA, 20 bp, Artificial Sequence
  Synthetic Oligonucleotide
gctgcgggatggccacagga

SEQ ID NO: 56 - DNA, 20 bp, Artificial Sequence
  Synthetic Oligonucleotide
cagccatggcacctggactg

SEQ ID NO: 57 - DNA, 20 bp, Artificial Sequence
  Synthetic Oligonucleotide
ggaggtacatagcagccatg

SEQ ID NO: 58 - DNA, 20 bp, Artificial Sequence
  Synthetic Oligonucleotide
ggcaccagggcctcagcctg

SEQ ID NO: 59 - DNA, 20 bp, Artificial Sequence
  Synthetic Oligonucleotide
ctacggcatggtgaccggca

SEQ ID NO: 60 - DNA, 20 bp, Artificial Sequence
  Synthetic Oligonucleotide
tgtgcgggccagccagcagt

SEQ ID NO: 61 - DNA, 20 bp, Artificial Sequence
  Synthetic Oligonucleotide
acccgtgccacatccttggg

SEQ ID NO: 62 - DNA, 20 bp, Artificial Sequence
  Synthetic Oligonucleotide
caccagccaggagaggcact

SEQ ID NO: 63 - DNA, 20 bp, Artificial Sequence
  Synthetic Oligonucleotide
ctggccccaccagccaggag

SEQ ID NO: 64 - DNA, 20 bp, Artificial Sequence
  Synthetic Oligonucleotide
atccagttgcccagctgccc

SEQ ID NO: 65 - DNA, 20 bp, Artificial Sequence
  Synthetic Oligonucleotide
ccctgcatgcatcctgggaa

SEQ ID NO: 66 - DNA, 20 bp, Artificial Sequence
  Synthetic Oligonucleotide
gggacccatgctgaacggaa

SEQ ID NO: 67 - DNA, 20 bp, Artificial Sequence
  Synthetic Oligonucleotide
ggagcccaagggacccatgc

SEQ ID NO: 68 - DNA, 20 bp, Artificial Sequence
  Synthetic Oligonucleotide
taaggcgagtcagtgagctg

SEQ ID NO: 69 - DNA, 20 bp, Artificial Sequence
  Synthetic Oligonucleotide
tgtccccaggcgggtcataa

SEQ ID NO: 70 - DNA, 20 bp, Artificial Sequence
  Synthetic Oligonucleotide
cctggagtacatgtgtcccc

SEQ ID NO: 71 - DNA, 20 bp, Artificial Sequence
  Synthetic Oligonucleotide
ggctggcccaccgaatgcag

SEQ ID NO: 72 - DNA, 20 bp, Artificial Sequence
  Synthetic Oligonucleotide
caggatccccatgtccagtc

SEQ ID NO: 73 - DNA, 20 bp, Artificial Sequence
  Synthetic Oligonucleotide
ggccaccggcccacaggatc

SEQ ID NO: 74 - DNA, 20 bp, Artificial Sequence
  Synthetic Oligonucleotide
attgcatggccaccggccca

SEQ ID NO: 75 - DNA, 20 bp, Artificial Sequence
  Synthetic Oligonucleotide
ttccggattgcatggccacc

SEQ ID NO: 76 - DNA, 20 bp, Artificial Sequence
  Synthetic Oligonucleotide
cgtggccaatcagggttttt

SEQ ID NO: 77 - DNA, 20 bp, Artificial Sequence
  Synthetic Oligonucleotide
tgcccagcaaggagttccca

SEQ ID NO: 78 - DNA, 20 bp, Artificial Sequence
  Synthetic Oligonucleotide
agaggcgatgcgcagggcaa

SEQ ID NO: 79 - DNA, 20 bp, Artificial Sequence
  Synthetic Oligonucleotide
ccctggccaggcgagaggcg

SEQ ID NO: 80 - DNA, 20 bp, Artificial Sequence
  Synthetic Oligonucleotide
agccctcatccctggccagg

SEQ ID NO: 81 - DNA, 20 bp, Artificial Sequence
  Synthetic Oligonucleotide
ggttggtgatgcccaaaatc

SEQ ID NO: 82 - DNA, 20 bp, Artificial Sequence
  Synthetic Oligonucleotide
ccgcatcatggccagattgg

SEQ ID NO: 83 - DNA, 20 bp, Artificial Sequence
  Synthetic Oligonucleotide
accaggtggaagaggctggt

SEQ ID NO: 84 - DNA, 20 bp, Artificial Sequence
  Synthetic Oligonucleotide
aggccactgagatccagggc

SEQ ID NO: 85 - DNA, 20 bp, Artificial Sequence
  Synthetic Oligonucleotide
atatagaccaggcaggctgg

SEQ ID NO: 86 - DNA, 20 bp, Artificial Sequence
  Synthetic Oligonucleotide
caaagcctggccacaggaag

SEQ ID NO: 87 - DNA, 20 bp, Artificial Sequence
  Synthetic Oligonucleotide
cagggcctcgagctcagcca

SEQ ID NO: 88 - DNA, 20 bp, Artificial Sequence
  Synthetic Oligonucleotide
cccaggtttccatggctttc

SEQ ID NO: 89 - DNA, 20 bp, Artificial Sequence
  Synthetic Oligonucleotide
cggcagatccagtctagcac

SEQ ID NO: 90 - DNA, 20 bp, Artificial Sequence
  Synthetic Oligonucleotide
gaagccattctcagggttga

SEQ ID NO: 91 - DNA, 20 bp, Artificial Sequence
  Synthetic Oligonucleotide
gaagcccgtgaggacaaatg

SEQ ID NO: 92 - DNA, 20 bp, Artificial Sequence
  Synthetic Oligonucleotide
gcacgcgctcttccagggcc

SEQ ID NO: 93 - DNA, 20 bp, Artificial Sequence
  Synthetic Oligonucleotide
gccacaggaagcggcctgct

SEQ ID NO: 94 - DNA, 20 bp, Artificial Sequence
  Synthetic Oligonucleotide
gccagcatcgagactgacaa

SEQ ID NO: 95 - DNA, 20 bp, Artificial Sequence
  Synthetic Oligonucleotide
gcccggagttgaccttcact

SEQ ID NO: 96 - DNA, 20 bp, Artificial Sequence
  Synthetic Oligonucleotide
ggaagaggctggtcaatgcc

SEQ ID NO: 97 - DNA, 20 bp, Artificial Sequence
  Synthetic Oligonucleotide
ggatcctctacccagatggg

SEQ ID NO: 98 - DNA, 20 bp, Artificial Sequence
  Synthetic Oligonucleotide
ggatggagaacagctgactg

SEQ ID NO: 99 - DNA, 20 bp, Artificial Sequence
  Synthetic Oligonucleotide
ggcaactgggaggaaaccag

SEQ ID NO: 100 - DNA, 20 bp, Artificial Sequence
  Synthetic Oligonucleotide
ggcactggcgagccgggaca

SEQ ID NO: 101 - DNA, 20 bp, Artificial Sequence
  Synthetic Oligonucleotide
ggccatggcattgggattgg

SEQ ID NO: 102 - DNA, 20 bp, Artificial Sequence
  Synthetic Oligonucleotide
gggtccttccaagtgctagg

SEQ ID NO: 103 - DNA, 20 bp, Artificial Sequence
  Synthetic Oligonucleotide
gtggactcagagcgcatggc

SEQ ID NO: 104 - DNA, 20 bp, Artificial Sequence
  Synthetic Oligonucleotide
tacaccagtggtaccccttt

SEQ ID NO: 105 - DNA, 20 bp, Artificial Sequence
  Synthetic Oligonucleotide
tacgaggcagccgggcacct

SEQ ID NO: 106 - DNA, 20 bp, Artificial Sequence
  Synthetic Oligonucleotide
tagcctaaggcctcaggtca

SEQ ID NO: 107 - DNA, 20 bp, Artificial Sequence
  Synthetic Oligonucleotide
tcctttggtacgagcccaat

SEQ ID NO: 108 - DNA, 20 bp, Artificial Sequence
  Synthetic Oligonucleotide
tcgagctcagccaacacctc

SEQ ID NO: 109 - DNA, 20 bp, Artificial Sequence
  Synthetic Oligonucleotide
tctcagggttgatggcccgg

SEQ ID NO: 110 - DNA, 20 bp, Artificial Sequence
  Synthetic Oligonucleotide
tgccttcttccagaagttcc

SEQ ID NO: 111 - DNA, 20 bp, Artificial Sequence
  Synthetic Oligonucleotide
tgctcggaggccactgagat

SEQ ID NO: 112 - DNA, 20 bp, Artificial Sequence
  Synthetic Oligonucleotide
tgctttgtgattagggtcct

SEQ ID NO: 113 - DNA, 20 bp, Artificial Sequence
  Synthetic Oligonucleotide
tggtacccctttaggtctgc

SEQ ID NO: 114 - DNA, 20 bp, Artificial Sequence
  Synthetic Oligonucleotide
tggtatctattgctcggagg

SEQ ID NO: 115 - DNA, 20 bp, Artificial Sequence
  Synthetic Oligonucleotide
tggtggtggcagaggtacca

SEQ ID NO: 116 - DNA, 20 bp, Artificial Sequence
  Synthetic Oligonucleotide
tgttcccagaagtccttggg

SEQ ID NO: 117 - DNA, 20 bp, Artificial Sequence
  Synthetic Oligonucleotide
ttggaacaccttctggtgcc

SEQ ID NO: 118 - DNA, 20 bp, Artificial Sequence
  Synthetic Oligonucleotide
ttgggcagatcctggttgac

SEQ ID NO: 119 - DNA, 20 bp, Artificial Sequence
  Synthetic Oligonucleotide
gcccagtgtggctgctgaac

SEQ ID NO: 120 - DNA, 20 bp, Artificial Sequence
  Synthetic Oligonucleotide
gctcactgccccagagtggg

SEQ ID NO: 121 - DNA, 20 bp, Artificial Sequence
  Synthetic Oligonucleotide
tgtgtgccaccatgctcagc

SEQ ID NO: 122 - DNA, 20 bp, Artificial Sequence
  Synthetic Oligonucleotide
agcctgcgccgccagctggc

SEQ ID NO: 123 - DNA, 20 bp, Artificial Sequence
  Synthetic Oligonucleotide
gtcactcaccgcaggccggg

SEQ ID NO: 124 - DNA, 20 bp, Artificial Sequence
  Synthetic Oligonucleotide
gacttgttaccctgcatgca

SEQ ID NO: 125 - DNA, 20 bp, Artificial Sequence
  Synthetic Oligonucleotide
acatggtgcggcctgcggag

SEQ ID NO: 126 - DNA, 20 bp, Artificial Sequence
  Synthetic Oligonucleotide
ggtacttaccatgtccagtc

SEQ ID NO: 127 - DNA, 20 bp, Artificial Sequence
  Synthetic Oligonucleotide
ccacaggatcccctggagac

SEQ ID NO: 128 - DNA, 20 bp, Artificial Sequence
  Synthetic Oligonucleotide
gggaccacataccaggtttc

SEQ ID NO: 129 - DNA, 20 bp, Artificial Sequence
  Synthetic Oligonucleotide
gtggtacccctggaacacca

SEQ ID NO: 130 - DNA, 20 bp, Artificial Sequence
  Synthetic Oligonucleotide
ccttccctgaaggttcctcc

SEQ ID NO: 131 - DNA, 21 bp, Artificial Sequence
  Synthetic Oligonucleotide
aacgtgaacaatttgacatta

SEQ ID NO: 132 - DNA, 21 bp, Artificial Sequence
  Synthetic Oligonucleotide
tcccattgggctcgtaccaaa

SEQ ID NO: 133 - DNA, 21 bp, Artificial Sequence
  Synthetic Oligonucleotide
aattctccgaacgtgtcacgt

SEQ ID NO: 134 - DNA, 24 bp, Artificial Sequence
  Synthetic Oligonucleotide
ttatgcacgatccctttgccatgc

SEQ ID NO: 135 - DNA, 24 bp, Artificial Sequence
  Synthetic Oligonucleotide
tccttcctttggtacgagcccaat

SEQ ID NO: 136 - DNA, 20 bp, Artificial Sequence
  Synthetic Oligonucleotide
gttaccagggctgccttctc

SEQ ID NO: 137 - DNA, 19 bp, Artificial Sequence
  Synthetic Oligonucleotide
gggtttcccgttgatgacc

(19) United States
(12) Patent Application Publication
(10) Pub. No.: US 2013/0217749 A1
(43) Pub. Date: Aug. 22, 2013

(54) MODULATION OF PHOSPHOENOLPYRUVATE CARBOXYKINASE-MITCHONDRIAL (PEPCK-M) EXPRESSION

(71) Applicants: Sanjay Bhanot, Carlsbad, CA (US); Gerald Shulman, East Haven, CT (US)

(72) Inventors: Sanjay Bhanot, Carlsbad, CA (US); Gerald Shulman, East Haven, CT (US)

(73) Assignees: Yale University, New Haven, CT (US); Isis Pharmaceuticals, Inc., Carlsbad, CA (US)

(21) Appl. No.: 13/702,965

(22) PCT Filed: Jun. 10, 2011
(86) PCT No.: PCT/US11/39908

§ 371 Date: May 7, 2013

Related U.S. Application Data
(60) Provisional application No. 61/353,601, filed on Jun. 10, 2010.

(51) Int. Cl. A61K 31/7088

(52) U.S. Cl. CPC A61K 31/7088
USPC 514/44 A

(57) ABSTRACT

Provided herein are methods, compounds, and compositions for reducing expression of phosphoenolpyruvate carboxykinase-mitochondrial (PEPCK-M) mRNA and protein in an animal. Also provided herein are methods, compounds, and compositions for preventing or decreasing diabetes, obesity, metabolic syndrome, diabetic dyslipidemia, and/or hypertriglyceridemia in an animal. Such methods, compounds, and compositions are useful to treat, prevent, delay, or ameliorate any one or more of diabetes, obesity, metabolic syndrome, diabetic dyslipidemia, and/or hypertriglyceridemia, or a symptom thereof.

This application claims the benefit of priority of provisional application Ser. No. 61/353,601, filed Jun. 10, 2010, the entire contents of which is incorporated herein by reference.

### STATEMENT OF GOVERNMENT SUPPORT

This invention was made with United States Government support under NIH Grants K08 DK-080142 and R01 DK-40936. The United States Government has certain rights in the invention.

### SEQUENCE LISTING

The present application is being filed along with a Sequence Listing in electronic format. The Sequence Listing is provided as a file entitled BIOL0132WOSEQ.TXT, created on Jun. 10, 2010 which is 101 Kb in size. The information in the electronic format of the sequence listing is incorporated herein by reference in its entirety.

### FIELD OF THE INVENTION

Provided herein are methods, compounds, and compositions for reducing expression of phosphoenolpyruvate carboxykinase-mitochondrial (PEPCK-M) mRNA and protein in an animal. Also, provided herein are methods, compounds, and compositions having a PEPCK-M inhibitor for reducing PEPCK-M related diseases or conditions in an animal. Such methods, compounds, and compositions are useful, for example, to treat, prevent, delay, decrease or ameliorate any one or more metabolic disease, including but not limited to diabetes, obesity, metabolic syndrome, diabetic dyslipidemia, or hypertriglyceridemia, or a symptom thereof, in an animal.

### BACKGROUND

Phosphoenolpyruvate carboxykinase (PEPCK) was first isolated and characterized by Kurahashi and Utter in 1954. The enzyme catalyzes the formation of phosphoenolpyruvate by decarboxylation of oxalacetate on hydrolysis of GTP, a key regulatory step in the de novo synthesis of glucose (Utter, M. F. and Kurahashi, K. 1954. J. Biol. Chem. 207: 787-802; Nordlie, R. C. and Lardy, H. A. 1963. J. Biol. Chem. 238: 2259-2263). The PEPCK protein occurs in two isozyme forms in vertebrates: 1) a cytosolic form (PEPCK-C, PCK1), whose mRNA levels are activated by hormones, such as glucagon (mediated by CAMP), insulin, and glucocorticoids, and inhibited by insulin (Lamers, W. H. et al., et al., 1982. Proc. Natl. Acad. Sci. U.S.A. 79: 5137-5141; Granner, D. et al., 1983. Nature. 305: 549-551), and 2) a mitochondrial form (PEPCK-M, PCK2), whose activity appears to be constitutive (Garber, A. J. et al., 1972. In Metabolism and the Regulation of Metabolic Processes in the Mitochondria (Mehlman and Hanson, eds) 109-135, Academic Press, NY).

Gluconeogenesis from lactate and amino acids is important for the maintenance of circulating glucose levels during fasting (Chandramouli, V. et al., 1997. Am. J. Physiol. Endocrinol. Metab. 273: E1209-E1215) or strenuous activity (Petersen, K. F. et al., 2004. J. Clin. Endocrinol. Metab. 89: 4656-64). PEPCK activity has been linked as the rate-limiting step of gluconeogenesis (Hanson, R. W. and Patel, Y. M. 1994. Adv. Enzymol. Relat. Areas Mol. Biol. 69: 203-281). Under pathological conditions, such as insulin resistance and type 2 diabetes, the effect of insulin in suppressing PEPCK transcription is diminished, which leads to enhanced hepatic glucose output. Increased hepatic gluconeogenesis is an important contributor to the fasting hyperglycemia found in Type 2 diabetic patients. Due to the important role of dysregulated gluconeogenesis in the pathology of Type 2 diabetes, regulation of the rate-limiting enzyme PEPCK could lead to treatment of insulin-resistant individuals.

Currently, inhibitors of PEPCK include several classes of small molecules, peptides and antisense inhibitors. Studies on inhibitors of PEPCK include sodium arsenite (Chanda, D. et al., 2008. Am. J. Physiol. Endocrinol. Metab. 295: E368-79), the ethanolic extract of Russian tarragon, Artemisia dracunculus L (Govorko, D. et al., 2007. Am. J. Physiol. Endocrinol. Metab. 293: E1503-10), 5-aminoimidazole-4-carboxamide riboside (Berasi, S. P. et al. 2006. J. Biol. Chem. 281: 27167-77), 2,3,7,8-Tetrachlorodibenzo-p-dioxin and 1,2,3,4,7,8-hexachlorodibenzo-p-dioxin (Croutch, C. R. et al., 2005. Toxicol. Sci. 85: 560-71), insulin (Gabbay, R. A. et al., 1996. J. Biol. Chem. 271: 1890-7), Loperamide (Tzeng, T. F. et al., 2003. Clin. Exp. Pharmacol. Physiol. 30: 734-8), bile acids (De Fabiani, E. et al., 2003. J. Biol. Chem. 278: 39124-32), Troglitazone (Davies, G. F. et al., 2001. Biochem. Pharmacol. 62: 1071-9), 5-aminoimidazole-4-carboxamide riboside (Lochhead, P. A. et al., 2000. Diabetes. 49: 896-903), isoferulic acid (Liu, I. M. et al., 2000. Br. J. Pharmacol. 129: 631-6), peroxovanadate-nicotinic acid (Wang Y. and Yu, B. 1997. Drugs. Exp. Clin. Res. 23: 111-5), the calcium ionophore A23187, phenylephrine, vasopressin, prostaglandins E2 and F2 alpha (Valera, A. et al., 1993. FEBS Lett. 333: 319-24), lithium (Bosch, F. et al., 1992. J. Biol. Chem. 267: 2888-93), dihydroxyacetone phosphate (Wapnir, R. A. and Stiel, L. 1985. Biochem. Med. 33: 141-8), hydrazine, phenylzine and nialamide (Haeckel, R. and Oellerich, M. 1977. Eur. J. Clin. Invest. 7: 393-400), phorbol esters (Messina, J. L. 1992. Biochim. Biophys. Acta. 1137: 225-30), cycloheximide and anisomycin (Bortoff, K. D. and Messina, J. L. 1992. Mol. Cell. Endocrinol. 84: 39-46), vanadate (Bosch, F. et al., 1990. J. Biol. Chem. 265: 13677-82), GCCR antagonist, RU486 (Taylor, A. I. et al., 2009. Horm. Metab. Res. 41: 899-904), a herbal formula of Polygonati Rhizoma, Rehmanniae Radix, Salviae miltiorrhizae Radix, Puerariae Radix, Schizandrae Fructus, Glycyrrhizae Radix (Kim, J. O. et al., 2009. Biol. Pharm. Bull. 32: 421-6), wheat albumin (Murayama, Y. et al., 2009. J. Agric. Food Chem. 57: 1606-11), n-3 fatty acids (Neschen, S. et al., 2007. Diabetes. 56: 1034-41), dehydroepiandrosterone (Yamashita, R. et al., 2005. Endocr. J. 52: 727-33), S-15261 (Cauzac, M. et al., 2005. Bioechem. Pharmacol. 70: 527-34), adiponectin (Shklyaev, S. et al., 2003. Proc. Natl. Acad. Sci. USA. 100: 14217-22), LXR agonist, T0901317 (Cao, G. et al., 2003. J. Biol. Chem. 278: 1131-6), a combination of fenofibrate and T090317 (Srivastava, R. A. 2009. Eur. J. Pharmacol. 607: 258-63), interferon-gamma (Khazen, W. et al., 2007. Endocrinology. 148: 4007-14), 11beta-hydroxysteroid dehydrogenase type 1 (Berthiaumie, M. et al., 2007. Endocrinology. 148: 2391-7), Salicornia herbacea L (Park, S. H. et al., 2006. Arch. Pharm. Res. 29: 256-64), Ritonavir (Goetzman, E. S. et al., 2003. AIDS Res. Hum. Retroviruses. 19: 1141-50), the synthetic LXR agonist GW3965 (Laffitte, B. A. et al., 2003. Proc. Natl. Acad. Sci. USA. 100: 5419-24), glucocorticoids (Olswang, Y. et al., 2003. J. Biol. Chem. 278: 12929-36), leptin (Burcelin, R. et al., 1999. Diabetes. 48: 1264-9), molybdate (Reul, B. A. et al., 1997. J. Endocrinol. 155: 55-64), dietary n-3 polyunsaturated fatty acids (Raclot, T. et al., 1997. J. Lipid Res. 38: 1963-72), 2,3,7,8-tetrachlorodibenzo-p-dioxin (Viluksela, M. et al., 1995. 135: 308-15), dexamethazone (Franckhauser, S. et al., 1995. Biochem. J. 305: 65-71), tungstate (Munoz, M. C. et al., 2001. Diabetes. 50: 131-8), siRNA against PEPCK (Inoue, Y. et al., 2008. J. Control Release. 126: 59-66), antisense oligonucleotides against FoxO1 (Samuel, V. T. et al., 2006. Diabetes. 55: 2042-50), antisense oligonucleotides against Sirt1 (Erion, D. M. et al., 2009. Proc. Natl. Acad. Sci. USA 106: 11288-93), adenovirus-transduced RNAi against PEPCK-C (Gomez-Valadez, A. G. et al., 2008. Diabetes. 2199-210), and antisense oligonucleotides against Scd1 (Gutierrez-Juarez, R. et al, 2006. J. Clin. Invest. 116: 1686-95).

Previous inhibitor studies on inhibition of PEPCK describe outcomes, such as inhibition of hyperglycemia, hyperlipidemia and hepatic gluconeogenesis, decrease in body weight, increase in insulin sensitivity and increased glucose tolerance. However, none of the inhibitors enumerated above are specific for PEPCK-M and may therefore produce undesirable side-effects.

Antisense inhibition of PEPCK-M provides a unique advantage over traditional small molecule inhibitors in that antisense inhibitors do not rely on competitive binding of the compound to the protein and inhibit activity directly by reducing the expression of PEPCK-M. A representative United States patent that teaches PEPCK-M antisense inhibitors includes U.S. Pat. No. 6,030,837, of which is herein incorporated by reference in its entirety. Furthermore, none of the previously described disclosures describe a specific mechanism of antisense inhibition of PEPCK-M for the treatment of metabolic diseases. Antisense technology is emerging as an effective means for reducing the expression of certain gene products and may therefore prove to be uniquely useful in a number of therapeutic, diagnostic, and research applications for the modulation of PEPCK-M.

There is a currently a lack of acceptable options for treating metabolic disorders. It is therefore an object herein to provide compounds and methods for the treatment of such diseases and disorder.

To date, a specific inhibitor of PEPCK-M has not been identified. It is therefore an object herein to provide compounds and methods for the treatment of such diseases and disorders. This invention relates to the discovery of novel, highly potent inhibitors of PEPCK-M gene expression.

All documents, or portions of documents, cited in this application, including, but not limited to, patents, patent applications, articles, books, and treatises, are hereby expressly incorporated-by-reference for the portions of the document discussed herein, as well as in their entirety.

### SUMMARY OF THE INVENTION

Provided herein are antisense compounds useful for modulating gene expression and associated pathways via antisense mechanisms of action such as RNaseH, RNAi and dsRNA enzymes, as well as other antisense mechanisms based on target degradation or target occupancy.

Provided herein are methods, compounds, and compositions for inhibiting or reducing expression of PEPCK-M and thereby treating, preventing, delaying, decreasing or ameliorating a PEPCK-M related disease, condition or a symptom thereof. In certain embodiments, the PEPCK-M related disease or condition is metabolic disease. In certain embodiments, the PEPCK-M related disease or condition is metabolic disease, including but not limited to diabetes, obesity, metabolic syndrome, diabetic dyslipidemia, or hypertriglyceridemia,

In certain embodiments, the compounds or compositions for the use in the methods provided herein comprise a modified oligonucleotide 10 to 30 linked nucleosides in length targeted to PEPCK-M. The PEPCK-M target can have a sequence selected from any one of SEQ ID NOs: 1-3. The modified oligonucleotide targeting PEPCK-M can have a nucleobase sequence comprising at least 8 contiguous nucleobases complementary to an equal length portion of SEQ ID NOs: 1-3. The modified oligonucleotide can have a nucleobase sequence comprising at least 8, 9, 10, 11, 12, 13, 14, 15, 16, 17, 18, 19, 20, 21, 22, 23, 24, 25, 26, 27, 28, 29 or 30 contiguous nucleobases. The contiguous nucleobase portion of the modified oligonucleotide can be complementary to an equal length portion of a PEPCK-M region selected from any one of SEQ ID NOs: 1-3.

In certain embodiments, the modified oligonucleotide comprises: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar. In certain embodiments, the modified oligonucleotide consists of 20 linked nucleosides, the gap segment consisting of ten linked deoxynucleosides, the 5′ wing segment consisting of five linked nucleosides, the 3′ wing segment consisting of five linked nucleosides, each nucleoside of each wing segment comprises a 2′-O-methoxyethyl sugar, each internucleoside linkage is a phosphorothioate linkage and each cytosine is a 5-methylcytosine.

Certain embodiments provide a method of reducing PEPCK-M expression or activity in an animal comprising administering to the animal a compound comprising the modified oligonucleotide targeting PEPCK-M described herein.

Certain embodiments provide a method of increasing insulin sensitivity or hepatic insulin sensitivity in an animal comprising administering to the animal a compound comprising the modified oligonucleotide targeting PEPCK-M described herein.

Certain embodiments provide a method of reducing insulin, insulin resistance, triglyceride levels, adipose tissue size or weight, body fat, or glucose levels in an animal comprising administering to the animal a compound comprising the modified oligonucleotide targeted to PEPCK-M described herein.

Certain embodiments provide a method of increasing insulin sensitivity or hepatic insulin sensitivity without increasing hypoglycemia in an animal comprising administering to the animal a compound comprising the modified oligonucleotide targeting PEPCK-M described herein.

Certain embodiments provide a method of reducing insulin, insulin resistance, triglyceride levels, adipose tissue size or weight, body fat, or glucose levels without increasing hypoglycemia in an animal comprising administering to the animal a compound comprising the modified oligonucleotide targeted to PEPCK-M described herein. A reduction in body fat can be a reduction in adipose tissue mass, adipocyte size or adipocyte accumulation or a combination thereof.

Certain embodiments provide a method of ameliorating metabolic disease in an animal comprising administering to the animal a compound comprising a modified oligonucleotide targeted to PEPCK-M described herein.

Certain embodiments provide a method of ameliorating metabolic disease in an animal comprising administering to the animal a compound comprising a modified oligonucleotide targeted to PEPCK-M described herein wherein the metabolic disease is diabetes, obesity, metabolic syndrome, diabetic dyslipidemia, or hypertriglyceridemia.

Certain embodiments provide a method for treating an animal with metabolic disease comprising: 1) identifying the animal with metabolic disease, and 2) administering to the animal a therapeutically effective amount of a compound comprising a modified oligonucleotide consisting of 20 linked nucleosides and having a nucleobase sequence at least 90% complementary to SEQ ID NOS: 1-3 as measured over the entirety of said modified oligonucleotide, thereby treating the animal with metabolic disease. In certain embodiments, the therapeutically effective amount of the compound administered to the animal reduces metabolic disease in the animal.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### DEFINITIONS

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques can be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification of the 2′ position of a furosyl ring. A 2′-O-methoxyethyl modified sugar is a modified sugar.

“2′-O-methoxyethyl nucleotide” means a nucleotide comprising a 2′-O-methoxyethyl modified sugar moiety.

“3′ target site” refers to the nucleotide of a target nucleic acid which is complementary to the 3′-most nucleotide of a particular antisense compound.

“5′ target site” refers to the nucleotide of a target nucleic acid which is complementary to the 5′-most nucleotide of a particular antisense compound.

“5-methylcytosine” means a cytosine modified with a methyl group attached to the 5′ position. A 5-methylcytosine is a modified nucleobase.

“Active pharmaceutical agent” means the substance or substances in a pharmaceutical composition that provide a therapeutic benefit when administered to an individual. For example, in certain embodiments an antisense oligonucleotide targeted to PEPCK-M is an active pharmaceutical agent.

“Active target region” or “target region” means a region to which one or more active antisense compounds is targeted. “Active antisense compounds” means antisense compounds that reduce target nucleic acid levels or protein levels.

“Adipogenesis” means the development of fat cells from preadipocytes. “Lipogenesis” means the production or formation of fat, either fatty degeneration or fatty infiltration.

“Adiposity” or “Obesity” refers to the state of being obese or an excessively high amount of body fat or adipose tissue in relation to lean body mass. The amount of body fat includes concern for both the distribution of fat throughout the body and the size and mass of the adipose tissue deposits. Body fat distribution can be estimated by skin-fold measures, waist-to-hip circumference ratios, or techniques such as ultrasound, computed tomography, or magnetic resonance imaging. According to the Center for Disease Control and Prevention, individuals with a body mass index (BMI) of 30 or more are considered obese. The term “Obesity” as used herein includes conditions where there is an increase in body fat beyond the physical requirement as a result of excess accumulation of adipose tissue in the body. The term “obesity” includes, but is not limited to, the following conditions: adult-onset obesity; alimentary obesity; endogenous or inflammatory obesity; endocrine obesity; familial obesity; hyperinsulinar obesity; hyperplastic-hypertrophic obesity; hypogonadal obesity; hypothyroid obesity; lifelong obesity; morbid obesity and exogenous obesity.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Administering” means providing an agent to an animal, and includes, but is not limited to, administering by a medical professional and self-administering.

“Agent” means an active substance that can provide a therapeutic benefit when administered to an animal. “First Agent” means a therapeutic compound of the invention. For example, a first agent can be an antisense oligonucleotide targeting PEPCK-M. “Second agent” means a second therapeutic compound of the invention (e.g. a second antisense oligonucleotide targeting PEPCK-M) and/or a non-PEPCK-M therapeutic compound.

“Amelioration” refers to a lessening of at least one indicator, sign, or symptom of an associated disease, disorder, or condition. The severity of indicators can be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid.

“Bicyclic sugar” means a furosyl ring modified by the bridging of two non-geminal ring atoms. A bicyclic sugar is a modified sugar.

“Bicyclic nucleic acid” or “BNA” refers to a nucleoside or nucleotide wherein the furanose portion of the nucleoside or nucleotide includes a bridge connecting two carbon atoms on the furanose ring, thereby forming a bicyclic ring system.

“Cap structure” or “terminal cap moiety” means chemical modifications, which have been incorporated at either terminus of an antisense compound.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions.

“Co-administration” means administration of two or more agents to an individual. The two or more agents can be in a single pharmaceutical composition, or can be in separate pharmaceutical compositions. Each of the two or more agents can be administered through the same or different routes of administration. Co-administration encompasses parallel or sequential administration.

“Cholesterol” is a sterol molecule found in the cell membranes of all animal tissues. Cholesterol must be transported in an animal's blood plasma by lipoproteins including very low density lipoprotein (VLDL), intermediate density lipoprotein (IDL), low density lipoprotein (LDL), and high density lipoprotein (HDL). “Plasma cholesterol” refers to the sum of all lipoproteins (VDL, IDL, LDL, HDL) esterified and/or non-estrified cholesterol present in the plasma or serum.

“Cholesterol absorption inhibitor” means an agent that inhibits the absorption of exogenous cholesterol obtained from diet.

“Complementarity” means the capacity for pairing between nucleobases of a first nucleic acid and a second nucleic acid.

“Contiguous nucleobases” means nucleobases immediately adjacent to each other.

“Deoxyribonucleotide” means a nucleotide having a hydrogen at the 2′ position of the sugar portion of the nucleotide. Deoxyribonucleotides may be modified with any of a variety of substituents.

“Diabetes mellitus” or “diabetes” is a syndrome characterized by disordered metabolism and abnormally high blood sugar (hyperglycemia) resulting from insufficient levels of insulin or reduced insulin sensitivity. The characteristic symptoms are excessive urine production (polyuria) due to high blood glucose levels, excessive thirst and increased fluid intake (polydipsia) attempting to compensate for increased urination, blurred vision due to high blood glucose effects on the eye's optics, unexplained weight loss, and lethargy.

“Diabetic dyslipidemia” or “type 2 diabetes with dyslipidemia” means a condition characterized by Type 2 diabetes, reduced HDL-C, elevated triglycerides, and elevated small, dense LDL particles.

“Diluent” means an ingredient in a composition that lacks pharmacological activity, but is pharmaceutically necessary or desirable. For example, the diluent in an injected composition can be a liquid, e.g. saline solution.

“Dyslipidemia” refers to a disorder of lipid and/or lipoprotein metabolism, including lipid and/or lipoprotein overproduction or deficiency. Dyslipidemias may be manifested by elevation of lipids such as cholesterol and triglycerides as well as lipoproteins such as low-density lipoprotein (LDL) cholesterol.

“Dosage unit” means a form in which a pharmaceutical agent is provided, e.g. pill, tablet, or other dosage unit known in the art. In certain embodiments, a dosage unit is a vial containing lyophilized antisense oligonucleotide. In certain embodiments, a dosage unit is a vial containing reconstituted antisense oligonucleotide.

“Dose” means a specified quantity of a pharmaceutical agent provided in a single administration, or in a specified time period. In certain embodiments, a dose can be administered in one, two, or more boluses, tablets, or injections. For example, in certain embodiments where subcutaneous administration is desired, the desired dose requires a volume not easily accommodated by a single injection, therefore, two or more injections can be used to achieve the desired dose. In certain embodiments, the pharmaceutical agent is administered by infusion over an extended period of time or continuously. Doses can be stated as the amount of pharmaceutical agent per hour, day, week, or month.

“Effective amount” or “therapeutically effective amount” means the amount of active pharmaceutical agent sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

“Fully complementary” or “100% complementary” means each nucleobase of a nucleobase sequence of a first nucleic acid has a complementary nucleobase in a second nucleobase sequence of a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region can be referred to as a “gap segment” and the external regions can be referred to as “wing segments.”

“Gap-widened” means a chimeric antisense compound having a gap segment of 12 or more contiguous 2′-deoxyribonucleosides positioned between and immediately adjacent to 5′ and 3′ wing segments having from one to six nucleosides.

“Glucose” is a monosaccharide used by cells as a source of energy and inflammatory intermediate. “Plasma glucose” refers to glucose present in the plasma.

“HMG-CoA reductase inhibitor” means an agent that acts through the inhibition of the enzyme HMG-CoA reductase, such as atorvastatin, rosuvastatin, fluvastatin, lovastatin, pravastatin, and simvastatin.

“Hybridization” means the annealing of complementary nucleic acid molecules. In certain embodiments, complementary nucleic acid molecules include an antisense compound and a target nucleic acid.

“Hyperlipidemia” or “hyperlipemia” is a condition characterized by elevated serum lipids or circulating (plasma) lipids. This condition manifests an abnormally high concentration of fats. The lipid fractions in the circulating blood are cholesterol, low density lipoproteins, very low density lipoproteins and triglycerides.

“Hypertriglyceridemia” means a condition characterized by elevated triglyceride levels.

“Identifying” or “selecting an animal with metabolic” means identifying or selecting a subject having been diagnosed with a metabolic disease, or a metabolic disorder; or, identifying or selecting a subject having any symptom of a metabolic disease, including, but not limited to, metabolic syndrome, hyperglycemia, hypertriglyceridemia, hypertension increased insulin resistance, decreased insulin sensitivity, above normal body weight, and/or above normal body fat or any combination thereof. Such identification may be accomplished by any method, including but not limited to, standard clinical tests or assessments, such as measuring serum or circulating (plasma) blood-glucose, measuring serum or circulating (plasma) triglycerides, measuring blood-pressure, measuring body fat, measuring body weight, and the like.

“Immediately adjacent” means there are no intervening elements between the immediately adjacent elements.

“Individual” or “subject” or “animal” means a human or non-human animal selected for treatment or therapy.

“Inhibiting the expression or activity” refers to a reduction or blockade of the expression or activity of a RNA or protein and does not necessarily indicate a total elimination of expression or activity.

“Insulin resistance” is defined as the condition in which normal amounts of insulin are inadequate to produce a normal insulin response from fat, muscle and liver cells. Insulin resistance in fat cells results in hydrolysis of stored triglycerides, which elevates free fatty acids in the blood plasma. Insulin resistance in muscle reduces glucose uptake whereas insulin resistance in liver reduces glucose storage, with both effects serving to elevate blood glucose. High plasma levels of insulin and glucose due to insulin resistance often leads to metabolic syndrome and type 2 diabetes.

“Insulin sensitivity” is a measure of how effectively an individual processes glucose. An individual having high insulin sensitivity effectively processes glucose whereas an individual with low insulin sensitivity does not effectively process glucose.

“Internucleoside linkage” refers to the chemical bond between nucleosides.

“Intravenous administration” means administration into a vein.

“Linked nucleosides” means adjacent nucleosides which are bonded together.

“Lipid-lowering therapy” or “lipid lowering agent” means a therapeutic regimen provided to a subject to reduce one or more lipids in a subject. In certain embodiments, a lipid-lowering therapy is provided to reduce one or more of ApoB, total cholesterol, LDL-C, VLDL-C, IDL-C, non-HDL-C, triglycerides, small dense LDL particles, and Lp(a) in a subject. Examples of lipid-lowering therapy include statins, fibrates, and MTP inhibitors.

“Major risk factors” refers to factors that contribute to a high risk for a particular disease or condition. In certain embodiments, major risk factors for coronary heart disease include, without limitation, cigarette smoking, hypertension, low HDL-C, family history of coronary heart disease, age, and other factors disclosed herein.

“Metabolic disease” or “metabolic disorder” refers to a condition characterized by an alteration or disturbance in metabolic function. “Metabolic” and “metabolism” are terms well known in the art and generally include the whole range of biochemical processes that occur within a living organism. Metabolic diseases or disorders include, but are not limited to, obesity, diabetes, hyperglycemia, prediabetes, non-alcoholic fatty liver disease (NAFLD), metabolic syndrome, insulin resistance, diabetic dyslipidemia, or hypertriglyceridemia or a combination thereof.

“Metabolic syndrome” means a condition characterized by a clustering of lipid and non-lipid cardiovascular risk factors of metabolic origin. In certain embodiments, metabolic syndrome is identified by the presence of any 3 of the following factors: waist circumference of greater than 102 cm in men or greater than 88 cm in women; serum triglyceride of at least 150 mg/dL; HDL-C less than 40 mg/dL in men or less than 50 mg/dL in women; blood pressure of at least 130/85 mmHg; and fasting glucose of at least 110 mg/dL. These determinants can be readily measured in clinical practice (JAMA, 2001, 285: 2486-2497).

“Mismatch” or “non-complementary nucleobase” refers to the case when a nucleobase of a first nucleic acid is not capable of pairing with the corresponding nucleobase of a second or target nucleic acid.

“Mixed dyslipidemia” means a condition characterized by elevated cholesterol and elevated triglycerides.

“Modified internucleoside linkage” refers to a substitution or any change from a naturally occurring internucleoside bond (i.e. a phosphodiester internucleoside bond).

“Modified nucleobase” refers to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleoside” means a nucleoside having, independently, a modified sugar moiety or modified nucleobase.

“Modified nucleotide” means a nucleotide having, independently, a modified sugar moiety, modified internucleoside linkage, or modified nucleobase. A “modified nucleoside” means a nucleoside having, independently, a modified sugar moiety or modified nucleobase.

“Modified oligonucleotide” means an oligonucleotide comprising at least one modified nucleotide.

“Modified sugar” refers to a substitution or change from a natural sugar.

“Motif” means the pattern of chemically distinct regions in an antisense compound.

“MTP inhibitor” means an agent inhibits the enzyme, microsomal triglyceride transfer protein.

“Naturally occurring internucleoside linkage” means a 3′ to 5′ phosphodiester linkage.

“Natural sugar moiety” means a sugar found in DNA (2′-H) or RNA (2′-OH).

“Non-alcoholic fatty liver disease” or “NAFLD” means a condition characterized by fatty inflammation of the liver that is not due to excessive alcohol use (for example, alcohol consumption of over 20 g/day). In certain embodiments, NAFLD is related to insulin resistance and the metabolic syndrome. NAFLD encompasses a disease spectrum ranging from simple triglyceride accumulation in hepatocytes (hepatic steatosis) to hepatic steatosis with inflammation (steatohepatitis), fibrosis, and cirrhosis.

“Nonalcoholic steatohepatitis” (NASH) occurs from progression of NAFLD beyond deposition of triglycerides. A “second hit” capable of inducing necrosis, inflammation, and fibrosis is required for development of NASH. Candidates for the second-hit can be grouped into broad categories: factors causing an increase in oxidative stress and factors promoting expression of proinflammatory cytokines

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA). A nucleic acid can also comprise a combination of these elements in a single molecule.

“Nucleobase” means a heterocyclic moiety capable of pairing with a base of another nucleic acid.

“Nucleobase sequence” means the order of contiguous nucleobases independent of any sugar, linkage, or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics e.g. non furanose sugar units.

“Nucleotide” means a nucleoside having a phosphate group covalently linked to the sugar portion of the nucleoside.

“Nucleotide mimetic” includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage).

“Oligomeric compound” or “oligomer” refers to a polymeric structure comprising two or more sub-structures and capable of hybridizing to a region of a nucleic acid molecule. In certain embodiments, oligomeric compounds are oligonucleosides. In certain embodiments, oligomeric compounds are oligonucleotides. In certain embodiments, oligomeric compounds are antisense compounds. In certain embodiments, oligomeric compounds are antisense oligonucleotides. In certain embodiments, oligomeric compounds are chimeric oligonucleotides.

“Oligonucleotide” means a polymer of linked nucleosides each of which can be modified or unmodified, independent one from another.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration. Administration can be continuous, or chronic, or short or intermittent.

“Phosphoenolpyruvate carboxykinase-2” or “PEPCK-M” (also known as PCK2; PEPCK-2; PEPCK-M; phosphoenolpyruvate carboxykinase-2; phosphoenolpyruvate carboxykinase-mitochondrial) means any nucleic acid or protein of PEPCK-M.

“PEPCK-M expression” means the level of mRNA transcribed from the gene encoding PEPCK-M or the level of protein translated from the mRNA. PEPCK-M expression can be determined by art known methods such as a Northern or Western blot.

“PEPCK-M nucleic acid” means any nucleic acid encoding PEPCK-M. For example, in certain embodiments, a PEPCK-M nucleic acid includes a DNA sequence encoding PEPCK-M, a RNA sequence transcribed from DNA encoding PEPCK-M (including genomic DNA comprising introns and exons), and a mRNA sequence encoding PEPCK-M. “PEPCK-M mRNA” means a mRNA encoding a PEPCK-M protein.

“Peptide” means a molecule formed by linking at least two amino acids by amide bonds. Peptide refers to polypeptides and proteins.

“Pharmaceutical agent” means a substance that provides a therapeutic benefit when administered to an individual. For example, in certain embodiments, an antisense oligonucleotide targeted to PEPCK-M is pharmaceutical agent.

“Pharmaceutical composition” means a mixture of substances suitable for administering to an individual. For example, a pharmaceutical composition can comprise one or more active agents and a sterile aqueous solution.

“Pharmaceutically acceptable carrier” means a medium or diluent that does not interfere with the structure of the oligonucleotide. Certain, of such carries enable pharmaceutical compositions to be formulated as, for example, tablets, pills, dragees, capsules, liquids, gels, syrups, slurries, suspension and lozenges for the oral ingestion by a subject. For example, a pharmaceutically acceptable carrier can be a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” refers to delaying or forestalling the onset or development of a disease, disorder, or condition for a period of time from minutes to indefinitely. Prevent also means reducing risk of developing a disease, disorder, or condition.

“Prodrug” means a therapeutic agent that is prepared in an inactive form that is converted to an active form within the body or cells thereof by the action of endogenous enzymes or other chemicals or conditions.

“Side effects” means physiological responses attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum can indicate liver toxicity or liver function abnormality. For example, increased bilirubin can indicate liver toxicity or liver function abnormality.

“Single-stranded oligonucleotide” means an oligonucleotide which is not hybridized to a complementary strand.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e. under physiological conditions in the case of in vivo assays and therapeutic treatments.

“Statin” means an agent that inhibits the activity of HMG-CoA reductase.

“Subcutaneous administration” means administration just below the skin.

“Targeting” or “targeted” means the process of design and selection of an antisense compound that will specifically hybridize to a target nucleic acid and induce a desired effect.

“Target nucleic acid,” “target RNA,” and “target RNA transcript” all refer to a nucleic acid capable of being targeted by antisense compounds.

“Target segment” means the sequence of nucleotides of a target nucleic acid to which an antisense compound is targeted. “5′ target site” refers to the 5′-most nucleotide of a target segment.

“3′ target site” refers to the 3′-most nucleotide of a target segment.

“Therapeutically effective amount” means an amount of an agent that provides a therapeutic benefit to an individual.

“Therapeutic lifestyle change” means dietary and lifestyle changes intended to lower fat/adipose tissue mass and/or cholesterol. Such change can reduce the risk of developing heart disease, and may includes recommendations for dietary intake of total daily calories, total fat, saturated fat, polyunsaturated fat, monounsaturated fat, carbohydrate, protein, cholesterol, insoluble fiber, as well as recommendations for physical activity.

“Triglyceride” or “TG” means a lipid or neutral fat consisting of glycerol combined with three fatty acid molecules.

“Type 2 diabetes,” (also known as “type 2 diabetes mellitus” or “diabetes mellitus, type 2”, and formerly called “diabetes mellitus type 2”, “non-insulin-dependent diabetes (NIDDM)”, “obesity related diabetes”, or “adult-onset diabetes”) is a metabolic disorder that is primarily characterized by insulin resistance, relative insulin deficiency, and hyperglycemia.

“Treat” refers to administering a pharmaceutical composition to an animal to effect an alteration or improvement of a disease, disorder, or condition.

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

### Certain Embodiments

In certain embodiments, the compounds or compositions for the use in the methods provided herein comprise a modified oligonucleotide 10 to 30 linked nucleosides in length targeted to PEPCK-M. The PEPCK-M target can have a sequence selected from any one of SEQ ID NOs: 1-3.

In certain embodiments, the compounds or compositions for the use in the methods provided herein comprise a modified oligonucleotide consisting of 10 to 30 nucleosides having a nucleobase sequence comprising at least 8 contiguous nucleobases complementary to an equal length portion of SEQ ID NOs: 1-3.

In certain embodiments, the compounds or compositions for the use in the methods provided herein comprise a modified oligonucleotide consisting of 10 to 30 linked nucleosides and having a nucleobase sequence comprising at least 8, 9, 10, 11, 12, 13, 14, 15, 16, 17, 18, 19, 20, 21, 22, 23, 24, 25, 26, 27, 28, 29 or 30 contiguous nucleobases complementary to an equal length portion of SEQ ID NOs: 1-3.

In certain embodiments, the compounds or compositions for the use in the methods provided herein can consist of 10 to 30 linked nucleosides and have a nucleobase sequence comprising at least 8, 9, 10, 11, 12, 13, 14, 15, 16, 17, 18, 19, or 20 contiguous nucleobases of any of SEQ ID NO: 9-48.

In certain embodiments, the following antisense compounds or oligonucleotides for the use in the methods target a region of a PEPCK-M nucleic acid and effect at least a 60% inhibition of a PEPCK-M mRNA: ISIS ID NOs: 104154, 104169, 104174, 104176, 104178, 104180, 104182, 104183, 104187, 104189, 104192, 104196, 104198, 104201, 104203, 104205, and 104207.

In certain embodiments, the following antisense compounds or oligonucleotides for the use in the methods target a region of a PEPCK-M nucleic acid and effect at least a 65% inhibition of a PEPCK-M mRNA: ISIS ID NOs: 104154, 104169, 104174, 104176, 104178, 104180, 104182, 104183, 104192, 104196, 104198, 104201, 104203, and 104205.

In certain embodiments, the following antisense compounds or oligonucleotides for the use in the methods target a region of a PEPCK-M nucleic acid and effect at least a 70% inhibition of a PEPCK-M mRNA: ISIS ID NOs: 104169, 104174, 104176, 104180, 104182, 104183, 104192, 104198, 104201, 104203, and 104205.

In certain embodiments, the following antisense compounds or oligonucleotides for the use in the methods target a region of a PEPCK-M nucleic acid and effect at least a 75% inhibition of a PEPCK-M mRNA: ISIS ID NOs: 104169, 104174, 104176, 104180, 104183, 104192, 104201, and 104203.

In certain embodiments, the following antisense compounds or oligonucleotides for the use in the methods target a region of a PEPCK-M nucleic acid and effect at least a 80% inhibition of a PEPCK-M mRNA: ISIS ID NOs: 104176, 104180, 104192, and 104201.

In certain embodiments, the following antisense compounds or oligonucleotides for the use in the methods target a region of a PEPCK-M nucleic acid and effect at least a 85% inhibition of a PEPCK-M mRNA: ISIS ID NO: 104176

In certain embodiments, antisense compounds or oligonucleotides for the use in the methods target a region of a PEPCK-M nucleic acid. In certain embodiments, an antisense compound or oligonucleotide targeted to a PEPCK-M nucleic acid can target the following nucleotide regions of SEQ ID NO: 1: 1537-1556, 84-103, 308-327, 443-591, 443-462, 572-591, 696-715, 805-871, 805-824, 852-871, 1028-1047, 1142-1161, 1343-1362, 1646-1665, 1770-1789, 1939-1958, 2036-2113, 2036-2055, 2094-2113, and 2170-2189.

In certain embodiment, compounds or oligonucleotides for the use in the methods targeted to a region of a PEPCK-M nucleic acid can have a contiguous nucleobase portion that is complementary to an equal length nucleobase portion of the region. For example, the portion can be at least an 8, 9, 10, 11, 12, 13, 14, 15, 16, 17, 18, 19 or 20 contiguous nucleobases portion complementary to an equal length portion of SEQ ID NO: 1 region: 1537-1556, 84-103, 308-327, 443-591, 443-462, 572-591, 696-715, 805-871, 805-824, 852-871, 1028-1047, 1142-1161, 1343-1362, 1646-1665, 1770-1789, 1939-1958, 2036-2113, 2036-2055, 2094-2113, and 2170-2189.

In certain embodiments, the following nucleotide regions of SEQ ID NO: 1, when targeted by antisense compounds or oligonucleotides, display at least 60% inhibition of PEPCK-M: 1537-1556, 84-103, 308-327, 443-591, 443-462, 572-591, 696-715, 805-871, 805-824, 852-871, 1028-1047, 1142-1161, 1343-1362, 1646-1665, 1770-1789, 1939-1958, 2036-2113, 2036-2055, 2094-2113, and 2170-2189.

In certain embodiments, the following nucleotide regions of SEQ ID NO: 1, when targeted by antisense compounds or oligonucleotides, display at least 65% inhibition of PEPCK-M: 1537-1556, 84-103, 308-327, 443-591, 443-462, 572-591, 696-715, 805-871, 805-824, 852-871, 1343-1362, 1646-1665, 1770-1789, 1939-1958, 2036-2113, 2036-2055, and 2094-2113.

In certain embodiments, the following nucleotide regions of SEQ ID NO: 1, when targeted by antisense compounds or oligonucleotides, display at least 70% inhibition of PEPCK-M: 84-103, 308-327, 443-462, 696-715, 805-871, 805-824, 852-871, 1343-1362, 1770-1789, 1939-1958, 2036-2113, 2036-2055, and 2094-2113.

In certain embodiments, the following nucleotide regions of SEQ ID NO: 1, when targeted by antisense compounds or oligonucleotides, display at least 75% inhibition of PEPCK-M: 84-103, 308-327, 443-462, 696-715, 852-871, 1343-1362, 1939-1958, and 2036-2055.

In certain embodiments, the following nucleotide regions of SEQ ID NO: 1, when targeted by antisense compounds or oligonucleotides, display at least 80% inhibition of PEPCK-M: 443-462, 696-715, 1343-1362, and 1939-1958.

In certain embodiments, antisense compounds or oligonucleotides target a region of a PEPCK-M nucleic acid. In certain embodiments, an antisense compound or oligonucleotide targeted to a PEPCK-M nucleic acid can target the following nucleotide regions of SEQ ID NO: 2: 12242-12261, 3407-3426, 6088-6107, 7288-7307, 7417-7436, 7628-7647, 8107-8126, 8154-8173, 8651-8670, 9240-9259, 12605-12624, 12729-12748, 12898-12917, 13053-13072, and 13129-13148.

In certain embodiment, compounds or oligonucleotides for the use in the methods targeted to a region of a PEPCK-M nucleic acid can have a contiguous nucleobase portion that is complementary to an equal length nucleobase portion of the region. For example, the portion can be at least an 8, 9, 10, 11, 12, 13, 14, 15, 16, 17, 18, 19 or 20 contiguous nucleobases portion complementary to an equal length portion of SEQ ID NO: 2 region: 12242-12261, 3407-3426, 6088-6107, 7288-7307, 7417-7436, 7628-7647, 8107-8126, 8154-8173, 8651-8670, 9240-9259, 12605-12624, 12729-12748, 12898-12917, 13053-13072, and 13129-13148.

In certain embodiments, the following nucleotide regions of SEQ ID NO: 2, when targeted by antisense compounds or oligonucleotides, display at least 60% inhibition of PEPCK-M: 12242-12261, 3407-3426, 6088-6107, 7288-7307, 7417-7436, 7628-7647, 8107-8126, 8154-8173, 8651-8670, 9240-9259, 12605-12624, 12729-12748, 12898-12917, 13053-13072, and 13129-13148.

In certain embodiments, the following nucleotide regions of SEQ ID NO: 2, when targeted by antisense compounds or oligonucleotides, display at least 65% inhibition of PEPCK-M: 12242-12261, 3407-3426, 6088-6107, 7288-7307, 7417-7436, 7628-7647, 8107-8126, 8154-8173, 9240-9259, 12605-12624, 12729-12748, 12898-12917, and 13053-13072.

In certain embodiments, the following nucleotide regions of SEQ ID NO: 2, when targeted by antisense compounds or oligonucleotides, display at least 70% inhibition of PEPCK-M: 3407-3426, 6088-6107, 7288-7307, 7628-7647, 8107-8126, 8154-8173, 9240-9259, 12729-12748, 12898-12917, and 13053-13072.

In certain embodiments, the following nucleotide regions of SEQ ID NO: 2, when targeted by antisense compounds or oligonucleotides, display at least 75% inhibition of PEPCK-M: 3407-3426, 6088-6107, 7288-7307, 7628-7647, 8154-8173, 9240-9259, and 12898-12917.

In certain embodiments, the following nucleotide regions of SEQ ID NO: 2, when targeted by antisense compounds or oligonucleotides, display at least 80% inhibition of PEPCK-M: 7288-7307, 7628-7647, 8154-8173, 9240-9259, and 12898-12917.

In certain embodiments, antisense compounds or oligonucleotides for the use in the methods target a region of a PEPCK-M nucleic acid. In certain embodiments, an antisense compound or oligonucleotide targeted to a PEPCK-M nucleic acid can target the following nucleotide regions of SEQ ID NO: 3: 1471-1490, 18-37, 242-261, 377-396, 506-525, 630-649, 739-758, 786-805, 962-981, 1076-1095, 1277-1296, 1580-1599, 1704-1723, 1873-1892, 1970-1989, 2027-2046, and 2102-2121.

In certain embodiment, compounds or oligonucleotides for the use in the methods targeted to a region of a PEPCK-M nucleic acid can have a contiguous nucleobase portion that is complementary to an equal length nucleobase portion of the region. For example, the portion can be at least an 8, 9, 10, 11, 12, 13, 14, 15, 16, 17, 18, 19 or 20 contiguous nucleobases portion complementary to an equal length portion of SEQ ID NO: 3 region: 1471-1490, 18-37, 242-261, 377-396, 506-525, 630-649, 739-758, 786-805, 962-981, 1076-1095, 1277-1296, 1580-1599, 1704-1723, 1873-1892, 1970-1989, 2027-2046, and 2102-2121.

In certain embodiments, the following nucleotide regions of SEQ ID NO: 3, when targeted by antisense compounds or oligonucleotides, display at least 60% inhibition of PEPCK-M: 1471-1490, 18-37, 242-261, 377-396, 506-525, 630-649, 739-758, 786-805, 962-981, 1076-1095, 1277-1296, 1580-1599, 1704-1723, 1873-1892, 1970-1989, 2027-2046, and 2102-2121.

In certain embodiments, the following nucleotide regions of SEQ ID NO: 3, when targeted by antisense compounds or oligonucleotides, display at least 65% inhibition of PEPCK-M: 1471-1490, 18-37, 242-261, 377-396, 506-525, 630-649, 739-758, 786-805, 1277-1296, 1580-1599, 1704-1723, 1873-1892, 1970-1989, and 2027-2046.

In certain embodiments, the following nucleotide regions of SEQ ID NO: 3, when targeted by antisense compounds or oligonucleotides, display at least 70% inhibition of PEPCK-M: 18-37, 242-261, 377-396, 630-649, 739-758, 786-805, 1277-1296, 1704-1723, 1873-1892, 1970-1989, and 2027-2046.

In certain embodiments, the following nucleotide regions of SEQ ID NO: 3, when targeted by antisense compounds or oligonucleotides, display at least 75% inhibition of PEPCK-M: 18-37, 242-261, 377-396, 630-649, 786-805, 1277-1296, 1873-1892, and 1970-1989.

In certain embodiments, the following nucleotide regions of SEQ ID NO: 3, when targeted by antisense compounds or oligonucleotides, display at least 80% inhibition of PEPCK-M: 377-396, 630-649, 1277-1296, and 1873-1892.

In certain embodiments, the compounds or compositions for the use in the methods provided herein comprise a salt of the modified oligonucleotide.

In certain embodiments, the compounds or compositions for the use in the methods provided herein further comprise a pharmaceutically acceptable carrier or diluent.

In certain embodiments, the nucleobase sequence of the modified oligonucleotide is at least 70%, 80%, 90%, 95% or 100% complementary to any one of SEQ ID NOs: 1-3 as measured over the entirety of the modified oligonucleotide.

In certain embodiments, the compound for the use in the methods provided herein consists of a single-stranded modified oligonucleotide.

In certain embodiments, the modified oligonucleotide consists of 8, 9, 10, 11, 12, 13, 14, 15, 16, 17, 18, 19, 20, 21, 22, 23, 24, 25, 26, 27, 28, 29 or 30 linked nucleosides. In certain embodiments, the modified oligonucleotide consists of 20 linked nucleosides.

In certain embodiments, at least one internucleoside linkage of said modified oligonucleotide is a modified internucleoside linkage. In certain embodiments, each internucleoside linkage is a phosphorothioate internucleoside linkage.

In certain embodiments, at least one nucleoside of the modified oligonucleotide comprises a modified sugar. In certain embodiments, the modified oligonucleotide comprises at least one tetrahydropyran modified nucleoside wherein a tetrahydropyran ring replaces a furanose ring. In certain embodiments each of the tetrahydropyran modified nucleoside has the structure:

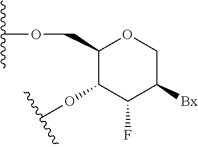

wherein Bx is an optionally protected heterocyclic base moiety. In certain embodiments, at least one modified sugar is a bicyclic sugar. In certain embodiments, at least one modified sugar comprises a 2′-O-methoxyethyl or a 4′-(CH₂)ₙ—O-2′ bridge, wherein n is 1 or 2.

In certain embodiments, at least one nucleoside of said modified oligonucleotide comprises a modified nucleobase. In certain embodiments, the modified nucleobase is a 5-methylcytosine.

In certain embodiments, the modified oligonucleotide comprises: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar. In certain embodiments, the modified oligonucleotide consists of 20 linked nucleosides, the gap segment consisting of ten linked deoxynucleosides, the 5′ wing segment consisting of five linked nucleosides, the 3′ wing segment consisting of five linked nucleosides, each nucleoside of each wing segment comprises a 2′-O-methoxyethyl sugar, each internucleoside linkage is a phosphorothioate linkage and each cytosine is a 5-methylcytosine.

In certain embodiments, the compounds or compositions for the use in the methods provided herein comprise a modified oligonucleotide consists of 20 linked nucleosides having a nucleobase sequence comprising at least 8 contiguous nucleobases complementary to an equal length portion of any of SEQ ID NOs: 1-3, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage is a phosphorothioate linkage and each cytosine residue is a 5-methylcytosine.

Certain embodiments provide methods, compounds, and compositions for inhibiting PEPCK-M expression.

Certain embodiments provide a method of reducing PEPCK-M expression in an animal comprising administering to the animal a compound for the use in the methods provided herein described herein. In certain embodiments, the compound comprises a modified oligonucleotide 10 to 30 linked nucleosides in length targeted to PEPCK-M.

Certain embodiments provide a method of reducing PEPCK-M activity in an animal comprising administering to the animal a compound for the use in the methods provided herein described herein. In certain embodiments, the compound comprises a modified oligonucleotide 10 to 30 linked nucleosides in length targeted to PEPCK-M.

Certain embodiments provide a method of increasing insulin sensitivity or hepatic insulin sensitivity in an animal comprising administering to the animal a compound for the use in the methods provided herein described herein. In certain embodiments, the compound comprises a modified oligonucleotide 10 to 30 linked nucleosides in length targeted to PEPCK-M. In certain embodiments, insulin sensitivity or hepatic insulin sensitivity is increased by at least 5%, 10%, 20%, 30%, 35%, 40%, 45%, 50%, 55%, 60%, 65%, 70%, 75%, 80%, 85%, 90%, 95% or 100%.

Certain embodiments provide a method of increasing insulin sensitivity or hepatic insulin sensitivity without causing hypoglycemia in an animal comprising administering to the animal a compound for the use in the methods provided herein described herein. In certain embodiments, the compound comprises a modified oligonucleotide 10 to 30 linked nucleosides in length targeted to PEPCK-M. In certain embodiments, insulin sensitivity or hepatic insulin sensitivity is increased by at least 5%, 10%, 20%, 30%, 35%, 40%, 45%, 50%, 55%, 60%, 65%, 70%, 75%, 80%, 85%, 90%, 95% or 100%.

Certain embodiments provide a method of reducing body weight, body fat, blood glucose, insulin resistance, triglyceride levels, or insulin levels in an animal comprising administering to the animal a compound for the use in the methods provided herein described herein. In certain embodiments, the compound comprises a modified oligonucleotide 10 to 30 linked nucleosides in length targeted to PEPCK-M. In certain embodiments, body weight, body fat, blood glucose, insulin resistance, triglyceride levels, or insulin levels is decreased by at least 5%, 10%, 20%, 30%, 35%, 40%, 45%, 50%, 55%, 60%, 65%, 70%, 75%, 80%, 85%, 90%, 95% or 100%.

Certain embodiments provide a method of reducing body weight, body fat, blood glucose, insulin resistance, triglyceride levels, or insulin levels without causing hypoglycemia in an animal comprising administering to the animal a compound for the use in the methods provided herein described herein. In certain embodiments, the compound comprises a modified oligonucleotide 10 to 30 linked nucleosides in length targeted to PEPCK-M. In certain embodiments, body weight, body fat, blood glucose, insulin resistance, triglyceride levels, or insulin levels is decreased by at least 5%, 10%, 20%, 30%, 35%, 40%, 45%, 50%, 55%, 60%, 65%, 70%, 75%, 80%, 85%, 90%, 95% or 100%.

Certain embodiments provide a method of a preventing or ameliorating metabolic disease in an animal comprising administering to the animal a compound for the use in the methods provided herein described herein. In certain embodiments, the compound comprises a modified oligonucleotide 10 to 30 linked nucleosides in length targeted to PEPCK-M. In certain embodiments, the metabolic disease is diabetes. In certain embodiments, the metabolic disease is obesity. In certain embodiments, the metabolic disease is metabolic syndrome. In certain embodiments, the metabolic disease is diabetic dyslipidemia. In certain embodiments, the metabolic disease is hypertriglyceridemia.

Certain embodiments provide a method for treating an animal with metabolic disease comprising: a) identifying said animal with metabolic disease, and b) administering to said animal a therapeutically effective amount of a compound comprising a modified oligonucleotide consisting of 20 linked nucleosides and having a nucleobase sequence at least 90% complementary to any of SEQ ID NOs: 1-3 as measured over the entirety of said modified oligonucleotide.

Certain embodiments provide a method for treating an animal with diabetes, obesity, metabolic syndrome, diabetic dyslipidemia, or hypertriglyceridemia comprising a) identifying said animal with diabetes, obesity, metabolic syndrome, diabetic dyslipidemia, or hypertriglyceridemia, and b) administering to said animal a therapeutically effective amount of an antisense oligonucleotide consisting of 20 linked nucleosides and having a nucleobase sequence at least 90% complementary to SEQ ID NOs: 1-3 as measured over the entirety of said antisense oligonucleotide.

Certain embodiments provide a method for treating an animal with diabetes, obesity, metabolic syndrome, diabetic dyslipidemia, or hypertriglyceridemia comprising a) administering to said animal a therapeutically effective amount of an antisense oligonucleotide consisting of 20 linked nucleosides, and b) having a nucleobase sequence comprising at least 8 contiguous nucleobases of a nucleobase sequence selected from any one of SEQ ID NOs: 9-48 and c) comprising a gap segment consisting of ten linked deoxynucleosides; and a 5′ wing segment consisting of five linked nucleosides; and a 3′ wing segment consisting of five linked nucleosides; wherein the gap segment is positioned between the 5′ wing segment and the 3′ wing segment, and wherein each nucleoside of each wing segment comprises a 2′-O-methoxyethyl sugar, and wherein each internucleoside linkage is a phosphorothioate linkage, and wherein each cytosine is a 5′-methylcytosine, and wherein administration of the antisense oligonucleotide treats diabetes, obesity, metabolic syndrome, diabetic dyslipidemia, or hypertriglyceridemia in the animal.

In certain embodiments, a therapeutically effective amount of the compound administered to an animal reduces metabolic disease in the animal. In certain embodiments, the metabolic disease is obesity, diabetes, hyperglycemia, prediabetes, non-alcoholic fatty liver disease (NAFLD), metabolic syndrome, insulin resistance, diabetic dyslipidemia, or hypertriglyceridemia or a combination thereof. The NAFLD can be hepatic steatosis or steatohepatitis. The diabetes can be type 2 diabetes or type 2 diabetes with dyslipidemia.

Certain embodiments provide a method of increasing insulin sensitivity or hepatic insulin sensitivity in an animal comprising administering to the animal a compound comprising the modified oligonucleotide targeting PEPCK-M described herein.

Certain embodiments provide a method of reducing obesity, adipose tissue size or weight, body fat, glucose, glucose resistance, insulin resistance, triglyceride levels, or any combination thereof in an animal comprising administering to the animal a compound comprising the modified oligonucleotide targeted to PEPCK-M described herein.

In certain embodiments, PEPCK-M has the sequence as set forth in any of the GenBank Accession Numbers listed in Table 1 (incorporated herein as SEQ ID NOs: 1-5). In certain embodiments, PEPCK-M has the human sequence as set forth in GenBank Accession No. NM_{—}004563.2 (incorporated herein as SEQ ID NO: 1). In certain embodiments, PEPCK-M has the human sequence as set forth in nucleotides 5560000 to 5576000 of GenBank Accession No. NT_{—}026437.11 (incorporated herein as SEQ ID NO: 2). In certain embodiments, PEPCK-M has the human mRNA sequence as set forth in GenBank Accession No. X92720.1 (incorporated herein as SEQ ID NO: 3).

TABLE 1
Gene Target Names and Sequences

| | | | SEQ ID |
|---|---|---|---|
| Target Name | Species | Genbank # | NO |
| PEPCK-M | Human | NM_004563.2 | 1 |
| PEPCK-M | Human | nucleotides 5560000 | 2 |
| | | to 5576000 of | |
| | | NT_026437.11 | |
| PEPCK-M | Human | X92720.1 | 3 |
| PEPCK-M | Rat | XM_001055522.1 | 4 |
| PEPCK-M | Rat | nucleotides 5520000 | 5 |
| | | to 5546000 of | |
| | | NW_047454.2 | |

In certain embodiments, the animal is a human.

In certain embodiments, the compounds or compositions for the use in the methods provided herein are administered with a pharmaceutically acceptable carrier or diluent.

In certain embodiments, the compounds or compositions for the use in the methods provided herein are designated as a first agent. In certain embodiments, the methods for the use in the methods provided herein comprise administering a first and second agent. In certain embodiments, the first agent and the second agent are co-administered. In certain embodiments the first agent and the second agent are co-administered sequentially or concomitantly.

In certain embodiments, the second agent is a glucose-lowering agent. The glucose lowering agent can include, but is not limited to, a therapeutic lifestyle change, PPAR agonist, a dipeptidyl peptidase (IV) inhibitor, a GLP-1 analog, insulin or an insulin analog, an insulin secretagogue, a SGLT2 inhibitor, a human amylin analog, a biguanide, an alpha-glucosidase inhibitor, or a combination thereof. The glucose-lowering agent can include, but is not limited to metformin, sulfonylurea, rosiglitazone, meglitinide, thiazolidinedione, alpha-glucosidase inhibitor or a combination thereof. The sulfonylurea can be acetohexamide, chlorpropamide, tolbutamide, tolazamide, glimepiride, a glipizide, a glyburide, or a gliclazide. The meglitinide can be nateglinide or repaglinide. The thiazolidinedione can be pioglitazone or rosiglitazone. The alpha-glucosidase can be acarbose or miglitol.

In certain embodiments, the second agent is a lipid lowering therapy. In certain embodiments, the second agent is a LDL lowering therapy. In certain embodiments, the second agent is a triglyceride lowering therapy. In certain embodiments, the second agent is a cholesterol lowering therapy. In certain embodiments the lipid lowering therapy can include, but is not limited to, a therapeutic lifestyle change, statins, fibrates or MTP inhibitors.

In certain embodiments, administration comprises parenteral administration.

Certain embodiments provide the use of a compound as described herein for reducing PEPCK-M in an animal. In certain embodiments, the compound comprises a modified oligonucleotide 10 to 30 linked nucleosides in length targeted to PEPCK-M as shown in any of SEQ ID NOs: 1-3.

Certain embodiments provide the use of a compound as described herein for increasing insulin sensitivity in an animal. In certain embodiments, the compound comprises a modified oligonucleotide 10 to 30 linked nucleosides in length targeted to PEPCK-M as shown in any of SEQ ID NOs: 1-3.

Certain embodiments provide the use of a compound as described herein for reducing insulin levels, glucose levels, triglyceride levels, or adipose tissue size or weight in an animal. In certain embodiments, the compound comprises a modified oligonucleotide 10 to 30 linked nucleosides in length targeted to PEPCK-M as shown in any of SEQ ID NOs: 1-3.

Certain embodiments provide the use of a compound as described herein for treating, ameliorating, delaying or preventing one or more of a metabolic disease or a symptom thereof, in an animal. In certain embodiments, the compound comprises a modified oligonucleotide 10 to 30 linked nucleosides in length targeted to PEPCK-M as shown in any of SEQ ID NOs: 1-3.

Certain embodiments provide the use of a compound as described herein in the manufacture of a medicament for treating, ameliorating, delaying or preventing one or more of a metabolic disease or a symptom thereof. In certain embodiments, the compound comprises a modified oligonucleotide 10 to 30 linked nucleosides in length targeted to PEPCK-M as shown in any of SEQ ID NOs: 1-3.

Certain embodiments provide a kit for treating, preventing, or ameliorating one or more of a metabolic disease or a symptom thereof, as described herein wherein the kit comprises: a) a compound as described herein; and optionally b) an additional agent or therapy as described herein. The kit can further include instructions or a label for using the kit to treat, prevent, or ameliorate one or more of a metabolic disease or a symptom thereof.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to a PEPCK-M nucleic acid is 10 to 30 nucleotides in length. In other words, antisense compounds are from 10 to 30 linked nucleobases. In other embodiments, the antisense compound comprises a modified oligonucleotide consisting of 8 to 80, 12 to 50, 10 to 30, 12 to 30, 15 to 30, 18 to 24, 18 to 21, 19 to 22, or 20 linked nucleobases. In certain such embodiments, the antisense compound comprises a modified oligonucleotide consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked nucleobases in length, or a range defined by any two of the above values.

In certain embodiments, the antisense compound comprises a shortened or truncated modified oligonucleotide. The shortened or truncated modified oligonucleotide can have a single nucleoside deleted from the 5′ end (5′ truncation), or alternatively from the 3′ end (3′ truncation). A shortened or truncated oligonucleotide may have two nucleosides deleted from the 5′ end, or alternatively may have two subunits deleted from the 3′ end. Alternatively, the deleted nucleosides may be dispersed throughout the modified oligonucleotide, for example, in an antisense compound having one nucleoside deleted from the 5′ end and one nucleoside deleted from the 3′ end.

When a single additional nucleoside is present in a lengthened oligonucleotide, the additional nucleoside may be located at the 5′ or 3′ end of the oligonucleotide. When two or more additional nucleosides are present, the added nucleosides may be adjacent to each other, for example, in an oligonucleotide having two nucleosides added to the 5′ end (5′ addition), or alternatively to the 3′ end (3′ addition), of the oligonucleotide. Alternatively, the added nucleoside may be dispersed throughout the antisense compound, for example, in an oligonucleotide having one nucleoside added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to a PEPCK-M nucleic acid have chemically modified subunits arranged in patterns, or motifs, to confer to the antisense compounds properties such as enhanced the inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH₂)ₙ—O-2′ bridge, where n=1 or n=2). Preferably, each distinct region comprises uniform sugar moieties. The wing-gap-wing motif is frequently described as “X—Y—Z”, where “X” represents the length of the 5′ wing region, “Y” represents the length of the gap region, and “Z” represents the length of the 3′ wing region. As used herein, a gapmer described as “X—Y—Z” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30 or more nucleotides. Thus, gapmers include, but are not limited to, for example 5-10-5, 4-8-4, 4-12-3, 4-12-4, 3-14-3, 2-13-5, 2-16-2, 1-18-1, 3-10-3, 2-10-2, 1-10-1, 2-8-2, 6-8-6 or 5-8-5.

In certain embodiments, the antisense compound as a “wingmer” motif, having a wing-gap or gap-wing configuration, i.e. an X—Y or Y—Z configuration as described above for the gapmer configuration. Thus, wingmer configurations include, but are not limited to, for example 5-10, 8-4, 4-12, 12-4, 3-14, 16-2, 18-1, 10-3, 2-10, 1-10, 8-2, 2-13, or 5-13.

In certain embodiments, antisense compounds targeted to a PEPCK-M nucleic acid possess a 5-10-5 gapmer motif.

In certain embodiments, antisense compounds targeted to a PEPCK-M nucleic acid possess a 6-8-6 gapmer motif.

In certain embodiments, antisense compounds targeted to a PEPCK-M nucleic acid possess a 5-8-5 gapmer motif.

In certain embodiments, an antisense compound targeted to a PEPCK-M nucleic acid has a gap-widened motif.

In certain embodiments, a gap-widened antisense oligonucleotide targeted to a PEPCK-M nucleic acid has a gap segment of ten 2′-deoxyribonucleotides positioned immediately adjacent to and between wing segments of five chemically modified nucleosides. In certain embodiments, the chemical modification comprises a 2′-sugar modification. In another embodiment, the chemical modification comprises a 2′-MOE sugar modification.

In certain embodiments, a gap-widened antisense oligonucleotide targeted to a PEPCK-M nucleic acid has a gap segment of eight 2′-deoxyribonucleotides positioned immediately adjacent to and between wing segments of five chemically modified nucleosides. In certain embodiments, the chemical modification comprises a 2′-sugar modification. In another embodiment, the chemical modification comprises a 2′-MOE sugar modification.

In certain embodiments, a gap-widened antisense oligonucleotide targeted to a PEPCK-M nucleic acid has a gap segment of eight 2′-deoxyribonucleotides positioned immediately adjacent to and between wing segments of six chemically modified nucleosides. In certain embodiments, the chemical modification comprises a 2′-sugar modification. In another embodiment, the chemical modification comprises a 2′-MOE sugar modification.

### Target Nucleic Acids, Target Regions and Nucleotide Sequences

In certain embodiments, the PEPCK-M nucleic acid is any of the sequences set forth in GENBANK Accession No. NM_{—}004563.2, first deposited with GENBANK® on May 19, 2005 (incorporated herein as SEQ ID NO: 1), GENBANK Accession No. NT_{—}026437.11 truncated from nucleotides 5560000 to 5576000, first deposited with GENBANK® on Mar. 1, 2006 (incorporated herein as SEQ ID NO: 2); GENBANK Accession No. X92720.1 first deposited with GENBANK® on Nov. 2, 1995 (incorporated herein as SEQ ID NO: 3); GENBANK Accession No. XM_{—}001055522.1 first deposited with GENBANK® on Jun. 22, 2006 (incorporated herein as SEQ ID NO: 4); and GENBANK Accession No. NW_{—}047454.2 truncated from nucleotides 5520000 to 5546000 (incorporated herein as SEQ ID NO: 5), first deposited with GENBANK® on Apr. 15, 2005.

It is understood that the sequence set forth in each SEQ ID NO in the Examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for PEPCK-M can be obtained by accession number from sequence databases such as NCBI and such information is incorporated herein by reference. In certain embodiments, a target region may encompass the sequence from a 5′ target site of one target segment within the target region to a 3′ target site of another target segment within the target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within an active target region. In certain embodiments, reductions in PEPCK-M mRNA levels are indicative of inhibition of PEPCK-M expression. Reductions in levels of a PEPCK-M protein are also indicative of inhibition of target mRNA expression. Further, phenotypic changes are indicative of inhibition of PEPCK-M expression. For example, improvement in insulin sensitivity, improvement in metabolic rate, decrease in glucose levels, decrease in insulin levels, decrease in hepatic glycogen production, decrease in triglyceride levels, decrease in body weight, or decrease in body fat among other phenotypic changes that may be assayed. Other phenotypic indications, e.g., symptoms associated with metabolic diseases, may also be assessed as described below.

### Hybridization

In some embodiments, hybridization occurs between an antisense compound disclosed herein and a PEPCK-M nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a PEPCK-M nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a PEPCK-M nucleic acid).

An antisense compound may hybridize over one or more segments of a PEPCK-M nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a PEPCK-M nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

In certain embodiments, the antisense compounds provided herein, or specified portions thereof, are fully complementary (i.e. 100% complementary) to a target nucleic acid, or specified portion thereof. For example, antisense compound may be fully complementary to a PEPCK-M nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In one embodiment, a non-complementary nucleobase is located in the wing segment of a gapmer antisense oligonucleotide.

In certain embodiments, antisense compounds that are, or are up to 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a PEPCK-M nucleic acid, or specified portion thereof.

In certain embodiments, antisense compounds that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a PEPCK-M nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a PEPCK-M nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds for the use in the methods provided herein can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH₃, 2′-OCH₂CH₃, 2′-OCH₂CH₂F and 2′—O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ), where each R₁, Rₘ and Rᵢᵢ is, independently, H or substituted or unsubstituted C₁-C₁₀ alkyl.

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—S-2; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C—(H)(CH₃)-2′ (see Chattopadhyaya et al., J. Org. Chem., 2009, 74, 118-134); and 4′-CH₂—C(═CH₂)-2′ (and analogs thereof see published International Application WO 2008/154401, published on Dec. 8, 2008).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. Patent Ser. Nos. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R)-2′ and 4′-CH₂—N(R)—O-2′- wherein each R is, independently, H, a protecting group or C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-methyleneoxy (4′-CH₂—O-2′) BNA, (B) β-D-methyleneoxy (4′-CH₂—O-2′) BNA, (C) ethyleneoxy (4′-(CH₂)₂—O-2′) BNA, (D) aminooxy (4′-CH₂—O—N(R)-2′) BNA, (E) oxyamino (4′-CH₂—N(R)—O-2′) BNA, and (F) methyl(methyleneoxy) (4′-CH(CH₃)—O-2′) BNA, (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, and (J) propylene carbocyclic (4′-(CH₂)₃-2′) BNA as depicted below.

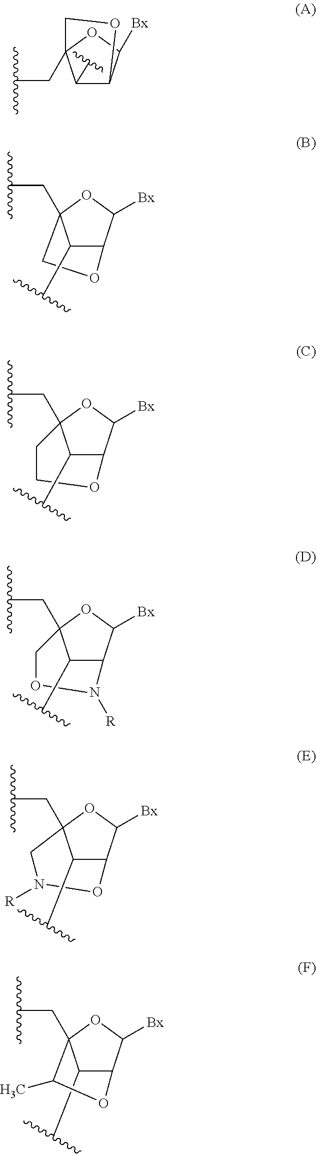

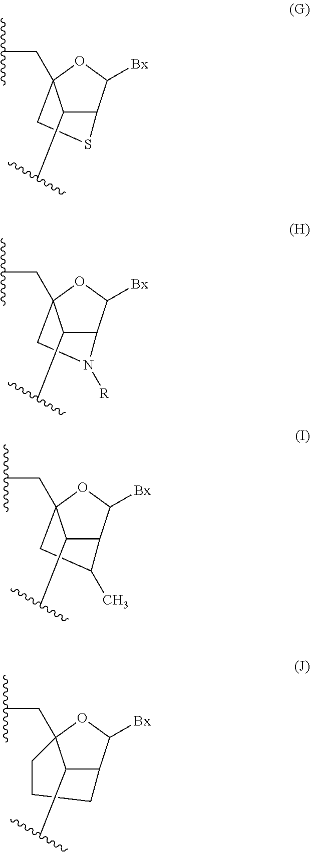

wherein Bx is the base moiety and R is independently H, a protecting group or C₁-C₁₂ alkyl.

In certain embodiments, bicyclic nucleosides are provided having Formula I:

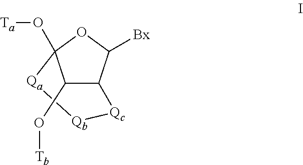

wherein:

Bx is a heterocyclic base moiety;

-Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;

R_{c} is C₁-C₁₂ alkyl or an amino protecting group; and

Tₐ and T_{b} are each, independently H, a hydroxyl protecting group, a conjugate group, a reactive phosphorus group, a phosphorus moiety or a covalent attachment to a support medium.

In certain embodiments, bicyclic nucleosides are provided having Formula II:

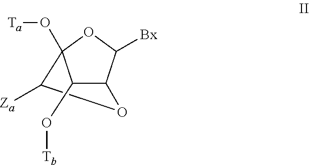

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups is, independently, mono or poly substituted with substituent groups independently selected from halogen, oxo, hydroxyl, OJ_{c}, NJ_{c}J_{d}, SJ_{c}, N₃, OC(═X)J_{c}, and NJₑC(═X)NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleosides are provided having Formula III:

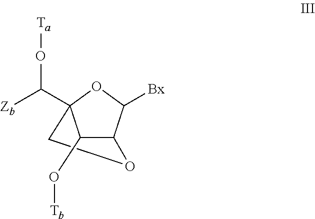

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleosides are provided having Formula IV:

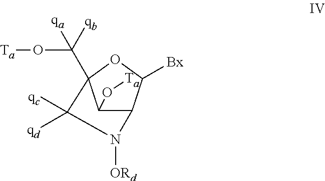

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleosides are provided having Formula V:

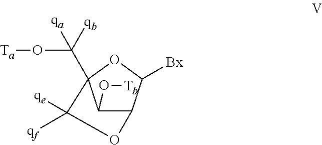

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;

or qₑ and q_{f} together are ═C(q_{g})(qₕ);

q_{g} and qₕ are each, independently, H, halogen, C₁-C₁₂ alkyl or substituted C₁-C₁₂ alkyl.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleosides are provided having Formula VI:

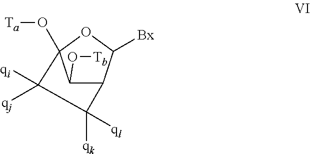

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and

qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129(26), 8362-8379).

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙF, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, F, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties. In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, a “modified tetrahydropyran nucleoside” or “modified THP nucleoside” means a nucleoside having a six-membered tetrahydropyran “sugar” substituted in for the pentofuranosyl residue in normal nucleosides (a sugar surrogate). Modified THP nucleosides include, but are not limited to, what is referred to in the art as hexitol nucleic acid (HNA), anitol nucleic acid (ANA), manitol nucleic acid (MNA) (see Leumann, Bioorg. Med. Chem., 2002, 10, 841-854), fluoro HNA (F—HNA) or those compounds having Formula VII:

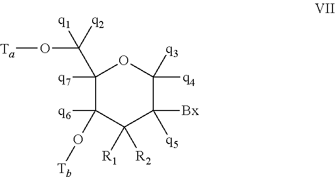

wherein independently for each of said at least one tetrahydropyran nucleoside analog of Formula VII:

Bx is a heterocyclic base moiety;

Tₐ and T_{b} are each, independently, an internucleoside linking group linking the tetrahydropyran nucleoside analog to the antisense compound or one of Tₐ and T_{b} is an internucleoside linking group linking the tetrahydropyran nucleoside analog to the antisense compound and the other of Tₐ and T_{b} is H, a hydroxyl protecting group, a linked conjugate group or a 5′ or 3′-terminal group;

q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and each of R₁ and R₂ is selected from hydrogen, hydroxyl, halogen, subsitituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is fluoro. In certain embodiments, R₁ is fluoro and R₂ is H; R₁ is methoxy and R₂ is H, and R₁ is H and R₂ is methoxyethoxy.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′ substituents, such as allyl, amino, azido, thio, O-allyl, O—C₁-C₁₀ alkyl, —OCF₃, O—(CH₂)₂—O—CH₃, 2′—O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modified nucleosides may further comprise other modifications, for example at other positions of the sugar and/or at the nucleobase.

As used herein, “2′-F” refers to a nucleoside comprising a sugar comprising a fluoro group at the 2′ position.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

Many other bicyclo and tricyclo sugar surrogate ring systems are also known in the art that can be used to modify nucleosides for incorporation into antisense compounds (see for example review article: Leumann, Bioorg. Med. Chem., 2002, 10, 841-854).

Such ring systems can undergo various additional substitutions to enhance activity.

Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense compounds comprise one or more nucleosides having modified sugar moieties. In certain embodiments, the modified sugar moiety is 2′-MOE. In certain embodiments, the 2′-MOE modified nucleosides are arranged in a gapmer motif. In certain embodiments, the modified sugar moiety is a bicyclic nucleoside having a (4′-CH(CH₃)—O-2′) bridging group. In certain embodiments, the (4′-CH(CH₃)—O-2′) modified nucleosides are arranged throughout the wings of a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2 aminopropyladenine, 5-propynyluracil and 5-propynylcytosine.

In certain embodiments, antisense compounds targeted to a PEPCK-M nucleic acid comprise one or more modified nucleobases. In certain embodiments, gap-widened antisense oligonucleotides targeted to a PEPCK-M nucleic acid comprise one or more modified nucleobases. In certain embodiments, the modified nucleobase is 5-methylcytosine. In certain embodiments, each cytosine is a 5-methylcytosine.

### Compositions and Methods for Formulating Pharmaceutical Compositions

Antisense oligonucleotides can be admixed with pharmaceutically acceptable active or inert substance for the preparation of pharmaceutical compositions or formulations. Compositions and methods for the formulation of pharmaceutical compositions are dependent upon a number of criteria, including, but not limited to, route of administration, extent of disease, or dose to be administered.

Antisense compound targeted to a PEPCK-M nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to a PEPCK-M nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds can be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of PEPCK-M nucleic acids can be tested in vitro in a variety of cell types. Cell types used for such analyses are available from commercial vendors (e.g. American Type Culture Collection, Manassus, Va.; Zen-Bio, Inc., Research Triangle Park, N.C.; Clonetics Corporation, Walkersville, Md.) and cells are cultured according to the vendor's instructions using commercially available reagents (e.g. Invitrogen Life Technologies, Carlsbad, Calif.). Illustrative cell types include, but are not limited to, HepG2 cells, Hep3B cells, Huh7 (hepatocellular carcinoma) cells, primary hepatocytes, A549 cells, GM04281 fibroblasts and LLC-MK2 cells.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluence in culture.

One reagent commonly used to introduce antisense oligonucleotides into cultured cells includes the cationic lipid transfection reagent LIPOFECTIN® (Invitrogen, Carlsbad, Calif.). Antisense oligonucleotides are mixed with LIPOFECTIN® in OPTI-MEM® 1 (Invitrogen, Carlsbad, Calif.) to achieve the desired final concentration of antisense oligonucleotide and a LIPOFECTIN® concentration that typically ranges 2 to 12 ug/mL per 100 nM antisense oligonucleotide.

Another reagent used to introduce antisense oligonucleotides into cultured cells includes LIPOFECTAMINE 2000® (Invitrogen, Carlsbad, Calif.). Antisense oligonucleotide is mixed with LIPOFECTAMINE 2000® in OPTI-MEM® 1 reduced serum medium (Invitrogen, Carlsbad, Calif.) to achieve the desired concentration of antisense oligonucleotide and a LIPOFECTAMINE® concentration that typically ranges 2 to 12 ug/mL per 100 nM antisense oligonucleotide.

Another reagent used to introduce antisense oligonucleotides into cultured cells includes Cytofectin® (Invitrogen, Carlsbad, Calif.). Antisense oligonucleotide is mixed with Cytofectin® in OPTI-MEM® 1 reduced serum medium (Invitrogen, Carlsbad, Calif.) to achieve the desired concentration of antisense oligonucleotide and a Cytofectin® concentration that typically ranges 2 to 12 ug/mL per 100 nM antisense oligonucleotide.

Another reagent used to introduce antisense oligonucleotides into cultured cells includes Oligofectamine™ (Invitrogen Life Technologies, Carlsbad, Calif.). Antisense oligonucleotide is mixed with Oligofectamine™ in Opti-MEM™-1 reduced serum medium (Invitrogen Life Technologies, Carlsbad, Calif.) to achieve the desired concentration of oligonucleotide with an Oligofectamine™ to oligonucleotide ratio of approximately 0.2 to 0.8 μL per 100 nM.

Another reagent used to introduce antisense oligonucleotides into cultured cells includes FuGENE 6 (Roche Diagnostics Corp., Indianapolis, Ind.). Antisense oligomeric compound was mixed with FuGENE 6 in 1 mL of serum-free RPMI to achieve the desired concentration of oligonucleotide with a FuGENE 6 to oligomeric compound ratio of 1 to 4 μL of FuGENE 6 per 100 nM.

Another technique used to introduce antisense oligonucleotides into cultured cells includes electroporation (Sambrooke and Russell, Molecular Cloning: A Laboratory Manual, 3^{rd} Ed., 2001).

Cells are treated with antisense oligonucleotides by routine methods. Cells are typically harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line.

Methods to determine the optimal antisense oligonucleotide concentration for a particular cell line are well known in the art. Antisense oligonucleotides are typically used at concentrations ranging from 1 nM to 300 nM when transfected with LIPOFECTAMINE2000® (Invitrogen, Carlsbad, Calif.), Lipofectin® (Invitrogen, Carlsbad, Calif.) or Cytofectin™ (Genlantis, San Diego, Calif.). Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art. RNA is prepared using methods well known in the art, for example, using the TRIZOL® Reagent (Invitrogen, Carlsbad, Calif.) according to the manufacturer's recommended protocols.

### Analysis of Inhibition of Target Levels or Expression

Inhibition of levels or expression of a PEPCK-M nucleic acid can be assayed in a variety of ways known in the art. For example, target nucleic acid levels can be quantitated by, e.g., Northern blot analysis, competitive polymerase chain reaction (PCR), or quantitaive real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM® 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels can be accomplished by quantitative real-time PCR using the ABI PRISM® 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT, real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN® (Invitrogen, Inc. Carlsbad, Calif.). Cyclophilin A expression is quantified by real time PCR, by being run simultaneously with the target, multiplexing, or separately. Total RNA is quantified using RIBOGREEN® RNA quantification reagent (Invitrogen, Inc. Eugene, Oreg.). Methods of RNA quantification by RIBOGREEN® are taught in Jones, L. J., et al, (Analytical Biochemistry, 1998, 265, 368-374). A CYTOFLUOR® 4000 instrument (PE Applied Biosystems) is used to measure RIBOGREEN® fluorescence.

Probes and primers are designed to hybridize to a PEPCK-M nucleic acid. Methods for designing real-time PCR probes and primers are well known in the art, and can include the use of software such as PRIMER EXPRESS® Software (Applied Biosystems, Foster City, Calif.).

Gene target quantities obtained by RT, real-time PCR were normalized using either the expression level of GAPDH or Cyclophilin A, genes whose expression are constant, or by quantifying total RNA using RiboGreen™ (Molecular Probes, Inc. Eugene, Oreg.). GAPDH or Cyclophilin A expression can be quantified by RT, real-time PCR, by being run simultaneously with the target, multiplexing, or separately. Total RNA was quantified using RiboGreen™ RNA quantification reagent (Molecular Probes, Inc. Eugene, Oreg.).

### Analysis of Protein Levels

Antisense inhibition of PEPCK-M nucleic acids can be assessed by measuring PEPCK-M protein levels. Protein levels of PEPCK-M can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art.

### In Vivo Testing of Antisense Compounds

Antisense compounds, for example, antisense oligonucleotides, are tested in animals to assess their ability to inhibit expression of PEPCK-M and produce phenotypic changes. Testing can be performed in normal animals, or in experimental disease models. For administration to animals, antisense oligonucleotides are formulated in a pharmaceutically acceptable diluent, such as phosphate-buffered saline. Administration includes parenteral routes of administration. Following a period of treatment with antisense oligonucleotides, RNA is isolated from tissue and changes in PEPCK-M nucleic acid expression are measured. Changes in PEPCK-M protein levels are also measured.

### Certain Indications

In certain embodiments, provided herein are methods of treating an individual comprising administering one or more pharmaceutical compositions as described herein. In certain embodiments, the individual has a metabolic disease.

Accordingly, provided herein are methods for ameliorating a metabolic disease in a subject in need thereof. In certain embodiments, provided is a method for reducing the rate of onset of a symptom associated with a metabolic disease. In certain embodiments, provided is a method for reducing the severity of a symptom associated with metabolic disease. In such embodiments, the methods comprise administering to an individual in need thereof a therapeutically effective amount of a compound targeted to a PEPCK-M nucleic acid. In certain embodiments, the metabolic disease is diabetes, obesity, metabolic syndrome, diabetic dyslipidemia, or hypertriglyceridemia.

Also, provided herein are methods for ameliorating a symptom associated with metabolic disease in a subject in need thereof. In certain embodiments, provided is a method for reducing the rate of onset of a symptom associated with metabolic disease. In certain embodiments, provided is a method for reducing the severity of a symptom associated with metabolic disease. In such embodiments, the methods comprise administering to an individual in need thereof a therapeutically effective amount of a compound targeted to a PEPCK-M nucleic acid.

In certain embodiments, administration of an antisense compound targeted to a PEPCK-M nucleic acid results in reduction of PEPCK-M expression by at least about 15, 20, 25, 30, 35, 40, 45, 50, 55, 60, 65, 70, 75, 80, 85, 90, 95 or 99%, or a range defined by any two of these values.

In certain embodiments, pharmaceutical compositions comprising an antisense compound targeted to PEPCK-M are used for the preparation of a medicament for treating a patient suffering or susceptible to metabolic disease.

In certain embodiments, the methods described herein include administering a compound comprising a modified oligonucleotide having an 8, 9, 10, 11, 12, 13, 14, 15, 16, 17, 18, 19 or 20 contiguous nucleobase portion.

In certain embodiments, the methods described herein include methods for ameliorating a metabolic disease in an animal comprising administering to the animal a therapeutically effective amount of a compound comprising an antisense oligonucleotide consisting of 10 to 30 linked nucleosides in length targeted to PEPCK-M.

In certain embodiments, the methods described herein include methods for ameliorating a metabolic disease in an animal comprising administering to the animal a therapeutically effective amount of a compound comprising an antisense oligonucleotide consisting of 10 to 30 linked nucleosides in length targeted to PEPCK-M.

### Administration

In certain embodiments, the compounds and compositions as described herein may be administered in a number of ways depending upon whether local or systemic treatment is desired and upon the area to be treated. Administration may be topical, pulmonary, e.g., by inhalation or insufflation of powders or aerosols, including by nebulizer; intratracheal, intranasal, epidermal and transdermal, oral or parenteral. The compounds and compositions as described herein can be administered directly to a tissue or organ.

In certain embodiments, the compounds and compositions as described herein are administered parenterally. “Parenteral administration” means administration through injection or infusion. Parenteral administration includes subcutaneous administration, intravenous administration, intramuscular administration, intraarterial administration, intraperitoneal administration, or intracranial administration, e.g. intracerebral administration, intrathecal administration, intraventricular administration, ventricular administration, intracerebroventricular administration, cerebral intraventricular administration or cerebral ventricular administration. Administration can be continuous, or chronic, or short or intermittent.

In certain embodiments, parenteral administration is by infusion. Infusion can be chronic or continuous or short or intermittent. In certain embodiments, infused pharmaceutical agents are delivered with a pump.

In certain embodiments, parenteral administration is by injection. The injection can be delivered with a syringe or a pump. In certain embodiments, the injection is a bolus injection. In certain embodiments, the injection is administered directly to a tissue or organ.

In certain embodiments, the compounds and compositions as described herein are administered parenterally.

In certain embodiments, parenteral administration is subcutaneous.

In further embodiments, the formulation for administration is the compounds described herein and saline.

In certain embodiments, an antisense oligonucleotide is delivered by injection or infusion once every month, every two months, every 90 days, every 3 months, every 6 months, twice a year or once a year.

### Certain Combination Therapies

In certain embodiments, one or more pharmaceutical compositions of the present invention are co-administered with one or more other pharmaceutical agents. In certain embodiments, such one or more other pharmaceutical agents are designed to treat the same disease, disorder, or condition as the one or more pharmaceutical compositions described herein. In certain embodiments, such one or more other pharmaceutical agents are designed to treat a different disease, disorder, or condition as the one or more pharmaceutical compositions described herein. In certain embodiments, such one or more other pharmaceutical agents are designed to treat an undesired side effect of one or more pharmaceutical compositions as described herein. In certain embodiments, one or more pharmaceutical compositions are co-administered with another pharmaceutical agent to treat an undesired effect of that other pharmaceutical agent. In certain embodiments, one or more pharmaceutical compositions are co-administered with another pharmaceutical agent to produce a combinational effect. In certain embodiments, one or more pharmaceutical compositions are co-administered with another pharmaceutical agent to produce a synergistic effect.

In certain embodiments, a first agent and one or more second agents are administered at the same time. In certain embodiments, the first agent and one or more second agents are administered at different times. In certain embodiments, the first agent and one or more second agents are prepared together in a single pharmaceutical formulation. In certain embodiments, the first agent and one or more second agents are prepared separately.

In certain embodiments, the second compound is administered prior to administration of a pharmaceutical composition of the present invention. In certain embodiments, the second compound is administered following administration of a pharmaceutical composition of the present invention. In certain embodiments, the second compound is administered at the same time as a pharmaceutical composition of the present invention. In certain embodiments, the dose of a co-administered second compound is the same as the dose that would be administered if the second compound was administered alone. In certain embodiments, the dose of a co-administered second compound is lower than the dose that would be administered if the second compound was administered alone. In certain embodiments, the dose of a co-administered second compound is greater than the dose that would be administered if the second compound was administered alone.

In certain embodiments, the co-administration of a second compound enhances the effect of a first compound, such that co-administration of the compounds results in an effect that is greater than the effect of administering the first compound alone. In certain embodiments, the co-administration results in effects that are additive of the effects of the compounds when administered alone. In certain embodiments, the co-administration results in effects that are supra-additive of the effects of the compounds when administered alone. In certain embodiments, the first compound is an antisense compound. In certain embodiments, the second compound is an antisense compound.

In certain embodiments, second agents include, but are not limited to, a glucose-lowering agent. The glucose lowering agent can include, but is not limited to, a therapeutic lifestyle change, PPAR agonist, a dipeptidyl peptidase (IV) inhibitor, a GLP-1 analog, insulin or an insulin analog, an insulin secretagogue, a SGLT2 inhibitor, a human amylin analog, a biguanide, an alpha-glucosidase inhibitor, or a combination thereof. The glucose-lowering agent can include, but is not limited to metformin, sulfonylurea, rosiglitazone, meglitinide, thiazolidinedione, alpha-glucosidase inhibitor or a combination thereof. The sulfonylurea can be acetohexamide, chlorpropamide, tolbutamide, tolazamide, glimepiride, a glipizide, a glyburide, or a gliclazide. The meglitinide can be nateglinide or repaglinide. The thiazolidinedione can be pioglitazone or rosiglitazone. The alpha-glucosidase can be acarbose or miglitol.

In some embodiments, the glucose-lowering therapeutic is a GLP-1 analog. In some embodiments, the GLP-1 analog is exendin-4 or liraglutide.

In other embodiments, the glucose-lowering therapeutic is a sulfonylurea. In some embodiments, the sulfonylurea is acetohexamide, chlorpropamide, tolbutamide, tolazamide, glimepiride, a glipizide, a glyburide, or a gliclazide.

In some embodiments, the glucose-lowering drug is a biguanide. In some embodiments, the biguanide is metformin, and in some embodiments, blood glucose levels are decreased without increased lactic acidosis as compared to the lactic acidosis observed after treatment with metformin alone.

In some embodiments, the glucose-lowering drug is a meglitinide. In some embodiments, the meglitinide is nateglinide or repaglinide.

In some embodiments, the glucose-lowering drug is a thiazolidinedione. In some embodiments, the thiazolidinedione is pioglitazone, rosiglitazone, or troglitazone. In some embodiments, blood glucose levels are decreased without greater weight gain than observed with rosiglitazone treatment alone.

In some embodiments, the glucose-lowering drug is an alpha-glucosidase inhibitor. In some embodiments, the alpha-glucosidase inhibitor is acarbose or miglitol.

In a certain embodiment, a co-administered glucose-lowering agent is ISIS 113715.

In a certain embodiment, glucose-lowering therapy is therapeutic lifestyle change.

In certain embodiments, second agents include, but are not limited to, lipid-lowering agents. The lipid-lowering agent can include, but is not limited to atorvastatin, simvastatin, rosuvastatin, and ezetimibe. In certain such embodiments, the lipid-lowering agent is administered prior to administration of a pharmaceutical composition of the present invention. In certain such embodiments, the lipid-lowering agent is administered following administration of a pharmaceutical composition of the present invention. In certain such embodiments the lipid-lowering agent is administered at the same time as a pharmaceutical composition of the present invention. In certain such embodiments the dose of a co-administered lipid-lowering agent is the same as the dose that would be administered if the lipid-lowering agent was administered alone. In certain such embodiments the dose of a co-administered lipid-lowering agent is lower than the dose that would be administered if the lipid-lowering agent was administered alone. In certain such embodiments the dose of a co-administered lipid-lowering agent is greater than the dose that would be administered if the lipid-lowering agent was administered alone.

In certain embodiments, a co-administered lipid-lowering agent is a HMG-CoA reductase inhibitor. In certain such embodiments the HMG-CoA reductase inhibitor is a statin. In certain such embodiments the statin is selected from atorvastatin, simvastatin, pravastatin, fluvastatin, and rosuvastatin.

In certain embodiments, a co-administered lipid-lowering agent is a cholesterol absorption inhibitor. In certain such embodiments, cholesterol absorption inhibitor is ezetimibe.

In certain embodiments, a co-administered lipid-lowering agent is a co-formulated HMG-CoA reductase inhibitor and cholesterol absorption inhibitor. In certain such embodiments the co-formulated lipid-lowering agent is ezetimibe/simvastatin.

In certain embodiments, a co-administered lipid-lowering agent is a microsomal triglyceride transfer protein inhibitor (MTP inhibitor).

In certain embodiments, a co-administered lipid-lowering agent is an oligonucleotide targeted to ApoB.

In certain embodiments, second agents include, but are not limited to an anti-obesity drug or agent. Such anti-obesity agents include but are not limited to Orlistat, Sibutramine, or Rimonabant, and may be administered as described above as adipose or body weight lowering agents. In certain embodiments, the antisense compound may be co-administered with appetite suppressants. Such appetite suppressants include but are not limited to diethylpropion tenuate, mazindol, orlistat, phendimetrazine, phentermine, and sibutramine and may be administered as described herein. In certain embodiment, the anti-obesity agents are CNS based such as, but not limited to, sibutramine or GLP-1 based such as, but not limited to, liraglutide.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

### Example 1

### Antisense Inhibition of Human Phosphoenolpyruvate Carboxykinase-Mitochondrial (PEPCK-M) in T-24 Cells

Antisense oligonucleotides targeted to a human PEPCK-M nucleic acid were tested for their effect on PEPCK-M RNA transcript in vitro. Cultured T-24 cells at a density of 20,000 cells per well were transfected using electroporation with 150 nM antisense oligonucleotide. After approximately 24 hours, RNA was isolated from the cells and PEPCK-M RNA transcript levels were measured by quantitative real-time PCR with human primer probe set RTS133 (forward sequence AGACCCTGCGAGTGCTTAGTG, designated herein as SEQ ID NO: 6; reverse sequence GATGTGGATGCCCTCTGGTT, designated herein as SEQ ID NO: 7; probe sequence CCAGCTTCCCACTGGCATTCGAGATTX, designated herein as SEQ ID NO: 8). PEPCK-M RNA transcript levels were adjusted according to total RNA content, as measured by RIBOGREEN®. Results are presented as percent inhibition of PEPCK-M, relative to untreated control cells.

The antisense oligonucleotides in Tables 2, 3, and 4 are uniform oligonucleotides or 5-10-5 gapmers, as indicated in the ‘Motif’ column. The uniform oligonucleotides have 2′-deoxyribose sugar residues and a phosphorothioate backbone. The 5-10⁻⁵ MOE gapmers are oligonucleotides where the gap segment comprises ten 2′-deoxynucleosides and each wing segment comprises five 2′-MOE nucleosides. The internucleoside linkages throughout each gapmer are phosphorothioate (P═S) linkages. All cytidine residues throughout each gapmer are 5-methylcytidines. ‘Target start site’ indicates the 5′-most nucleotide to which the antisense oligonucleotide is targeted. ‘Target stop site’indicates the 3′-most nucleotide to which the antisense oligonucleotide is targeted. All the antisense oligonucleotides listed in Table 2 target SEQ ID NO: 1 (GENBANK Accession No. NM_{—}004563.2). All the antisense oligonucleotides listed in Table 3 target SEQ ID NO: 2 (GENBANK Accession No. NT_{—}026437.11 truncated from nucleotides 5560000 to 5576000). All the antisense oligonucleotides listed in Table 4 target SEQ ID NO: 3 (GENBANK Accession No. X92720.1).

TABLE 2
Inhibition of human PEPCK-M RNA transcript in T24 cells
by antisense oligonucleotides targeting SEQ ID NO: 1

| | | Target | Target | | | SEQ |
|---|---|---|---|---|---|---|
| ISIS | | Start | Stop | | % | ID |
| No | Motif | Site | Site | Sequence | inhibition | NO. |
| 104129 | Uniform | 84 | 103 | AGGAACCGAGCGGAGCCGGG | 31 | 9 |
| 104130 | Uniform | 122 | 141 | TGCGGCCATGGCACCTGGGC | 44 | 10 |
| 104131 | Uniform | 132 | 151 | GGCGGTACAATGCGGCCATG | 18 | 11 |
| 104132 | Uniform | 191 | 210 | GCTACGGCATGATGGCCAGC | 0 | 12 |
| 104133 | Uniform | 245 | 264 | AATGCCAGTGGGAAGCTGGC | 0 | 13 |
| 104134 | Uniform | 308 | 327 | TCCATCACAGATGTGGATGC | 39 | 14 |
| 104135 | Uniform | 365 | 384 | TCGGATGAGGCCCTGCTGCT | 0 | 15 |
| 104136 | Uniform | 443 | 462 | CACCGTCTTGCTCTCTACTC | 58 | 16 |
| 104138 | Uniform | 572 | 591 | CTGCATGCAGCCTGGAAACC | 13 | 17 |
| 104139 | Uniform | 647 | 666 | GAGCTGCACCCCGATGCGGG | 0 | 18 |
| 104140 | Uniform | 696 | 715 | CCAGTCGGGTCATAATACGC | 24 | 19 |
| 104141 | Uniform | 742 | 761 | CACTTGACAAAGTCACCATC | 0 | 20 |
| 104142 | Uniform | 805 | 824 | TTGCACGGCCACTGGCTCAC | 4 | 21 |
| 104143 | Uniform | 852 | 871 | TGATCTCCCGCTGGTCGGGC | 24 | 22 |
| 104144 | Uniform | 896 | 915 | CTTGCCCAGCAGGGAGTTGC | 0 | 23 |
| 104145 | Uniform | 935 | 954 | CCGGGCCAGCCGAGAGGCGA | 0 | 24 |
| 104146 | Uniform | 980 | 999 | GGTGATGCCCAGGATCAGCA | 21 | 25 |
| 104147 | Uniform | 1028 | 1047 | GGCACTAGGGAAGGCGGCTG | 8 | 26 |
| 104148 | Uniform | 1077 | 1096 | TCCAGCCTGGCAGTGCAGGC | 23 | 27 |
| 104149 | Uniform | 1142 | 1161 | GGCCCGGAGTCGACCTTCAC | 32 | 28 |
| 104150 | Uniform | 1207 | 1226 | GCGTTGGGATTGGTGGTGGC | 18 | 29 |
| 104151 | Uniform | 1275 | 1294 | AGTACACGCCACCATCACTG | 6 | 30 |
| 104152 | Uniform | 1343 | 1362 | TTTCCAGGGTTTGCCCAGCC | 43 | 31 |
| 104153 | Uniform | 1434 | 1453 | GGGCCTCCCAGGCTGGGTCC | 0 | 32 |
| 104154 | Uniform | 1537 | 1556 | CCCACAAACACCCCATGACG | 65 | 33 |
| 104155 | Uniform | 1581 | 1600 | CTTTGTGTTCTGCTGCAGCA | 50 | 34 |
| 104156 | Uniform | 1646 | 1665 | GTAGTGCCCGAAGTTGTAGC | 20 | 35 |
| 104157 | Uniform | 1726 | 1745 | TCACGCCGGAACCAGTTGAC | 0 | 36 |
| 104158 | Uniform | 1770 | 1789 | GAGCATTCTCCCCAAAGCCT | 2 | 37 |
| 104159 | Uniform | 1830 | 1849 | TGGGTGTCTCTCGGGCACTG | 10 | 38 |
| 104160 | Uniform | 1875 | 1894 | TGAGGCCGCTGAGATCCAAG | 34 | 39 |
| 104161 | Uniform | 1939 | 1958 | TCACGAACCTCCTGTTCCCA | 41 | 40 |
| 104162 | Uniform | 1981 | 2000 | GGCAGATCCTGGTTGACCTG | 18 | 41 |
| 104163 | Uniform | 2036 | 2055 | TCACATTTTGTGCACACGTC | 24 | 42 |
| 104165 | Uniform | 2094 | 2113 | TGCCTTCCCTATTCCCAGAT | 19 | 43 |
| 104166 | Uniform | 2136 | 2155 | AAGATGTTAGTTAATATCAA | 0 | 44 |
| 104167 | Uniform | 2170 | 2189 | GGACAGTCTTTGTGGGAAGG | 0 | 45 |
| 104169 | 5-10-5 MOE | 84 | 103 | AGGAACCGAGCGGAGCCGGG | 75 | 9 |
| 104170 | 5-10-5 MOE | 122 | 141 | TGCGGCCATGGCACCTGGGC | 57 | 10 |
| 104171 | 5-10-5 MOE | 132 | 151 | GGCGGTACAATGCGGCCATG | 55 | 11 |
| 104172 | 5-10-5 MOE | 191 | 210 | GCTACGGCATGATGGCCAGC | 47 | 12 |
| 104173 | 5-10-5 MOE | 245 | 264 | AATGCCAGTGGGAAGCTGGC | 3 | 13 |
| 104174 | 5-10-5 MOE | 308 | 327 | TCCATCACAGATGTGGATGC | 79 | 14 |
| 104175 | 5-10-5 MOE | 365 | 384 | TCGGATGAGGCCCTGCTGCT | 47 | 15 |
| 104176 | 5-10-5 MOE | 443 | 462 | CACCGTCTTGCTCTCTACTC | 85 | 16 |
| 104178 | 5-10-5 MOE | 572 | 591 | CTGCATGCAGCCTGGAAACC | 65 | 17 |
| 104179 | 5-10-5 MOE | 647 | 666 | GAGCTGCACCCCGATGCGGG | 48 | 18 |
| 104180 | 5-10-5 MOE | 696 | 715 | CCAGTCGGGTCATAATACGC | 81 | 19 |
| 104181 | 5-10-5 MOE | 742 | 761 | CACTTGACAAAGTCACCATC | 45 | 20 |
| 104182 | 5-10-5 MOE | 805 | 824 | TTGCACGGCCACTGGCTCAC | 70 | 21 |
| 104183 | 5-10-5 MOE | 852 | 871 | TGATCTCCCGCTGGTCGGGC | 78 | 22 |
| 104184 | 5-10-5 MOE | 896 | 915 | CTTGCCCAGCAGGGAGTTGC | 1 | 23 |
| 104185 | 5-10-5 MOE | 935 | 954 | CCGGGCCAGCCGAGAGGCGA | 33 | 24 |
| 104186 | 5-10-5 MOE | 980 | 999 | GGTGATGCCCAGGATCAGCA | 18 | 25 |
| 104187 | 5-10-5 MOE | 1028 | 1047 | GGCACTAGGGAAGGCGGCTG | 62 | 26 |
| 104188 | 5-10-5 MOE | 1077 | 1096 | TCCAGCCTGGCAGTGCAGGC | 41 | 27 |
| 104189 | 5-10-5 MOE | 1142 | 1161 | GGCCCGGAGTCGACCTTCAC | 60 | 28 |
| 104190 | 5-10-5 MOE | 1207 | 1226 | GCGTTGGGATTGGTGGTGGC | 41 | 29 |
| 104191 | 5-10-5 MOE | 1275 | 1294 | AGTACACGCCACCATCACTG | 29 | 30 |
| 104192 | 5-10-5 MOE | 1343 | 1362 | TTTCCAGGGTTTGCCCAGCC | 80 | 31 |
| 104193 | 5-10-5 MOE | 1434 | 1453 | GGGCCTCCCAGGCTGGGTCC | 50 | 32 |
| 104194 | 5-10-5 MOE | 1537 | 1556 | CCCACAAACACCCCATGACG | 20 | 33 |
| 104195 | 5-10-5 MOE | 1581 | 1600 | CTTTGTGTTCTGCTGCAGCA | 55 | 34 |
| 104196 | 5-10-5 MOE | 1646 | 1665 | GTAGTGCCCGAAGTTGTAGC | 65 | 35 |
| 104197 | 5-10-5 MOE | 1726 | 1745 | TCACGCCGGAACCAGTTGAC | 56 | 36 |
| 104198 | 5-10-5 MOE | 1770 | 1789 | GAGCATTCTCCCCAAAGCCT | 72 | 37 |
| 104199 | 5-10-5 MOE | 1830 | 1849 | TGGGTGTCTCTCGGGCACTG | 43 | 38 |
| 104200 | 5-10-5 MOE | 1875 | 1894 | TGAGGCCGCTGAGATCCAAG | 57 | 39 |
| 104201 | 5-10-5 MOE | 1939 | 1958 | TCACGAACCTCCTGTTCCCA | 81 | 40 |
| 104202 | 5-10-5 MOE | 1981 | 2000 | GGCAGATCCTGGTTGACCTG | 53 | 41 |
| 104203 | 5-10-5 MOE | 2036 | 2055 | TCACATTTTGTGCACACGTC | 76 | 42 |
| 104205 | 5-10-5 MOE | 2094 | 2113 | TGCCTTCCCTATTCCCAGAT | 73 | 43 |
| 104206 | 5-10-5 MOE | 2136 | 2155 | AAGATGTTAGTTAATATCAA | 0 | 44 |
| 104207 | 5-10-5 MOE | 2170 | 2189 | GGACAGTCTTTGTGGGAAGG | 61 | 45 |

TABLE 3
Inhibition of human PEPCK-M RNA transcript in T24 cells
by antisense oligonucleotides targeting SEQ ID NO: 2

| | Target | Target | | | | SEQ |
|---|---|---|---|---|---|---|
| ISIS | Start | Stop | | | % | ID |
| No | Site | Site | Motif | Sequence | inhibition | NO. |
| 104129 | 3407 | 3426 | Uniform | AGGAACCGAGCGGAGCCGGG | 31 | 9 |
| 104130 | 3445 | 3464 | Uniform | TGCGGCCATGGCACCTGGGC | 44 | 10 |
| 104131 | 3455 | 3474 | Uniform | GGCGGTACAATGCGGCCATG | 18 | 11 |
| 104132 | 5971 | 5990 | Uniform | GCTACGGCATGATGGCCAGC | 0 | 12 |
| 104133 | 6025 | 6044 | Uniform | AATGCCAGTGGGAAGCTGGC | 0 | 13 |
| 104134 | 6088 | 6107 | Uniform | TCCATCACAGATGTGGATGC | 39 | 14 |
| 104135 | 6145 | 6164 | Uniform | TCGGATGAGGCCCTGCTGCT | 0 | 15 |
| 104136 | 7288 | 7307 | Uniform | CACCGTCTTGCTCTCTACTC | 58 | 16 |
| 104138 | 7417 | 7436 | Uniform | CTGCATGCAGCCTGGAAACC | 13 | 17 |
| 104139 | 7579 | 7598 | Uniform | GAGCTGCACCCCGATGCGGG | 0 | 18 |
| 104140 | 7628 | 7647 | Uniform | CCAGTCGGGTCATAATACGC | 24 | 19 |
| 104141 | 7674 | 7693 | Uniform | CACTTGACAAAGTCACCATC | 0 | 20 |
| 104142 | 8107 | 8126 | Uniform | TTGCACGGCCACTGGCTCAC | 4 | 21 |
| 104143 | 8154 | 8173 | Uniform | TGATCTCCCGCTGGTCGGGC | 24 | 22 |
| 104144 | 8198 | 8217 | Uniform | CTTGCCCAGCAGGGAGTTGC | 0 | 23 |
| 104145 | 8237 | 8256 | Uniform | CCGGGCCAGCCGAGAGGCGA | 0 | 24 |
| 104147 | 8651 | 8670 | Uniform | GGCACTAGGGAAGGCGGCTG | 8 | 26 |
| 104148 | 8700 | 8719 | Uniform | TCCAGCCTGGCAGTGCAGGC | 23 | 27 |
| 104150 | 9104 | 9123 | Uniform | GCGTTGGGATTGGTGGTGGC | 18 | 29 |
| 104151 | 9172 | 9191 | Uniform | AGTACACGCCACCATCACTG | 6 | 30 |
| 104152 | 9240 | 9259 | Uniform | TTTCCAGGGTTTGCCCAGCC | 43 | 31 |
| 104153 | 11870 | 11889 | Uniform | GGGCCTCCCAGGCTGGGTCC | 0 | 32 |
| 104154 | 12242 | 12261 | Uniform | CCCACAAACACCCCATGACG | 65 | 33 |
| 104155 | 12286 | 12305 | Uniform | CTTTGTGTTCTGCTGCAGCA | 50 | 34 |
| 104156 | 12605 | 12624 | Uniform | GTAGTGCCCGAAGTTGTAGC | 20 | 35 |
| 104157 | 12685 | 12704 | Uniform | TCACGCCGGAACCAGTTGAC | 0 | 36 |
| 104158 | 12729 | 12748 | Uniform | GAGCATTCTCCCCAAAGCCT | 2 | 37 |
| 104159 | 12789 | 12808 | Uniform | TGGGTGTCTCTCGGGCACTG | 10 | 38 |
| 104160 | 12834 | 12853 | Uniform | TGAGGCCGCTGAGATCCAAG | 34 | 39 |
| 104161 | 12898 | 12917 | Uniform | TCACGAACCTCCTGTTCCCA | 41 | 40 |
| 104162 | 12940 | 12959 | Uniform | GGCAGATCCTGGTTGACCTG | 18 | 41 |
| 104163 | 12995 | 13014 | Uniform | TCACATTTTGTGCACACGTC | 24 | 42 |
| 104165 | 13053 | 13072 | Uniform | TGCCTTCCCTATTCCCAGAT | 19 | 43 |
| 104166 | 13095 | 13114 | Uniform | AAGATGTTAGTTAATATCAA | 0 | 44 |
| 104167 | 13129 | 13148 | Uniform | GGACAGTCTTTGTGGGAAGG | 0 | 45 |
| 104169 | 3407 | 3426 | 5-10-5 MOE | AGGAACCGAGCGGAGCCGGG | 75 | 9 |
| 104170 | 3445 | 3464 | 5-10-5 MOE | TGCGGCCATGGCACCTGGGC | 57 | 10 |
| 104171 | 3455 | 3474 | 5-10-5 MOE | GGCGGTACAATGCGGCCATG | 55 | 11 |
| 104172 | 5971 | 5990 | 5-10-5 MOE | GCTACGGCATGATGGCCAGC | 47 | 12 |
| 104173 | 6025 | 6044 | 5-10-5 MOE | AATGCCAGTGGGAAGCTGGC | 3 | 13 |
| 104174 | 6088 | 6107 | 5-10-5 MOE | TCCATCACAGATGTGGATGC | 79 | 14 |
| 104175 | 6145 | 6164 | 5-10-5 MOE | TCGGATGAGGCCCTGCTGCT | 47 | 15 |
| 104176 | 7288 | 7307 | 5-10-5 MOE | CACCGTCTTGCTCTCTACTC | 85 | 16 |
| 104178 | 7417 | 7436 | 5-10-5 MOE | CTGCATGCAGCCTGGAAACC | 65 | 17 |
| 104179 | 7579 | 7598 | 5-10-5 MOE | GAGCTGCACCCCGATGCGGG | 48 | 18 |
| 104180 | 7628 | 7647 | 5-10-5 MOE | CCAGTCGGGTCATAATACGC | 81 | 19 |
| 104181 | 7674 | 7693 | 5-10-5 MOE | CACTTGACAAAGTCACCATC | 45 | 20 |
| 104182 | 8107 | 8126 | 5-10-5 MOE | TTGCACGGCCACTGGCTCAC | 70 | 21 |
| 104183 | 8154 | 8173 | 5-10-5 MOE | TGATCTCCCGCTGGTCGGGC | 78 | 22 |
| 104184 | 8198 | 8217 | 5-10-5 MOE | CTTGCCCAGCAGGGAGTTGC | 1 | 23 |
| 104185 | 8237 | 8256 | 5-10-5 MOE | CCGGGCCAGCCGAGAGGCGA | 33 | 24 |
| 104187 | 8651 | 8670 | 5-10-5 MOE | GGCACTAGGGAAGGCGGCTG | 62 | 26 |
| 104188 | 8700 | 8719 | 5-10-5 MOE | TCCAGCCTGGCAGTGCAGGC | 41 | 27 |
| 104190 | 9104 | 9123 | 5-10-5 MOE | GCGTTGGGATTGGTGGTGGC | 41 | 29 |
| 104191 | 9172 | 9191 | 5-10-5 MOE | AGTACACGCCACCATCACTG | 29 | 30 |
| 104192 | 9240 | 9259 | 5-10-5 MOE | TTTCCAGGGTTTGCCCAGCC | 80 | 31 |
| 104193 | 11870 | 11889 | 5-10-5 MOE | GGGCCTCCCAGGCTGGGTCC | 50 | 32 |
| 104194 | 12242 | 12261 | 5-10-5 MOE | CCCACAAACACCCCATGACG | 20 | 33 |
| 104195 | 12286 | 12305 | 5-10-5 MOE | CTTTGTGTTCTGCTGCAGCA | 55 | 34 |
| 104196 | 12605 | 12624 | 5-10-5 MOE | GTAGTGCCCGAAGTTGTAGC | 65 | 35 |
| 104197 | 12685 | 12704 | 5-10-5 MOE | TCACGCCGGAACCAGTTGAC | 56 | 36 |
| 104198 | 12729 | 12748 | 5-10-5 MOE | GAGCATTCTCCCCAAAGCCT | 72 | 37 |
| 104199 | 12789 | 12808 | 5-10-5 MOE | TGGGTGTCTCTCGGGCACTG | 43 | 38 |
| 104200 | 12834 | 12853 | 5-10-5 MOE | TGAGGCCGCTGAGATCCAAG | 57 | 39 |
| 104201 | 12898 | 12917 | 5-10-5 MOE | TCACGAACCTCCTGTTCCCA | 81 | 40 |
| 104202 | 12940 | 12959 | 5-10-5 MOE | GGCAGATCCTGGTTGACCTG | 53 | 41 |
| 104203 | 12995 | 13014 | 5-10-5 MOE | TCACATTTTGTGCACACGTC | 76 | 42 |
| 104205 | 13053 | 13072 | 5-10-5 MOE | TGCCTTCCCTATTCCCAGAT | 73 | 43 |
| 104206 | 13095 | 13114 | 5-10-5 MOE | AAGATGTTAGTTAATATCAA | 0 | 44 |
| 104207 | 13129 | 13148 | 5-10-5 MOE | GGACAGTCTTTGTGGGAAGG | 61 | 45 |

TABLE 4
Inhibition of human PEPCK-M RNA transcript in T24 cells
by antisense oligonucleotides targeting SEQ ID NO: 3

| | | Target | Target | | | SEQ |
|---|---|---|---|---|---|---|
| ISIS | | Start | Stop | | % | ID |
| No | Motif | Site | Site | Sequence | inhibition | NO. |
| 104129 | Uniform | 18 | 37 | AGGAACCGAGCGGAGCCGGG | 31 | 9 |
| 104130 | Uniform | 56 | 75 | TGCGGCCATGGCACCTGGGC | 44 | 10 |
| 104131 | Uniform | 66 | 85 | GGCGGTACAATGCGGCCATG | 18 | 11 |
| 104132 | Uniform | 125 | 144 | GCTACGGCATGATGGCCAGC | 0 | 12 |
| 104133 | Uniform | 179 | 198 | AATGCCAGTGGGAAGCTGGC | 0 | 13 |
| 104134 | Uniform | 242 | 261 | TCCATCACAGATGTGGATGC | 39 | 14 |
| 104135 | Uniform | 299 | 318 | TCGGATGAGGCCCTGCTGCT | 0 | 15 |
| 104136 | Uniform | 377 | 396 | CACCGTCTTGCTCTCTACTC | 58 | 16 |
| 104137 | Uniform | 422 | 441 | ACCAGGCGGGAGTGGTACCG | 33 | 46 |
| 104138 | Uniform | 506 | 525 | CTGCATGCAGCCTGGAAACC | 13 | 17 |
| 104139 | Uniform | 581 | 600 | GAGCTGCACCCCGATGCGGG | 0 | 18 |
| 104140 | Uniform | 630 | 649 | CCAGTCGGGTCATAATACGC | 24 | 19 |
| 104141 | Uniform | 676 | 695 | CACTTGACAAAGTCACCATC | 0 | 20 |
| 104142 | Uniform | 739 | 758 | TTGCACGGCCACTGGCTCAC | 4 | 21 |
| 104143 | Uniform | 786 | 805 | TGATCTCCCGCTGGTCGGGC | 24 | 22 |
| 104144 | Uniform | 830 | 849 | CTTGCCCAGCAGGGAGTTGC | 0 | 23 |
| 104145 | Uniform | 869 | 888 | CCGGGCCAGCCGAGAGGCGA | 0 | 24 |
| 104146 | Uniform | 914 | 933 | GGTGATGCCCAGGATCAGCA | 21 | 25 |
| 104147 | Uniform | 962 | 981 | GGCACTAGGGAAGGCGGCTG | 8 | 26 |
| 104148 | Uniform | 1011 | 1030 | TCCAGCCTGGCAGTGCAGGC | 23 | 27 |
| 104149 | Uniform | 1076 | 1095 | GGCCCGGAGTCGACCTTCAC | 32 | 28 |
| 104150 | Uniform | 1141 | 1160 | GCGTTGGGATTGGTGGTGGC | 18 | 29 |
| 104151 | Uniform | 1209 | 1228 | AGTACACGCCACCATCACTG | 6 | 30 |
| 104152 | Uniform | 1277 | 1296 | TTTCCAGGGTTTGCCCAGCC | 43 | 31 |
| 104153 | Uniform | 1368 | 1387 | GGGCCTCCCAGGCTGGGTCC | 0 | 32 |
| 104154 | Uniform | 1471 | 1490 | CCCACAAACACCCCATGACG | 65 | 33 |
| 104155 | Uniform | 1515 | 1534 | CTTTGTGTTCTGCTGCAGCA | 50 | 34 |
| 104156 | Uniform | 1580 | 1599 | GTAGTGCCCGAAGTTGTAGC | 20 | 35 |
| 104157 | Uniform | 1660 | 1679 | TCACGCCGGAACCAGTTGAC | 0 | 36 |
| 104158 | Uniform | 1704 | 1723 | GAGCATTCTCCCCAAAGCCT | 2 | 37 |
| 104159 | Uniform | 1764 | 1783 | TGGGTGTCTCTCGGGCACTG | 10 | 38 |
| 104160 | Uniform | 1809 | 1828 | TGAGGCCGCTGAGATCCAAG | 34 | 39 |
| 104161 | Uniform | 1873 | 1892 | TCACGAACCTCCTGTTCCCA | 41 | 40 |
| 104162 | Uniform | 1915 | 1934 | GGCAGATCCTGGTTGACCTG | 18 | 41 |
| 104163 | Uniform | 1970 | 1989 | TCACATTTTGTGCACACGTC | 24 | 42 |
| 104164 | Uniform | 1985 | 2004 | AGACTAGGCCTCAGGTCACA | 10 | 47 |
| 104165 | Uniform | 2027 | 2046 | TGCCTTCCCTATTCCCAGAT | 19 | 43 |
| 104166 | Uniform | 2068 | 2087 | AAGATGTTAGTTAATATCAA | 0 | 44 |
| 104167 | Uniform | 2102 | 2121 | GGACAGTCTTTGTGGGAAGG | 0 | 45 |
| 104168 | Uniform | 2130 | 2149 | TTAAAATAGATAAGCATCTC | 0 | 48 |
| 104169 | 5-10-5 MOE | 18 | 37 | AGGAACCGAGCGGAGCCGGG | 75 | 9 |
| 104170 | 5-10-5 MOE | 56 | 75 | TGCGGCCATGGCACCTGGGC | 57 | 10 |
| 104171 | 5-10-5 MOE | 66 | 85 | GGCGGTACAATGCGGCCATG | 55 | 11 |
| 104172 | 5-10-5 MOE | 125 | 144 | GCTACGGCATGATGGCCAGC | 47 | 12 |
| 104173 | 5-10-5 MOE | 179 | 198 | AATGCCAGTGGGAAGCTGGC | 3 | 13 |
| 104174 | 5-10-5 MOE | 242 | 261 | TCCATCACAGATGTGGATGC | 79 | 14 |
| 104175 | 5-10-5 MOE | 299 | 318 | TCGGATGAGGCCCTGCTGCT | 47 | 15 |
| 104176 | 5-10-5 MOE | 377 | 396 | CACCGTCTTGCTCTCTACTC | 85 | 16 |
| 104177 | 5-10-5 MOE | 422 | 441 | ACCAGGCGGGAGTGGTACCG | 56 | 46 |
| 104178 | 5-10-5 MOE | 506 | 525 | CTGCATGCAGCCTGGAAACC | 65 | 17 |
| 104179 | 5-10-5 MOE | 581 | 600 | GAGCTGCACCCCGATGCGGG | 48 | 18 |
| 104180 | 5-10-5 MOE | 630 | 649 | CCAGTCGGGTCATAATACGC | 81 | 19 |
| 104181 | 5-10-5 MOE | 676 | 695 | CACTTGACAAAGTCACCATC | 45 | 20 |
| 104182 | 5-10-5 MOE | 739 | 758 | TTGCACGGCCACTGGCTCAC | 70 | 21 |
| 104183 | 5-10-5 MOE | 786 | 805 | TGATCTCCCGCTGGTCGGGC | 78 | 22 |
| 104184 | 5-10-5 MOE | 830 | 849 | CTTGCCCAGCAGGGAGTTGC | 1 | 23 |
| 104185 | 5-10-5 MOE | 869 | 888 | CCGGGCCAGCCGAGAGGCGA | 33 | 24 |
| 104186 | 5-10-5 MOE | 914 | 933 | GGTGATGCCCAGGATCAGCA | 18 | 25 |
| 104187 | 5-10-5 MOE | 962 | 981 | GGCACTAGGGAAGGCGGCTG | 62 | 26 |
| 104188 | 5-10-5 MOE | 1011 | 1030 | TCCAGCCTGGCAGTGCAGGC | 41 | 27 |
| 104189 | 5-10-5 MOE | 1076 | 1095 | GGCCCGGAGTCGACCTTCAC | 60 | 28 |
| 104190 | 5-10-5 MOE | 1141 | 1160 | GCGTTGGGATTGGTGGTGGC | 41 | 29 |
| 104191 | 5-10-5 MOE | 1209 | 1228 | AGTACACGCCACCATCACTG | 29 | 30 |
| 104192 | 5-10-5 MOE | 1277 | 1296 | TTTCCAGGGTTTGCCCAGCC | 80 | 31 |
| 104193 | 5-10-5 MOE | 1368 | 1387 | GGGCCTCCCAGGCTGGGTCC | 50 | 32 |
| 104194 | 5-10-5 MOE | 1471 | 1490 | CCCACAAACACCCCATGACG | 20 | 33 |
| 104195 | 5-10-5 MOE | 1515 | 1534 | CTTTGTGTTCTGCTGCAGCA | 55 | 34 |
| 104196 | 5-10-5 MOE | 1580 | 1599 | GTAGTGCCCGAAGTTGTAGC | 65 | 35 |
| 104197 | 5-10-5 MOE | 1660 | 1679 | TCACGCCGGAACCAGTTGAC | 56 | 36 |
| 104198 | 5-10-5 MOE | 1704 | 1723 | GAGCATTCTCCCCAAAGCCT | 72 | 37 |
| 104199 | 5-10-5 MOE | 1764 | 1783 | TGGGTGTCTCTCGGGCACTG | 43 | 38 |
| 104200 | 5-10-5 MOE | 1809 | 1828 | TGAGGCCGCTGAGATCCAAG | 57 | 39 |
| 104201 | 5-10-5 MOE | 1873 | 1892 | TCACGAACCTCCTGTTCCCA | 81 | 40 |
| 104202 | 5-10-5 MOE | 1915 | 1934 | GGCAGATCCTGGTTGACCTG | 53 | 41 |
| 104203 | 5-10-5 MOE | 1970 | 1989 | TCACATTTTGTGCACACGTC | 76 | 42 |
| 104204 | 5-10-5 MOE | 1985 | 2004 | AGACTAGGCCTCAGGTCACA | 13 | 47 |
| 104205 | 5-10-5 MOE | 2027 | 2046 | TGCCTTCCCTATTCCCAGAT | 73 | 43 |
| 104206 | 5-10-5 MOE | 2068 | 2087 | AAGATGTTAGTTAATATCAA | 0 | 44 |
| 104207 | 5-10-5 MOE | 2102 | 2121 | GGACAGTCTTTGTGGGAAGG | 61 | 45 |
| 104208 | 5-10-5 MOE | 2130 | 2149 | TTAAAATAGATAAGCATCTC | 36 | 48 |

### Example 2

### Antisense Inhibition of Rat PEPCK-M in Primary Rat Hepatocytes

Antisense oligonucleotides targeted to a rat PEPCK-M nucleic acid were tested for their effect on PEPCK-M RNA transcript in vitro. Primary rat hepatocytes were cultured at a density of 20,000 cells per well were transfected using Cytofectin reagent with 100 nM antisense oligonucleotide. After approximately 24 hours, RNA was isolated from the cells and PEPCK-M RNA transcript levels were measured by quantitative real-time PCR with rat primer probe set RTS3036 (forward sequence TGGGAAAGCCATGGAAACC, designated herein as SEQ ID NO: 49; reverse sequence GCGAGCCGGGACACAA, designated herein as SEQ ID NO: 50; probe sequence ACAAGGAACCCTGTGCGCATCCAAX, designated herein as SEQ ID NO: 51). PEPCK-M RNA transcript levels were adjusted according to total RNA content, as measured by RIBOGREEN®. Results are presented as percent inhibition of PEPCK-M, relative to untreated control cells.

The antisense oligonucleotides in Tables 5 and 6 are 5-10-5 gapmers where the gap segment comprises ten 2′-deoxynucleosides and each wing segment comprises five 2′-MOE nucleosides. The internucleoside linkages throughout each gapmer are phosphorothioate (P═S) linkages. All cytidine residues throughout each gapmer are 5-methylcytidines. ‘Rat Target start site’ indicates the 5′-most nucleotide to which the antisense oligonucleotide is targeted. ‘Rat Target stop site’ indicates the 3′-most nucleotide to which the antisense oligonucleotide is targeted. All the antisense oligonucleotides listed in Table 5 target SEQ ID NO: 4 (GENBANK Accession No. XM_{—}001055522.1). All the antisense oligonucleotides listed in Table 6 target SEQ ID NO: 5 (GENBANK Accession No. NW_{—}047454.2 truncated from nucleotides 5520000 to 5546000).

The rat oligonucleotides of Tables 5 and 6 may also be cross-reactive with human gene sequences. ‘Mismatches’ indicate the number of nucleobases by which the rat oligonucleotide is mismatched with a human gene sequence. The greater the complementarity between the rat oligonucleotide and the human sequence, the more likely the rat oligonucleotide can cross-react with the human sequence. The rat oligonucleotides in Tables 5 and 6 were compared to SEQ ID NO: 1 (GENBANK Accession No. NM_{—}004563.2). “Human Target start site” indicates the 5′-most nucleotide to which the gapmer is targeted in the human gene sequence.

TABLE 5
Inhibition of rat PEPCK-M RNA transcript in primary rat hepatocytes
by antisense oligonucleotides targeting SEQ ID NO: 4

| Rat | Rat | | | | | Human | |
|---|---|---|---|---|---|---|---|
| Target | Target | | | | SEQ | Target | |
| Start | Stop | | | % | ID | Start | Mis- |
| Site | Site | ISIS No | Sequence | inhibition | NO | Site | matches |
| 140 | 159 | 421005 | CACTAGCCCGGGCTCAAGGC | 23 | 52 | n/a | n/a |
| 151 | 170 | 421006 | GTAGGCCGGCTCACTAGCCC | 28 | 53 | n/a | n/a |
| 183 | 202 | 421007 | GGGCGGAACCTAGCTGGTTC | 0 | 54 | n/a | n/a |
| 311 | 330 | 421008 | GCTGCGGGATGGCCACAGGA | 68 | 55 | n/a | n/a |
| 333 | 352 | 421009 | CAGCCATGGCACCTGGACTG | 69 | 56 | 120 | 3 |
| 345 | 364 | 421010 | GGAGGTACATAGCAGCCATG | 53 | 57 | n/a | n/a |
| 387 | 406 | 421011 | GGCACCAGGGCCTCAGCCTG | 48 | 58 | n/a | n/a |
| 403 | 422 | 421012 | CTACGGCATGGTGACCGGCA | 62 | 59 | n/a | n/a |
| 617 | 636 | 421013 | TGTGCGGGCCAGCCAGCAGT | 62 | 60 | 404 | 0 |
| 640 | 659 | 421014 | ACCCGTGCCACATCCTTGGG | 72 | 61 | 427 | 1 |
| 702 | 721 | 421015 | CACCAGCCAGGAGAGGCACT | 81 | 62 | n/a | n/a |
| 709 | 728 | 421016 | CTGGCCCCACCAGCCAGGAG | 46 | 63 | 496 | 3 |
| 730 | 749 | 421017 | ATCCAGTTGCCCAGCTGCCC | 87 | 64 | 517 | 0 |
| 787 | 806 | 421018 | CCCTGCATGCATCCTGGGAA | 65 | 65 | 574 | 2 |
| 824 | 843 | 421019 | GGGACCCATGCTGAACGGAA | 80 | 66 | 611 | 2 |
| 833 | 852 | 421020 | GGAGCCCAAGGGACCCATGC | 71 | 67 | 620 | 2 |
| 874 | 893 | 421021 | TAAGGCGAGTCAGTGAGCTG | 66 | 68 | 661 | 3 |
| 914 | 933 | 421022 | TGTCCCCAGGCGGGTCATAA | 60 | 69 | 701 | 1 |
| 927 | 946 | 421023 | CCTGGAGTACATGTGTCCCC | 63 | 70 | 714 | 3 |
| 976 | 995 | 421024 | GGCTGGCCCACCGAATGCAG | 68 | 71 | 763 | 2 |
| 999 | 1018 | 421025 | CAGGATCCCCATGTCCAGTC | 81 | 72 | n/a | n/a |
| 1012 | 1031 | 421026 | GGCCACCGGCCCACAGGATC | 68 | 73 | n/a | n/a |
| 1019 | 1038 | 421027 | ATTGCATGGCCACCGGCCCA | 57 | 74 | n/a | n/a |
| 1025 | 1044 | 421028 | TTCCGGATTGCATGGCCACC | 69 | 75 | n/a | n/a |
| 1044 | 1063 | 421029 | CGTGGCCAATCAGGGTTTTT | 71 | 76 | 831 | 1 |
| 1107 | 1126 | 421030 | TGCCCAGCAAGGAGTTCCCA | 72 | 77 | 894 | 2 |
| 1136 | 1155 | 421031 | AGAGGCGATGCGCAGGGCAA | 63 | 78 | 923 | 1 |
| 1149 | 1168 | 421032 | CCCTGGCCAGGCGAGAGGCG | 68 | 79 | 936 | 2 |
| 1158 | 1177 | 421033 | AGCCCTCATCCCTGGCCAGG | 73 | 80 | 945 | 2 |
| 1197 | 1216 | 421034 | GGTTGGTGATGCCCAAAATC | 78 | 81 | 984 | 3 |
| 1271 | 1290 | 421035 | CCGCATCATGGCCAGATTGG | 74 | 82 | 1058 | 2 |
| 1354 | 1373 | 421036 | GCCCGGAGTTGACCTTCACT | 80 | 83 | 1141 | 1 |
| 1368 | 1387 | 421037 | TCTCAGGGTTGATGGCCCGG | 63 | 84 | 1155 | 0 |
| 1376 | 1395 | 421038 | GAAGCCATTCTCAGGGTTGA | 73 | 85 | 1163 | 1 |
| 1410 | 1429 | 421039 | TGGTGGTGGCAGAGGTACCA | 73 | 86 | 1197 | 0 |
| 1427 | 1446 | 421040 | GGCCATGGCATTGGGATTGG | 63 | 87 | 1214 | 2 |
| 1513 | 1532 | 421041 | GGAAGAGGCTGGTCAATGCC | 53 | 88 | 1300 | 0 |
| 1520 | 1539 | 421042 | ACCAGGTGGAAGAGGCTGGT | 64 | 89 | 1307 | 0 |
| 1562 | 1581 | 421043 | CCCAGGTTTCCATGGCTTTC | 92 | 90 | n/a | n/a |
| 1620 | 1639 | 421044 | GGCACTGGCGAGCCGGGACA | 91 | 91 | 1407 | 1 |
| 1662 | 1681 | 421045 | TTGGAACACCTTCTGGTGCC | 74 | 92 | n/a | n/a |
| 1706 | 1725 | 421046 | TGGTACCCCTTTAGGTCTGC | 81 | 93 | 1493 | 2 |
| 1714 | 1733 | 421047 | TACACCAGTGGTACCCCTTT | 60 | 94 | 1501 | 2 |
| 1774 | 1793 | 421048 | GTGGACTCAGAGCGCATGGC | 79 | 95 | 1561 | 0 |
| 1911 | 1930 | 421049 | TACGAGGCAGCCGGGCACCT | 90 | 96 | n/a | n/a |
| 1962 | 1981 | 421050 | GCCACAGGAAGCGGCCTGCT | 72 | 97 | 1749 | 3 |
| 1971 | 1990 | 421051 | CAAAGCCTGGCCACAGGAAG | 64 | 98 | 1758 | 0 |
| 2005 | 2024 | 421052 | CGGCAGATCCAGTCTAGCAC | 66 | 99 | 1792 | 0 |
| 2062 | 2081 | 421053 | TCCTTTGGTACGAGCCCAAT | 65 | 100 | 1849 | 2 |
| 2086 | 2105 | 421054 | AGGCCACTGAGATCCAGGGC | 69 | 101 | 1873 | 2 |
| 2093 | 2112 | 421055 | TGCTCGGAGGCCACTGAGAT | 88 | 102 | 1880 | 3 |
| 2103 | 2122 | 421056 | TGGTATCTATTGCTCGGAGG | 58 | 103 | 1890 | 3 |
| 2121 | 2140 | 421057 | GGATGGAGAACAGCTGACTG | 72 | 104 | 1908 | 2 |
| 2140 | 2159 | 421058 | TGTTCCCAGAAGTCCTTGGG | 86 | 105 | 1927 | 0 |
| 2197 | 2216 | 421059 | TTGGGCAGATCCTGGTTGAC | 70 | 106 | 1984 | 0 |
| 2218 | 2237 | 421060 | TCGAGCTCAGCCAACACCTC | 69 | 107 | 2005 | 1 |
| 2225 | 2244 | 421061 | CAGGGCCTCGAGCTCAGCCA | 57 | 108 | 2012 | 1 |
| 2238 | 2257 | 421062 | GCACGCGCTCTTCCAGGGCC | 92 | 109 | n/a | n/a |
| 2266 | 2285 | 421063 | TAGCCTAAGGCCTCAGGTCA | 76 | 110 | 2053 | 2 |
| 2302 | 2321 | 421064 | GGATCCTCTACCCAGATGGG | 73 | 111 | n/a | n/a |
| 2453 | 2472 | 421065 | GAAGCCCGTGAGGACAAATG | 62 | 112 | n/a | n/a |
| 2477 | 2496 | 421066 | GCCAGCATCGAGACTGACAA | 56 | 113 | n/a | n/a |
| 2507 | 2526 | 421067 | TGCCTTCTTCCAGAAGTTCC | 39 | 114 | n/a | n/a |
| 2612 | 2631 | 421068 | ATATAGACCAGGCAGGCTGG | 50 | 115 | n/a | n/a |
| 2840 | 2859 | 421069 | GGCAACTGGGAGGAAACCAG | 55 | 116 | n/a | n/a |
| 3117 | 3136 | 421070 | GGGTCCTTCCAAGTGCTAGG | 56 | 117 | n/a | n/a |
| 3130 | 3149 | 421071 | TGCTTTGTGATTAGGGTCCT | 71 | 118 | n/a | n/a |

TABLE 6
Inhibition of rat PEPCK-M RNA transcript in primary rat hepatocytes
by antisense oligonucleotides targeting SEQ ID NO: 5

| Rat | Rat | | | | | Human | |
|---|---|---|---|---|---|---|---|
| Target | Target | | | | SEQ | Target | |
| Start | Stop | | | % | ID | Start | Mis- |
| Site | Site | ISIS No | Sequence | inhibition | NO. | Site | matches |
| 13694 | 13713 | 421005 | CACTAGCCCGGGCTCAAGGC | 23 | 52 | n/a | n/a |
| 13705 | 13724 | 421006 | GTAGGCCGGCTCACTAGCCC | 28 | 53 | n/a | n/a |
| 13737 | 13756 | 421007 | GGGCGGAACCTAGCTGGTTC | 0 | 54 | n/a | n/a |
| 13865 | 13884 | 421008 | GCTGCGGGATGGCCACAGGA | 68 | 55 | n/a | n/a |
| 13887 | 13906 | 421009 | CAGCCATGGCACCTGGACTG | 69 | 56 | 120 | 3 |
| 13899 | 13918 | 421010 | GGAGGTACATAGCAGCCATG | 53 | 57 | n/a | n/a |
| 15833 | 15852 | 421011 | GGCACCAGGGCCTCAGCCTG | 48 | 58 | n/a | n/a |
| 15849 | 15868 | 421012 | CTACGGCATGGTGACCGGCA | 62 | 59 | n/a | n/a |
| 16792 | 16811 | 421014 | ACCCGTGCCACATCCTTGGG | 72 | 60 | 427 | 1 |
| 16854 | 16873 | 421015 | CACCAGCCAGGAGAGGCACT | 81 | 61 | n/a | n/a |
| 16861 | 16880 | 421016 | CTGGCCCCACCAGCCAGGAG | 46 | 62 | 496 | 3 |
| 16882 | 16901 | 421017 | ATCCAGTTGCCCAGCTGCCC | 87 | 63 | 517 | 0 |
| 16939 | 16958 | 421018 | CCCTGCATGCATCCTGGGAA | 65 | 64 | 574 | 2 |
| 17062 | 17081 | 421019 | GGGACCCATGCTGAACGGAA | 80 | 65 | 611 | 2 |
| 17071 | 17090 | 421020 | GGAGCCCAAGGGACCCATGC | 71 | 66 | 620 | 2 |
| 17112 | 17131 | 421021 | TAAGGCGAGTCAGTGAGCTG | 66 | 67 | 661 | 3 |
| 17152 | 17171 | 421022 | TGTCCCCAGGCGGGTCATAA | 60 | 68 | 701 | 1 |
| 17165 | 17184 | 421023 | CCTGGAGTACATGTGTCCCC | 63 | 69 | 714 | 3 |
| 17214 | 17233 | 421024 | GGCTGGCCCACCGAATGCAG | 68 | 70 | 763 | 2 |
| 17577 | 17596 | 421026 | GGCCACCGGCCCACAGGATC | 68 | 71 | n/a | n/a |
| 17584 | 17603 | 421027 | ATTGCATGGCCACCGGCCCA | 57 | 72 | n/a | n/a |
| 17590 | 17609 | 421028 | TTCCGGATTGCATGGCCACC | 69 | 73 | n/a | n/a |
| 17609 | 17628 | 421029 | CGTGGCCAATCAGGGTTTTT | 71 | 74 | 831 | 1 |
| 17672 | 17691 | 421030 | TGCCCAGCAAGGAGTTCCCA | 72 | 75 | 894 | 2 |
| 17701 | 17720 | 421031 | AGAGGCGATGCGCAGGGCAA | 63 | 76 | 923 | 1 |
| 17714 | 17733 | 421032 | CCCTGGCCAGGCGAGAGGCG | 68 | 77 | 936 | 2 |
| 17723 | 17742 | 421033 | AGCCCTCATCCCTGGCCAGG | 73 | 78 | 945 | 2 |
| 18070 | 18089 | 421034 | GGTTGGTGATGCCCAAAATC | 78 | 79 | 984 | 3 |
| 18144 | 18163 | 421035 | CCGCATCATGGCCAGATTGG | 74 | 80 | 1058 | 2 |
| 18397 | 18416 | 421037 | TCTCAGGGTTGATGGCCCGG | 63 | 81 | 1155 | 0 |
| 18405 | 18424 | 421038 | GAAGCCATTCTCAGGGTTGA | 73 | 82 | 1163 | 1 |
| 18439 | 18458 | 421039 | TGGTGGTGGCAGAGGTACCA | 73 | 86 | 1197 | 0 |
| 18456 | 18475 | 421040 | GGCCATGGCATTGGGATTGG | 63 | 87 | 1214 | 2 |
| 18542 | 18561 | 421041 | GGAAGAGGCTGGTCAATGCC | 53 | 88 | 1300 | 0 |
| 18549 | 18568 | 421042 | ACCAGGTGGAAGAGGCTGGT | 64 | 89 | 1307 | 0 |
| 20658 | 20677 | 421044 | GGCACTGGCGAGCCGGGACA | 91 | 91 | 1407 | 1 |
| 20700 | 20719 | 421045 | TTGGAACACCTTCTGGTGCC | 74 | 92 | n/a | n/a |
| 21032 | 21051 | 421048 | GTGGACTCAGAGCGCATGGC | 79 | 95 | 1561 | 0 |
| 22060 | 22079 | 421049 | TACGAGGCAGCCGGGCACCT | 90 | 96 | n/a | n/a |
| 22111 | 22130 | 421050 | GCCACAGGAAGCGGCCTGCT | 72 | 97 | 1749 | 3 |
| 22120 | 22139 | 421051 | CAAAGCCTGGCCACAGGAAG | 64 | 98 | 1758 | 0 |
| 22154 | 22173 | 421052 | CGGCAGATCCAGTCTAGCAC | 66 | 99 | 1792 | 0 |
| 22211 | 22230 | 421053 | TCCTTTGGTACGAGCCCAAT | 65 | 100 | 1849 | 2 |
| 22235 | 22254 | 421054 | AGGCCACTGAGATCCAGGGC | 69 | 101 | 1873 | 2 |
| 22242 | 22261 | 421055 | TGCTCGGAGGCCACTGAGAT | 88 | 102 | 1880 | 3 |
| 22252 | 22271 | 421056 | TGGTATCTATTGCTCGGAGG | 58 | 103 | 1890 | 3 |
| 22270 | 22289 | 421057 | GGATGGAGAACAGCTGACTG | 72 | 104 | 1908 | 2 |
| 22289 | 22308 | 421058 | TGTTCCCAGAAGTCCTTGGG | 86 | 105 | 1927 | 0 |
| 22346 | 22365 | 421059 | TTGGGCAGATCCTGGTTGAC | 70 | 106 | 1984 | 0 |
| 22367 | 22386 | 421060 | TCGAGCTCAGCCAACACCTC | 69 | 107 | 2005 | 1 |
| 22374 | 22393 | 421061 | CAGGGCCTCGAGCTCAGCCA | 57 | 108 | 2012 | 1 |
| 22387 | 22406 | 421062 | GCACGCGCTCTTCCAGGGCC | 92 | 109 | n/a | n/a |
| 22415 | 22434 | 421063 | TAGCCTAAGGCCTCAGGTCA | 76 | 110 | 2053 | 2 |
| 22451 | 22470 | 421064 | GGATCCTCTACCCAGATGGG | 73 | 111 | n/a | n/a |
| 22602 | 22621 | 421065 | GAAGCCCGTGAGGACAAATG | 62 | 112 | n/a | n/a |
| 22626 | 22645 | 421066 | GCCAGCATCGAGACTGACAA | 56 | 113 | n/a | n/a |
| 22656 | 22675 | 421067 | TGCCTTCTTCCAGAAGTTCC | 39 | 114 | n/a | n/a |
| 22761 | 22780 | 421068 | ATATAGACCAGGCAGGCTGG | 50 | 115 | n/a | n/a |
| 22989 | 23008 | 421069 | GGCAACTGGGAGGAAACCAG | 55 | 116 | n/a | n/a |
| 23266 | 23285 | 421070 | GGGTCCTTCCAAGTGCTAGG | 56 | 117 | n/a | n/a |
| 23279 | 23298 | 421071 | TGCTTTGTGATTAGGGTCCT | 71 | 118 | n/a | n/a |
| 23610 | 23629 | 421072 | GCCCAGTGTGGCTGCTGAAC | 56 | 119 | n/a | n/a |
| 23873 | 23892 | 421073 | GCTCACTGCCCCAGAGTGGG | 46 | 120 | n/a | n/a |
| 23891 | 23910 | 421074 | TGTGTGCCACCATGCTCAGC | 54 | 121 | n/a | n/a |
| 9007 | 9026 | 421075 | AGCCTGCGCCGCCAGCTGGC | 27 | 122 | n/a | n/a |
| 13918 | 13937 | 421076 | GTCACTCACCGCAGGCCGGG | 49 | 123 | n/a | n/a |
| 16948 | 16967 | 421077 | GACTTGTTACCCTGCATGCA | 53 | 124 | n/a | n/a |
| 17036 | 17055 | 421078 | ACATGGTGCGGCCTGCGGAG | 52 | 125 | n/a | n/a |
| 17237 | 17256 | 421079 | GGTACTTACCATGTCCAGTC | 45 | 126 | n/a | n/a |
| 17567 | 17586 | 421080 | CCACAGGATCCCCTGGAGAC | 76 | 127 | n/a | n/a |
| 18601 | 18620 | 421081 | GGGACCACATACCAGGTTTC | 46 | 128 | n/a | n/a |
| 20965 | 20984 | 421082 | GTGGTACCCCTGGAACACCA | 55 | 129 | n/a | n/a |

### Example 3

### Dose-Dependent Antisense Inhibition of Rat PEPCK-M in Rat Primary Hepatocytes

Antisense oligonucleotides exhibiting inhibition of PEPCK-M in rat primary hepatocytes (see Example 2) were tested at various doses. Cells were plated at a density of 20,000 cells per well and transfected using Cytofectin® reagent with 12.5 nM, 25 nM, 50 nM, 100 nM, and 200 nM concentrations of each antisense oligonucleotide. After approximately 16 hours, RNA was isolated from the cells and PEPCK-M transcript levels were measured by quantitative real-time PCR using primer probe set RTS3036. PEPCK-M transcript levels were normalized to total RNA content, as measured by RIBOGREEN®. Results are presented in Table 7 as percent inhibition of PEPCK-M, relative to untreated control cells.

TABLE 7
Dose-dependent antisense inhibition of rat
PEPCK-M in rat primary hepatocytes

| ISIS | 12.5 | 25.0 | 50.0 | 100.0 | 200.0 | IC₅₀ |
|---|---|---|---|---|---|---|
| No. | nM | nM | nM | nM | nM | (nM) |
| 421015 | 0 | 23 | 53 | 76 | 92 | 85.4 |
| 421017 | 13 | 21 | 42 | 67 | 89 | 71.6 |
| 421025 | 8 | 15 | 39 | 68 | 85 | 79.4 |
| 421034 | 0 | 15 | 36 | 62 | 82 | 70.2 |
| 421036 | 14 | 23 | 39 | 69 | 87 | 72.0 |
| 421046 | 11 | 10 | 33 | 51 | 78 | 54.1 |
| 421048 | 15 | 32 | 43 | 64 | 85 | 60.2 |
| 421049 | 8 | 10 | 18 | 54 | 91 | 56.9 |
| 421055 | 4 | 7 | 36 | 61 | 85 | 63.4 |
| 421058 | 11 | 17 | 35 | 67 | 92 | 57.0 |
| 421062 | 0 | 14 | 30 | 67 | 87 | 52.7 |
| 421063 | 12 | 12 | 47 | 60 | 84 | 64.5 |

### Example 4

### In Vivo Antisense Inhibition of Rat PEPCK-M

Metabolic endpoints of ISIS oligonucleotides targeting PEPCK-M were evaluated in Sprague-Dawley rats.

ISIS 421062, which demonstrated statistically significant dose-dependent inhibition in vitro (see Example 3), was selected for further evaluation in vivo.

### Treatment

Sprague-Dawley rats were maintained on a 12-hour light/dark cycle and fed ad libitum normal chow Animals were acclimated for at least 7 days in the research facility before initiation of the experiment. Antisense oligonucleotides were prepared in PBS and sterilized by filtering through a 0.2 micron filter. Oligonucleotides were dissolved in 0.9% PBS for injection.

The rats were divided into two treatment groups of nine weight-matched rats each. The first group was injected intraperitoneally with ISIS 421062 at a dose of 50 mg/kg/week for 8 doses. The second group was injected intraperitoneally with control oligonucleotide ISIS 141923 (CCTTCCCTGAAGGTTCCTCC, 5-10-5 MOE gapmer with no known rat target sequence (SEQ ID NO: 130)) at a dose of 50 mg/kg/week for 8 doses. The control oligonucleotide-dosed group served as the control to which the first group was compared. The rats were weighed once a week.

Inhibition of PEPCK-M mRNA

Twenty four hours after the final dose, the animals were sacrificed and livers were harvested. RNA was isolated for real-time PCR analysis of PEPCK-M. Treatment with ISIS 421062 reduced rat PEPCK-M RNA by 77% compared to the control group.

### Effect on Fasted and Fed Glucose and Insulin Levels

Catheters were inserted into the right internal jugular vein, extending to the right atrium, and left carotid artery, extending into the aortic arch. Rats were given 1 week to recover from the surgery. Plasma glucose values were determined by using a glucose oxidase method (Beckman Glucose Analyzer II; Beckman Coulter). Plasma insulin concentrations were determined by a RIA Assay system (Linco). The rats were then fasted for 36 hrs, after which they were infused with 99% [6, 6-2H] glucose (1.1 mg/kg prime, 0.1 mg/kg) to assess the basal glucose and insulin turnover. The results, taken at the fed state (0 hr) and after fasting for 36 hrs, are presented in Table 8. The data demonstrates that both glucose and insulin were significantly reduced on treatment with ISIS 421062 in the fed state. In the fasted state, the glucose and insulin levels in both groups were equivalent.

TABLE 8
Basal glucose (mg/dL) and insulin (μU/mL) levels in the fed and
fasted states

| | Plasma glucose (mg/dL) | | Plasma insulin (μU/mL) | |
|---|---|---|---|---|
| | fed (0 hr) | fasted (36 hr) | fed (0 hr) | fasted (36 hr) |
| ISIS 421062 | 146 | 116 | 35 | 18 |
| ISIS 141923 | 160 | 116 | 57 | 19 |

### Effect on Insulin Sensitivity

Hyperinsulinemic-euglycemic clamp studies was conducted for 140 min with a primed/continuous infusion of insulin (400 mU/kg primed over 5 min and 4 mU/kg per min constant infusion) and a variable infusion of 20% dextrose spiked with 2.5%[6, 6-2H]glucose to maintain euglycemia. Once rats maintained euglycemia for 30 min, plasma samples were taken for clamp calculations. The hepatic glucose production was calculated by using the rate of infusion of [6, 6-2H]glucose over the atom percent excess in the plasma minus the rate of glucose being infused. The insulin-stimulated whole body glucose uptake was calculated by adding the total glucose infusion rate plus the hepatic glucose production. After the completion of the clamp, sodium pentobarbital was injected via the venous catheter administered at 150 mg/kg. After rats were completely anesthetized, tissues were extracted and frozen with the use of liquid cooled N₂ tongs. The samples were stored at ^{˜}80° C. until further analysis.

The results are presented in Table 9 and demonstrate that treatment with ISIS 421062 significantly increased insulin sensitivity, since the rate of glucose infusion (GINF) required to maintain euglycemia during the clamp was higher in the rat group treated with ISIS 421062 compared to that in the control group. This increase could not be accounted for by differences in endogenous glucose production or uptake in muscle or white adipose tissue (WAT). Furthermore, the results presented in Table 9 demonstrate effects of treatment with ISIS 421062 on other metabolic parameters without any increase in liver fat accumulation.

TABLE 9
Hyperinsulinemic-euglycemic clamp study

| | ISIS | ISIS |
|---|---|---|
| | 421062 | 141923 |
| Rat weight (g) | 359 | 356 |
| Fasting glucose (mg/dL) | 107 | 114 |
| Rate of glucose production (Ra) (mg/kg/min) | 7 | 7.3 |
| rate of glucose utilization (Rd) (mg/kg/min) | 29 | 23 |
| Glucose infusion (GINF) (mg/kg/min) | 26 | 19 |
| Basal insulin (IU/L) | 9 | 11 |
| Clamp insulin (IU/L) | 101 | 98 |
| Glucose uptake in soleus muscle (nmol/g/min) | 99 | 95 |
| Glucose uptake in WAT (nmol/g/min) | 1.8 | 2.3 |
| Clamp hepatic glycogen (mg/100 mg liver) | 45 | 52 |
| Clamp hepatic triglyceride (mg/g liver) | 3.5 | 3.7 |

### Effect on White Adipose Tissue Mass and Body Weight

Body weights of the rats in the two groups as well the weights of white adipose tissue were measured at the end of the study. The results are presented in Table 10 and demonstrate a decrease of 22% of WAT in rats treated with ISIS 421062 compared to the control group.

TABLE 10
Body weight and WAT weight

| | Body | | WAT (% |
|---|---|---|---|
| | weight (g) | WAT (g) | body weight) |
| ISIS 421062 | 344 | 2.7 | 78 |
| ISIS 141923 | 348 | 3.6 | 100 |

### Evaluation of Liver Function

To evaluate the impact of ISIS oligonucleotides on the hepatic function of the rats described above, plasma concentrations of transaminases were measured using an automated clinical chemistry analyzer (Olympus Clinical Analyzer). Measurements of alanine transaminase (ALT) and aspartate transaminase (AST) are expressed in IU/L. The results are presented in Table 11 and indicate that the oligonucleotides were well tolerated.

TABLE 11
Effect of antisense oligonucleotide treatment on transaminase
levels (IU/L)

| | ALT | AST |
|---|---|---|
| ISIS 421062 | 44 | 87 |
| ISIS 141923 | 40 | 94 |

Overall, the data demonstrates ISIS 421062 has beneficial effects of lowering glucose, insulin, triglycerides and fat mass with a concomitant increase in insulin sensitivity but without hypoglycemia following a prolonged fast. Therefore, PEPCK-M may be an attractive target for the treatment of diabetes and other similar metabolic disorders.

### Example 5

### Inhibition of PEPCK-M by siRNA in Primary Rat Hepatocytes

Inhibition of PEPCK-M mRNA by siRNA disclosed in Stark et al (J. Biol. Chem. 284: 26578-26590, 2009) was evaluated in vitro. Primary hepatocytes were isolated by standard procedures from Sprague-Dawley rats and cultured in 100 mm dishes. Cells were transfected using RNAifect (Qiagen; Valencia, Calif.) as per manufacturers recommendation with the following ratios: 3-6 ul siRNA (20 uM), 9 ul transfection reagent, 100 ul buffer EC-R, and 1 ml of Opti-MEM 1 with Glutamax (GIBCO Invitrogen Corporation, Carlsbad, Calif.).

Mitochondrial PEPCK (PEPCK-M) was targeted by two different siRNAs (Qiagen) with the following DNA templates: #1,5′-AACGTGAACAATTTGACATTA-3′ (SEQ ID NO: 131); #2,5′-TCCCATTGGGCTCGTACCAAA-3′(SEQ ID NO: 132). As a control, a non-silencing siRNA 5′-AATTCTCCGAACGTGTCACGT-3′ (SEQ ID NO: 133) (Qiagen) was used. Eight to 24 hours following transfection, the culture media was then changed back to RPMI 1640. After approximately 24 hours, RNA was isolated from the cells and PEPCK-M RNA transcript levels were measured. Quantitation of mRNA by reverse transcription and real-time PCR was performed using the following primers: PEPCK-M (5′-TTATGCACGATCCCTTTGCCATGC-3′(SEQ ID NO: 134), 5′-TCCTTCCTTTGGTACGAGCCCAAT-3′(SEQ ID NO: 135)), and GAPDH (5′-GTTACCAGGGCTGCCTTCTC-3′(SEQ ID NO: 136), 5′-GGGTTTCCCGTTGATGACC-3′(SEQ ID NO: 137)). PEPCK-M mRNA levels were reduced by 77% in cells treated with siRNA compared to the control.

### Effect on Gluconeogenesis

Hepatocytes were isolated and cultured in 6 well plates overnight. The next day, the medium was changed to DMEM without glucose. After a pre-incubation period, the medium was further changed to DMEM with different substrates (glutamine or alanine). Furthermore, glucose, glucagon, insulin, or glucagon+ insulin were individually added. The glucose levels at 0 hr and 3 hrs were measured. The rate of gluconeogenesis was calculated as glucose production (in mg) per mg protein supplied (in this case, glutamine or alanine) divided by the time (3 hrs). The data is presented in Tables 12 and 13. The data in each table demonstrates that there was a reduction in gluconeogenesis by approximately 60% on treatment with cells with siRNA (Glucose production in control vs siRNA-treated with no extraneous glucose supplied). This reduction occurred even in the presence of extraneous glucose, glucagon and/or insulin added to the medium.

TABLE 12
Rate of gluconeogenesis with glutamine as a substrate
(mg/mg of protein/hr)

| | | siRNA- |
|---|---|---|
| | Control | treated |
| Glucose (0 mmol) | 0.09 | 0.04 |
| Glucose (15 mmol) | 0.09 | 0.05 |
| Glucose (15 mmol) + glucagon | 0.13 | 0.10 |
| Glucose (15 mmol) + | 0.30 | 0.15 |
| glucagon + insulin | | |
| Glucose (15 mmol) + insulin | 0.07 | 0.05 |

TABLE 13
Rate of gluconeogenesis with alanine as a substrate
(mg/mg of protein/hr)

| | | siRNA- |
|---|---|---|
| | Control | treated |
| Glucose (0 mmol) | 0.12 | 0.05 |
| Glucose (15 mmol) | 0.19 | 0.09 |
| Glucose (15 mmol) + glucagon | 0.14 | 0.07 |
| Glucose (15 mmol) + | 0.10 | 0.14 |
| glucagon + insulin | | |
| Glucose (15 mmol) + insulin | 0.11 | 0.04 |

1.-40. (canceled)

41. A method of reducing phosphoenolpyruvate carboxykinase-mitochondrial (PEPCK-M) expression in an animal comprising administering to the animal a compound comprising an antisense oligonucleotide consisting of 10 to 30 linked nucleosides in length targeted to PEPCK-M, wherein expression of PEPCK-M is reduced in the animal.

42. A method of ameliorating a metabolic disease in an animal comprising administering to the animal a therapeutically effective amount of a compound comprising an antisense oligonucleotide consisting of 10 to 30 linked nucleosides in length targeted to PEPCK-M, wherein a metabolic disease is ameliorated in the animal.

43. The method of claim 42, wherein the metabolic disease is diabetes, obesity, metabolic syndrome, diabetic dyslipidemia, or hypertriglyceridemia.

44. The method of claim 42, wherein administering results in a reduction of insulin, insulin resistance, triglyceride levels, adipose tissue size or weight, body fat, glucose levels, insulin sensitivity, or any combination thereof.

45. The method of claim 44, wherein the reduction in body fat is a reduction in adipose tissue mass, adipocyte size or adipocyte accumulation or a combination thereof.

46. The method of claim 41, wherein the antisense compound has a nucleobase sequence at least 90% complementary to SEQ ID NO: 1, 2, or 3 as measured over the entirety of said antisense compound.

47. The method of claim 41, wherein the antisense oligonucleotide has a nucleobase sequence at least 95% complementary to SEQ ID NO: 1, 2, or 3 as measured over the entirety of said antisense compound.

48. The method of claim 41, wherein the antisense oligonucleotide consists of a single-stranded oligonucleotide.

49. The method of claim 41, wherein at least one internucleoside linkage of said antisense oligonucleotide is a modified internucleoside linkage.

50. The method of claim 49, wherein each internucleoside linkage is a phosphorothioate internucleoside linkage.

51. The method of claim 41, wherein at least one nucleoside of said antisense oligonucleotide comprises a modified sugar.

52. The method of claim 51, wherein at least one modified sugar is a bicyclic sugar.

53. The method of claim 51, wherein at least one modified sugar comprises a 2′-O-methoxyethyl or a 4′-(CH₂)ₙ—O-2′ bridge, wherein n is 1 or 2.

54. The method of claim 41, wherein at least one nucleoside of said antisense oligonucleotide comprises a modified nucleobase.

55. The method of claim 54, wherein the modified nucleobase is a 5-methylcytosine.

56. The method of claim 41, wherein the antisense oligonucleotide comprises:
a. a gap segment consisting of linked deoxynucleosides;
b. a 5′ wing segment consisting of linked nucleosides;
c. a 3′ wing segment consisting of linked nucleosides;
wherein the gap segment is positioned between the 5′ wing segment and the 3′ wing segment and wherein each nucleoside of each wing segment comprises a modified sugar.

57. The method of claim 41, wherein the antisense oligonucleotide is a first agent and further comprising administering a second agent.

58. The method of any of claim 57, wherein the second agent is lipid-lowering agent, anti-obesity agent or a glucose-lowering agent, or a combination thereof.

59. The method of claim 58, wherein the lipid-lowering agent is a HMG-CoA reductase inhibitor, cholesterol absorption inhibitor, MTP inhibitor, antisense compound targeted to ApoB, or any combination thereof; wherein the HMG-CoA reductase inhibitor is selected from atorvastatin, rosuvastatin, fluvastatin, lovastatin, pravastatin, or simvastatin; wherein the cholesterol absorption inhibitor is ezetimibe; wherein the anti-obesity agent is an appetite suppressant, Orlistat, Sibutramine, Rimonabant, or a combination thereof; wherein the appetite suppressant is diethylpropion tenuate, mazindol, orlistat, phendimetrazine, phentermine, sibutramine, or a combination thereof; and wherein the glucose-lowering agent is a therapeutic lifestyle change, PPAR agonist, a dipeptidyl peptidase (IV) inhibitor, a GLP-1 analog, insulin or an insulin analog, an insulin secretagogue, a SGLT2 inhibitor, a human amylin analog, a biguanide, an alpha-glucosidase inhibitor, metformin, sulfonylurea, rosiglitazone, a sulfonylurea selected from acetohexamide, chlorpropamide, tolbutamide, tolazamide, glimepiride, a glipizide, a glyburide, or gliclazide the biguanide metformin, a meglitinide selected from nateglinide or repaglinide, a thiazolidinedione selected from pioglitazone or rosiglitazone, or an alpha-glucosidase inhibitor selected from acarbose or miglitol.

60. A method for treating diabetes, obesity, metabolic syndrome, diabetic dyslipidemia, or hypertriglyceridemia in an animal comprising administering to said animal a therapeutically effective amount of an antisense oligonucleotide consisting of 10-30 linked nucleosides, and having a nucleobase sequence comprising at least 8 contiguous nucleobases of a nucleobase sequence selected from any one of SEQ ID NOs: 9-48, wherein administration of the antisense oligonucleotide treats diabetes, obesity, metabolic syndrome, diabetic dyslipidemia, or hypertriglyceridemia in the animal.
